SEQUENCE LISTING (18 sequences)

SEQ ID NO: 1 - DNA, 5471 bp, Nicotiana tabacum
  source 1..5471: mol_type = genomic DNA
atggccactactaaacagaaagtgacggcaccttctccttctccttcttcttcgactgcttcttgctgtccttccacttctatcctccgtcgtgaggcaacagcggccattgcagtcgtgggtgacggcctgcagaattggaccaacatcccctccgtcgacgagaagcagaaaaagacggcctcatcagctctagcgtcattgccaaccactgaacctctttccaccaatacctctaccaaaggtaattaataccatgctgctacataattattccatccgttctaatttatgtgtcttcatttgagtgggcacgaactttaacaaagcagagaatgacttttaaattttgtgctcttaaattaagaatatgtgtataatataccaaaatgtccttgaatcttgtggcttaaatatgtaatgtgaaaagttggaattaaaaagcaattaattaacatatagaaagagatattctttttaaacggactaaaaaggagaataatacacttaaattaaaatggaggaaataatatgtaccaaccgaaaggcctatatatatctatgcttgtaaagaatttttacattatccttataagtttaattacttgtagcaagtaatttgtctcatgaaactgtcttattttcatgtagttattgttgatgtaaaaattcttttacacattgaagttaaaattatgaactaatactagtttcttaaatttaactcttttccttcaggcttcagagaataattttctcggttcatgcatgttttttgtacgcatgtgtatatatgtagtatatagttatccttaaagaattcggtgcctctatcttttatttattatgtttatgataatcatagttgttacgaatgcatacaaaaaaaatattcagcttaattagaaccgcagctaaggcaatagtacctgctttaagggtccgtgagcgattaactaaatattataagatgaaggtggatgatttatttaatttaattagatcttgaatcttgagatcttgatatatgtgccgtttgaatgtagtgcttaattagtttcatgtttttctttaatttctgatcctttgagttgtggaatgattggttgcacaagaaacctatgagttgatgtgcatagcatttctaccgttttaaaactacggatctaggtaggatacctgaaataaataatttaagagtttaaagtgatcccaacttaaatttcgagaatcaaacttcttaatattgatcgtaaattcggacataaaattttgtatgttttcttttaaataaaatttacatatttgaatcttcatagtacaacaacaacaacaacccagtataatcccacttagtggggtctgagagggtagtgtgtacgcagaccttacccctaccctgggtagagagactgtttccaaatagaccctcggcatccttacctccaagaactttccaccttgctcttggggagactcgaactcacaacctcttggttgaaagtgaagaatgcttaccatcagagcaacccctcatagtaagtcacaataattaacaattgaaaatatttaaaaggcatatgaaaaaaacggtggtcagaaaaactcgttttactctcaaaatgcattaaataaattgggacagagagagtaagtaattacaagtaaattttttgacaaacattaattggtaatgtgataaaatggttaaaatttgcgtaaaacacttttaacattgtcattgcacacaacttaaataaattagtactttaatatgcttttgagctttccttaagtttaagataccctgaattgatgcgaatacatatagaagtgtatgtatgatttcctatataccactggtcctacacttaccattgcgaagtttgtttgaagattcatattaatgtttatatatggtgcaggtatccaaatcatgacaagggctcaaacctgccatcctttggaccctttatctgctgctgagatctcagtggctgtggcaactgttagagctgccggtgaaacacctgaggttcttattcccttttgtacatcacttgtgtgcggcaacaagaccactctgtcatttacaatatatatttattttaaaaaaatttcaggtcagagatgggatgcgatttattgaggtggttctggtagaaccagataaaagtgtagttgcattggcagatgcatatttcttcccaccttttcagtcatcattgatgccgagaaccaaaggaggatctcagattcctactaagcttcctccaaggagagctaggcttattgtttacaataagaaaacaaatgagacaagcatttggattgttgagctaaacgaagtacatgctgctgctcgaggtggacatcacaggggaaaagtcatcgcatccaatgttgtccctgatgttcagccacccatagtatgcacgcacttattactcacgtttagctgctgtgtgtttgttatgatccttatattaattttttttttaatttttattgtaggatgctcaagagtatgctgaatgtgaagctgtggtgaaaagttatcctccctttcgagacgcaatgaggagaaggggtattgatgacttggatcttgtgatggttgacccttggttcgttagaatcttcttcaccagtcttcccatttcttctctattgagaaatgccgttctgttttgacctgaatgcactttaatttcctacaggtgtgttggttatcatagtgaggctgatgctcctagccgcaggctcgcgaaaccacttgtattctgcaggacagagagtgactgcccaatggaaaatggatatgcaagaccagttgaaggaatatatgtgcttgttgatgtacaaaacatgaagattatagaatttgaagaccgaaaacttgtaccattacctccagttgacccactgaggaactacactgctggtgagacaagaggaggggttgatcgaagtgatgtgaaacccctacatattattcagcctgagggtccaagctttcgtatcagtggaaactacgtagagtggcagaaggtattctcattttctcagtcttgaattgctttctttgtctttatataacacattgacattgttcacttcctgcgtgacagtggaactttcggattggtttcacccctagagagggtttagttatacactctgtggcgtatcttgatggtagcagaggtcgtagaccaatagcacataggttgagttttgtagagatggttgtcccctatggagatccaaatgatccacattataggaagaatgcatttgatgcaggagaagatggccttggaaagaatgctcattcactgaagagggtcagttactatgctatgatctaaacattgcacataggtctgaatgctctttaactttaatcgctaacgtagaacaaaatcgttactgcagggatgtgattgtttagggtacataaagtactttgatgcccatttcacaaactttaccggaggagttgaaacgactgaaaattgtgtatgcttgcatgaagaagatcacggaatgctttggaagcatcaagattggagaactggccttgctgaagttagacggtctaggcgactaacagtgtcttttgtttgtacagtggccaattatgaatatgcattctactggcatttctaccaggtaagttaatgtgttttacatggaatatataaactaggtattagctctccctactagtgactggtgataatgaaatatagttacaagtttgcatttggaacgaagagtttgctgatcataactgtctttgattcagtcaccattttatatgcaattgcaggatggaaaaattgaagcggaagtcaaactcactggaattcttagtttgggagcattgcaacctggagaatatcgcaaatatggtaccacaattttaccaggtttgtatgcaccagttcatcaacacttctttgttgcacgaatgaatatggcagttgattgtaagccaggagaagcacacaatcaggtaactactatgactatttatagtaaacttcagttttcataatagtaactggtatgatgaatttttggaattacacaattcctttttttccaggttgttgaagtaaatgtcaaagttgaagaacctggcaaggaaaatgttcataataatgcattctatgctgaagaaacattgcttaggtctgaattgcaagcaatgcgtgattgtgatccattctctgctcgtcattggattgtaaggatccacacccgagtaactttatttattttatctcactatgaccagtgcatgctacataaatttgactatgaactatctgaatttgtaggttaggaacacaagaacagtaaatagaacaggacagctaacagggtacaagctggtacctggtccaaactgtttgccactggctggtcctgaggcgaaatttttgagaagagctgcatttctgaagcacaatctatgggttacacaatatgcacctggagaagattttccaggaggagagttccctaatcaaaatccccgtgttggcgagggattagcttcttgggtcaagcaagaccggcctctggaagaaagtgatattgttctctggttagttctctagacaacaatcaaaactatcttactgcaatggtagtgtttgggatattgaaaaaagttctgaccagcaccgtttattgtaggtatatttttggaatcacacatgttcctcggttggaagactggcctgttatgccagtagaacacattggttttgtgctacaggtacttggcaatgctatgttagcttttactgtttatttcccatcttaaacatctcagtgatttagttgattagaactcaatactggaatagcacaaaagattaaaggagaaagaattatgcaagatattcctgtaagagctcttaaaagaaaaagcaatttgaaagaatgctcattcactgaatcaacaaatagtaaatattgcatgaaatgatcttagaattacatcacttactttagccggtctatcgaaaacagcttctctgccttcccaagatagaggcgtatacactaacctctccaaaccccacttgtgggaattcacatgctttttttgttgttgttgttgttgttgttgttgttgttgttgttgttgttgtactacttgaattgcatcacttgtgccaacctgcataagttgatggtcccaatctagggaagaccttatccaactcaaatacaatctatgcagtgatactctcattctgaatttgttcttgtctttccagtttggttaatgttttttctgtttgttgttgcagccacatggatactttaactgctctccggctgttgatgtccctccgccctttgcatgcgactcagaaagcagagacagtgatgttactgaaactagtgtagcaaagtccactgccactagcttgctggccaagctttga

SEQ ID NO: 2 - DNA, 2373 bp, Nicotiana tabacum
  source 1..2373: mol_type = other DNA
atggccactactaaacagaaagtgacggcaccttctccttctccttcttcttcgactgcttcttgctgtccttccacttctatcctccgtcgtgaggcaacagcggccattgcagtcgtgggtgacggcctgcagaattggaccaacatcccctccgtcgacgagaagcagaaaaagacggcctcatcagctctagcgtcattgccaaccactgaacctctttccaccaatacctctaccaaaggtatccaaatcatgacaagggctcaaacctgccatcctttggaccctttatctgctgctgagatctcagtggctgtggcaactgttagagctgccggtgaaacacctgaggtcagagatgggatgcgatttattgaggtggttctggtagaaccagataaaagtgtagttgcattggcagatgcatatttcttcccaccttttcagtcatcattgatgccgagaaccaaaggaggatctcagattcctactaagcttcctccaaggagagctaggcttattgtttacaataagaaaacaaatgagacaagcatttggattgttgagctaaacgaagtacatgctgctgctcgaggtggacatcacaggggaaaagtcatcgcatccaatgttgtccctgatgttcagccacccatagatgctcaagagtatgctgaatgtgaagctgtggtgaaaagttatcctccctttcgagacgcaatgaggagaaggggtattgatgacttggatcttgtgatggttgacccttggtgtgttggttatcatagtgaggctgatgctcctagccgcaggctcgcgaaaccacttgtattctgcaggacagagagtgactgcccaatggaaaatggatatgcaagaccagttgaaggaatatatgtgcttgttgatgtacaaaacatgaagattatagaatttgaagaccgaaaacttgtaccattacctccagttgacccactgaggaactacactgctggtgagacaagaggaggggttgatcgaagtgatgtgaaacccctacatattattcagcctgagggtccaagctttcgtatcagtggaaactacgtagagtggcagaagtggaactttcggattggtttcacccctagagagggtttagttatacactctgtggcgtatcttgatggtagcagaggtcgtagaccaatagcacataggttgagttttgtagagatggttgtcccctatggagatccaaatgatccacattataggaagaatgcatttgatgcaggagaagatggccttggaaagaatgctcattcactgaagaggggatgtgattgtttagggtacataaagtactttgatgcccatttcacaaactttaccggaggagttgaaacgactgaaaattgtgtatgcttgcatgaagaagatcacggaatgctttggaagcatcaagattggagaactggccttgctgaagttagacggtctaggcgactaacagtgtcttttgtttgtacagtggccaattatgaatatgcattctactggcatttctaccaggatggaaaaattgaagcggaagtcaaactcactggaattcttagtttgggagcattgcaacctggagaatatcgcaaatatggtaccacaattttaccaggtttgtatgcaccagttcatcaacacttctttgttgcacgaatgaatatggcagttgattgtaagccaggagaagcacacaatcaggttgttgaagtaaatgtcaaagttgaagaacctggcaaggaaaatgttcataataatgcattctatgctgaagaaacattgcttaggtctgaattgcaagcaatgcgtgattgtgatccattctctgctcgtcattggattgttaggaacacaagaacagtaaatagaacaggacagctaacagggtacaagctggtacctggtccaaactgtttgccactggctggtcctgaggcgaaatttttgagaagagctgcatttctgaagcacaatctatgggttacacaatatgcacctggagaagattttccaggaggagagttccctaatcaaaatccccgtgttggcgagggattagcttcttgggtcaagcaagaccggcctctggaagaaagtgatattgttctctggtatatttttggaatcacacatgttcctcggttggaagactggcctgttatgccagtagaacacattggttttgtgctacagccacatggatactttaactgctctccggctgttgatgtccctccgccctttgcatgcgactcagaaagcagagacagtgatgttactgaaactagtgtagcaaagtccactgccactagcttgctggccaagctttga

SEQ ID NO: 3 - protein, 790 aa, Nicotiana tabacum
MATTKQKVTAPSPSPSSSTASCCPSTSILRREATAAIAVVGDGLQNWTNIPSVDEKQKKTASSALASLPTTEPLSTNTSTKGIQIMTRAQTCHPLDPLSAAEISVAVATVRAAGETPEVRDGMRFIEVVLVEPDKSVVALADAYFFPPFQSSLMPRTKGGSQIPTKLPPRRARLIVYNKKTNETSIWIVELNEVHAAARGGHHRGKVIASNVVPDVQPPIDAQEYAECEAVVKSYPPFRDAMRRRGIDDLDLVMVDPWCVGYHSEADAPSRRLAKPLVFCRTESDCPMENGYARPVEGIYVLVDVQNMKIIEFEDRKLVPLPPVDPLRNYTAGETRGGVDRSDVKPLHIIQPEGPSFRISGNYVEWQKWNFRIGFTPREGLVIHSVAYLDGSRGRRPIAHRLSFVEMVVPYGDPNDPHYRKNAFDAGEDGLGKNAHSLKRGCDCLGYIKYFDAHFTNFTGGVETTENCVCLHEEDHGMLWKHQDWRTGLAEVRRSRRLTVSFVCTVANYEYAFYWHFYQDGKIEAEVKLTGILSLGALQPGEYRKYGTTILPGLYAPVHQHFFVARMNMAVDCKPGEAHNQVVEVNVKVEEPGKENVHNNAFYAEETLLRSELQAMRDCDPFSARHWIVRNTRTVNRTGQLTGYKLVPGPNCLPLAGPEAKFLRRAAFLKHNLWVTQYAPGEDFPGGEFPNQNPRVGEGLASWVKQDRPLEESDIVLWYIFGITHVPRLEDWPVMPVEHIGFVLQPHGYFNCSPAVDVPPPFACDSESRDSDVTETSVAKSTATSLLAKL

SEQ ID NO: 4 - DNA, 6541 bp, Nicotiana tabacum
  source 1..6541: mol_type = genomic DNA
atggccactactaaacagaaagtgacggcaccttcttcttcgactgctccttgctgtccttccacttccatcctccgtcgtgaggcgacagctgccgttgcaggcgtgggcgacggcctgcaaaattggaacaacgtcccgtccgtggatgataagcagaaaaagacggcctcatcagctctagcgtcattggcaagcactgaacctctttcctccaatacctctaccaaaggtaataccatactgcaacatatatctctcgctaattattccaacagtttcaatttatgtgtcttaacttgagtaggcatgaagtttaattaacaaagtcgagaatgacttttaaattaaatcttgtggtattaaactaaagatgtgcgtaatgtaccaaaatgcccggtgaaattcaaaaatagccaaatttatagtaccaaaatgcccggtgaaattcaaaaatagccaaatttatagtggtaattaaaaaatagctacagttttaaaaataatcaaaatttagccatttttcatgtaaaaataaatttgaacgaaaacactgttcaaaatccgaaaaatattccagcataatatactggagttccagtataatatactggtccagcataatatgctggaagttcatacacatgtgctccaatctcccgtatattatgttgaaactttttgtgtgttggagtttcagcataatatgctgaagttcatacacaggtgtaccaatttccagtatattatgctggaactttccgtgttacaaagttccagcaaaatattggctatttttcaatgactttataaacgctgactatttttcaattatcagtgcgaaaactggctagtccgtgatattttacaaaatgctcttgaattttgtggattaaatatgtcatgtgaaaagttggagttagaaagttattaacatagaaagagatttttttttttttttttgaaattgcctaaaaaggagaataatatacataaattgaaacggacgaaaggcgtatatatatatatatatatatatgcttgtaaagaatttttacattattcttataattttaattacttgtagcaagtaatttgtctcatgaagctgtcattttttcatacggttacctttcatgtaaaaattcttttatactgtaaatgtttagaagttaaaattataaactaataggggtactagtttttaaaatttatctcttttccttcaggcttcagagaataatatttctcggtgcatgtcttgtacgcatgtgtatatatgtagtatagttatccttaaagaattcggtgcctctatctttttatttattatattatgataatcatagttgttacgaatacatacaaacaaaatcttcagtttagaaccacagctaaggcaatagtacctgctttaagggtccgtgagcgattaactaaatataagatgaaggtggatgatttatttaatttaattagatcttgaatcttgtgttcttgatatatgtgccgtttgaatgtagtgcttaattagtttcatgtttttctttaatttctgatcctttgagttgtggagtgattggttgcccaagaaacctatgagttgttgttcgtagcatttctaccgttttaaaactaaggatatgtcacttaataaattgtttaagagtttaacggtgtagaaaaggtttactaaggtcacttaatactccctccatcgtgatttatatgatctagtttgaattttaagagtcaaatttcttaattttgatagtaaattcgaacataaaatctttaagttttttgaaataaaatttacgtatttggaaacttcttaaaaagaactataagttacaataattaacaattcaaaatatttaaaaggcatatgaaaaaattacggtcaaagaaaaactcgttttgactctcgaagttcgaattggatcgtataagtaattataggtatatgataattaaaaatggttaaaattcgtgaaaacacttttaacattgtcattgtacataacttatagtacttaaacattctcttgaactttcctttagataccctaaatagatgtgaatacataagtgcatgtatgatttcctatatatagctggttctacacttatgatttcctatatatagctggttctacacttaccattgccattgcttgaagattcatagagtaatattaatggtgcaggtatacaaatcatgacaagggctcagacttgccatcctttggaccctttatctgctgctgagatctctgtggctgtggcaactgttagagctgccggtgaaacacctgaggttctgcttcttttgtacatcacttgtgtgtagaacttctttttttccgtatcggaccaacaaaggactaaatgtcactagaacgtggcgacaaaaccactctgtctgtcacttacaacatatttgtggaactagttttatttgaacaaagtgacatttttttccactaattaactcttgttttatttttcctttcaaaaaaaaaaataataataatttcaggttagagatgggatgcgatttattgaggtggttctgttagaaccagataaaagtgttgttgcattggcagatgcatatttcttcccaccatttcagtcatcattgatgcccagaaccaaaggaggatctctaattcctactaagcttcctccaaggagagctaggcttattgtttacaataagaaaacaaatgagacaagcatatggattgttgagctaaatgaagtacatgctgctgctcgaggtggacatcacaggggaaaagtcatctcatccaatgttgtccctgatgttcagccacccatagtatgcacttaactcagctttctttctctagaacatcagataatttccggcatcgtgaaatgttaggctctatagctgctgtgtgtttgttatgatgcttatattaattttgttttttctgattgtaggatgctcaagagtatgctgaatgtgaagctgtggtgaaaagttatcctccctttcgagacgcaatgaggagaaggggtattgatgacttggatcttgtgatggttgacccttggttcgttagaatcttcttcaccaatcttcccatttcttctctattgagaaatgccactctgttttgacctgaatgcacttttatttcctacaggtgtgttggttatcatagtgaggctgatgctcctagccgcacgctcgcgaaaccacttgtattctccaggacagagagtgactgcccaatggaaaatggatatgcaagaccagttgaaggaatatatgtgcttgttgatgtacaaaacatgcagattatagaatttgaagaccgaaaacttgtaccgttacctccagctgatccactgaggaactacactgctggtgagacaagaggaggggttgatcgaagtgatgtgaaacccctacatattattcagcccgagggtccaagctttcgtatcagtggaaactacatagagtggcagaaggtattctcattttttcagtcttgttttggtatgaagcattctttgtctttatataatacattgacattgttcacttcctgcgtgacagtggaactttcggattggtttcacccctagagagggtttagttatacactctgtggcgtatcttgatggtagcagaggtcgcagaccaatagcacataggttgagttttgtagagatggttgtcccttatggggatccaaatgatccacattataggaagaatgcatttgatgcaggagaagatggccttggaaagaatgctcattcactgaagagggtcagttactattctatgttctaaacattgcagatctgaattcttttcactttaattgctaatgtcgaacaaaataattgctgcagggatgtgattgtttagggtacataaagtactttgatgcccatttcacaaactttacgggaggagttgaaacgactgaaaattgtgtatgcttgcatgaagaagatcacggaatgctttggaagcatcaagattggagaactggccttgctgaagttagacggtctaggcgactaacggtgtcttttgtttgtacagtggccaattatgaatatgcattctactggcacttctaccaagtaagttaatgtgttttacatggattatataaactaggtattagctctcccaacacgactggtgataatgaaacataattacaagtttgcatttggaatttcttcagggaaacgaagagtttgctgatcataattgtctttgattcagttaccattttacaactgtttgtacgctatatgcaattgcaggatggaaaaattgaagcggaagtcaaactcactggaatacttagtttgggagcattgcaacctggagaatatcgcaaatatggtaccacaattttaccagggttgtatgcaccagttcatcaacacttctttgttgcgcgaatgaatatggcagttgattgtaagccaggagaagcacacaatcaggtaactactatgactatttatagtaaacttcagttttcataatagtaactggtatagatcaggttccttctaatctggtatgattaattttttgcgttatacaaatatttttttccaggttgttgaagtaaatgtcaaagttgaagaacctggcaaggaaaatgttcacaataatgcattctatgctgaagaaacattgcttaggtctgaattgcaagcaatgcgtgactgtgatccattctctgctcgtcattggattgtaagaatccacacctgactgtatttacctttaattgcttttatctcactatgataactagaaaggaggatagacctcttgtttctctgtaaagcattttatataacaagtttttttactgaactgtatgttctggaactgaaactgggcacctttcttatgtcgtccaatttttgactaatttgatcctaaagacccttttgatgcttggttgtggttttgatcttaagaccagtgcatgctacataaatttgacaatgaactatctgactttgtaggttaggaacacaagaactgtaaatagaacaggacagctaacagggtacaagctggtacctggtccaaactgtttgccattggctggtcctgaggcgaaatttttgagaagagctgcatttctgaagcacaatctatgggttacacaatatgcacctggagaagaatttccaggaggagagttccctaatcaaaatccccgtgttggcgagggattagcttcatgggtcaagcaagaccggcctctagaagaaagtgatattgttctctggttagttctctagacaacaatcaaaactatcttactgcagtggatatttgagatttaaaatagtctgcaaaatgtcaaatggtcgaaactttctagacgtggaaccttctaaagtacttgaacaaatttatatcaaattcacgcatgactagttaaatattctgtaaacttttctttttctttctttggtggatattgaaaagtatgctttgaagttttgacaacaccgtttattgtaggtatatttttggaatcacacatgttcctcggttggaagactggcctgttatgccagtggaacacattggttttgtgctacaggtagttgagaatgctatgttagcttttactgtttatttcccatcttaaacatctcagtgatttagctgagtagaactcaatactggaaaagcatattagattaaaggtaaagaattatgcaagatattccggtacgagatcttaaaagaaaaagcaattcgaaagaatgctcattcactgaagaggctctggggaagggattcttaatcaacaaatagtaagtattgcatgaaatgatcttagaattctccttatatatcatactctctaaaccaggttatgcgtagcctcttagtgataaatacctccatttattcccttcacttgaattacatctcccactttatccgagggtctattggaaacaatctctatgcctttccaagacaggggtaaggcctgcgtacacactaccttctccagaccccacttgtgggaattcactggatttttgttgttgttgttgttgttgtacttgaattacatcacttgcgccaacctgcataagttgatggtcccaatctagggaagacaatccaactcaaatacaatctatgcagtgatactttaaatctgaaatttacatccttaacaagataagatgtaccacttagtaagaccaacttcagcatcagtagttttgctcgaaataatgaacatggtatattatactaaatgcagcttgtgaatttgttcttgtctttccagtttgggtaatgttttttttttttttttttttttttttttttctgttggtgcagccacatggattctttaactgctctccggctgttgatgtccctccgccctctgcatgcgactcggaaagcagagacagtgatgttactgaaaccagtgtagcaaagtccactgccactagcttgctggccaagctttga

SEQ ID NO: 5 - DNA, 2361 bp, Nicotiana tabacum
  source 1..2361: mol_type = other DNA
atggccactactaaacagaaagtgacggcaccttcttcttcgactgctccttgctgtccttccacttccatcctccgtcgtgaggcgacagctgccgttgcaggcgtgggcgacggcctgcaaaattggaacaacgtcccgtccgtggatgataagcagaaaaagacggcctcatcagctctagcgtcattggcaagcactgaacctctttcctccaatacctctaccaaaggtatacaaatcatgacaagggctcagacttgccatcctttggaccctttatctgctgctgagatctctgtggctgtggcaactgttagagctgccggtgaaacacctgaggttagagatgggatgcgatttattgaggtggttctgttagaaccagataaaagtgttgttgcattggcagatgcatatttcttcccaccatttcagtcatcattgatgcccagaaccaaaggaggatctctaattcctactaagcttcctccaaggagagctaggcttattgtttacaataagaaaacaaatgagacaagcatatggattgttgagctaaatgaagtacatgctgctgctcgaggtggacatcacaggggaaaagtcatctcatccaatgttgtccctgatgttcagccacccatagatgctcaagagtatgctgaatgtgaagctgtggtgaaaagttatcctccctttcgagacgcaatgaggagaaggggtattgatgacttggatcttgtgatggttgacccttggtgtgttggttatcatagtgaggctgatgctcctagccgcacgctcgcgaaaccacttgtattctccaggacagagagtgactgcccaatggaaaatggatatgcaagaccagttgaaggaatatatgtgcttgttgatgtacaaaacatgcagattatagaatttgaagaccgaaaacttgtaccgttacctccagctgatccactgaggaactacactgctggtgagacaagaggaggggttgatcgaagtgatgtgaaacccctacatattattcagcccgagggtccaagctttcgtatcagtggaaactacatagagtggcagaagtggaactttcggattggtttcacccctagagagggtttagttatacactctgtggcgtatcttgatggtagcagaggtcgcagaccaatagcacataggttgagttttgtagagatggttgtcccttatggggatccaaatgatccacattataggaagaatgcatttgatgcaggagaagatggccttggaaagaatgctcattcactgaagaggggatgtgattgtttagggtacataaagtactttgatgcccatttcacaaactttacgggaggagttgaaacgactgaaaattgtgtatgcttgcatgaagaagatcacggaatgctttggaagcatcaagattggagaactggccttgctgaagttagacggtctaggcgactaacggtgtcttttgtttgtacagtggccaattatgaatatgcattctactggcacttctaccaagatggaaaaattgaagcggaagtcaaactcactggaatacttagtttgggagcattgcaacctggagaatatcgcaaatatggtaccacaattttaccagggttgtatgcaccagttcatcaacacttctttgttgcgcgaatgaatatggcagttgattgtaagccaggagaagcacacaatcaggttgttgaagtaaatgtcaaagttgaagaacctggcaaggaaaatgttcacaataatgcattctatgctgaagaaacattgcttaggtctgaattgcaagcaatgcgtgactgtgatccattctctgctcgtcattggattgttaggaacacaagaactgtaaatagaacaggacagctaacagggtacaagctggtacctggtccaaactgtttgccattggctggtcctgaggcgaaatttttgagaagagctgcatttctgaagcacaatctatgggttacacaatatgcacctggagaagaatttccaggaggagagttccctaatcaaaatccccgtgttggcgagggattagcttcatgggtcaagcaagaccggcctctagaagaaagtgatattgttctctggtatatttttggaatcacacatgttcctcggttggaagactggcctgttatgccagtggaacacattggttttgtgctacagccacatggattctttaactgctctccggctgttgatgtccctccgccctctgcatgcgactcggaaagcagagacagtgatgttactgaaaccagtgtagcaaagtccactgccactagcttgctggccaagctttga

SEQ ID NO: 6 - protein, 786 aa, Nicotiana tabacum
MATTKQKVTAPSSSTAPCCPSTSILRREATAAVAGVGDGLQNWNNVPSVDDKQKKTASSALASLASTEPLSSNTSTKGIQIMTRAQTCHPLDPLSAAEISVAVATVRAAGETPEVRDGMRFIEVVLLEPDKSVVALADAYFFPPFQSSLMPRTKGGSLIPTKLPPRRARLIVYNKKTNETSIWIVELNEVHAAARGGHHRGKVISSNVVPDVQPPIDAQEYAECEAVVKSYPPFRDAMRRRGIDDLDLVMVDPWCVGYHSEADAPSRTLAKPLVFSRTESDCPMENGYARPVEGIYVLVDVQNMQIIEFEDRKLVPLPPADPLRNYTAGETRGGVDRSDVKPLHIIQPEGPSFRISGNYIEWQKWNFRIGFTPREGLVIHSVAYLDGSRGRRPIAHRLSFVEMVVPYGDPNDPHYRKNAFDAGEDGLGKNAHSLKRGCDCLGYIKYFDAHFTNFTGGVETTENCVCLHEEDHGMLWKHQDWRTGLAEVRRSRRLTVSFVCTVANYEYAFYWHFYQDGKIEAEVKLTGILSLGALQPGEYRKYGTTILPGLYAPVHQHFFVARMNMAVDCKPGEAHNQVVEVNVKVEEPGKENVHNNAFYAEETLLRSELQAMRDCDPFSARHWIVRNTRTVNRTGQLTGYKLVPGPNCLPLAGPEAKFLRRAAFLKHNLWVTQYAPGEEFPGGEFPNQNPRVGEGLASWVKQDRPLEESDIVLWYIFGITHVPRLEDWPVMPVEHIGFVLQPHGFFNCSPAVDVPPPSACDSESRDSDVTETSVAKSTATSLLAKL

SEQ ID NO: 7 - DNA, 7565 bp, Nicotiana tabacum
  source 1..7565: mol_type = genomic DNA
atggccgcaactttgcacaaggtgactcctcctcctccgactactactgcttcttgctgcccttcggcttccgcttctgtcatccgtcgtgagtccgccgcagcctccgtcgtggaggacgatcagcagaaacaaacgccggcgctgacgtcattggttaactctcaacctccttcctccaatccctccggcaaaggtataaatgcctttcgactatataaaattaagtttctaccctctattcaaacaatgtgtgtgtgtgtgtgtgttttagccggccgtcggacttcggagtatatgtatgtgtgcaattgcacaagttgttgtctctttctgtaatttgggcatctgcatgtttgattttcgtgtgcttaccgaatataaatgacagcttcgatgattttgttcatctgcactgacaaattatttctatatcctcgtaatttatatacctgtagtcttaattttggtatacatggagaaggaaattctattgaataagctacagtagaattacggtccttaaacatttgtttagtctcaacaatcagggtttacttttggatgttgccagtgtagttgcacaattcctgcacttatatttttagatcaaataatatattatttagatcactacaatatattttacccctgcaattattaacgtagagtcttgtccttaatttgaattgacgatcaatcaaatagttaactaattcctaatttcaaacttggtgatatcaaatcatttgtatctttgtatatccatttcactctatactcgtgtatttttctccagaggatttatactgtaatagttaaaactatttttaggttgtatgtcaacctaccatgtccttccttgttttgtgaaacttaagtaaatgaaattccttttgaaaagaataagaaaagattttttttcctttaaaaattaagaaaagataaaaacaaagtgatacattaatggagggagcttgttattggagtagttggtaagatgttcagtgcttgacgattccatgtttatgcagggaaacaaatcatgccaagagctcatacatgccatcctttggaccctttatctgctgctgaaatctctgtggctgtggcgaccgtcagagctgccggtgaaacacccgaggttcttaatcactgtctttttgtcatccatttttgcgttgaacaattgaacaagtattagttgaacaacaggacatcatttgctctcaattcccctaattgttttcatttatttattttttgcaaaaacattttaggtcagagatggcatgcgctttattgaggtggttctactggaacctgataaaagtgtcattgcactggctgatgcctatttcttcccacctttccaatcttcgttgatgtccagaaggaaaggagggcttcccattcctactaagcttcctccaaggcgagctagacttattgtatataataagaaaacaaatgagacaagcatatggattgttgagctagctgaagtacatgctgctgctcgaggtggacatcacaagggaaaagtgatttcatcctatgttgttccagatgttcagccacctatagtaagcaatttctcgtctttagaactttaagagtttagaagtacatgaacaacagatgcaattgggttatcaaagaactgaactgagaacaccaagtaagagcaacccaaagttcttattacgttttaaaccaaatgctttcacacttcactctaataattgctcatgaataatgaattttaactatattttctgaaacattaccgatttcatttattggtatactttagaacgttcacttggctagatcaaccgattaattaagttatgaacatttttgaagttgtgaatacctccgtcctaatggaacatttaattaattttcctagcagtaagttggttgttcttttgtttctatcaactggtcttgtacaactggtgctctagagattctaaacactctatgaaagaaaagagtcggaagaatttgcatttttcttgtacgagcatacggtcttggggcaacaaggagaaaggacacaagccaaattggacatccattctgattcgacaagccgattaagtgtttttcgtgctatatatacttttgcttacttatattgctctcttcttgatgaactgtgttttctttcccatcatatggaaagtagagcatacttatacaccatagtcagaagtaatttttgcatatatagtctgtctcaactctcaagtcaacccaaaaaatgttaaagtttgcaacttttaagttttttggcttaagtgagttgtggtataggtcagctctttaaatttcaataattctcaaattttacatatcatcttaactttgtactgattgtaggatgcacaagagtatgcagactgtgaagctgtagttaaaaattatcctccttttagggaagcaatgaagagaaggggtattgatgacatggatcttgtgatggtggacccctggttagttaaatacccttgatttttctttcttctcctttaagaaccgatttcttatttcattgcatttcgacctgaaaatgctttccgcacaggtgcgttggttttcacagtgaggctgatgctcctagccgcaggcttgccaaaccgctagtattctgcagatcagagagtgactgcccaatggaaaatggatatgcaagaccggttgaaggaatttatgtacttgttgatgtgcaaaaaatgcaggtgatagagtttgaagaccgcaaacttgtacctttacctccagctgatccactgaggaattacactgctggtgagacaagaggaggggtcgatcgaagtgatgtgaaacccctccagattattcagccagagggtccaagctttcgagtcaatgggaactatgtggagtggcaaaaggtaaattcttttttccagtctgatatgcaatgaaggattatttgcttatacatctaacatgtcattctttcactttgttttgacagtggaacttccgagtaggtttcacccctagggagggtttggttatacactctgtggcatatattgacggtagtaggggtcggagacccatagcccatagattgagttttgtggagatggttgtcccctatggggatccaaatgacccacattacagaaagaacgcttttgatgcaggagaagatgggctcggaaagaatgctcattcacttaagagggtaactgacatttctatggttcaattatggatgacttttgggaatgtaagtgctaatctaacaaaatcacccttgcagggatgcgattgtttaggatacataaagtactttgatgccaattttgcaaattttaccggaggagtagaaaccactgaaaattgtgtatgtttgcatgaagaagatcacgggatgctctggaagcatcaagattggagaactggccttgcagaagttagacggtctagacgacttacagtttcttttatttgcactgtggccaattatgaatatggattctactggcacttttaccaggcaagaataagaaaatcattatcaggaaatcctgtgtttgcatgttctaaaaaaattaggtaacaatgaagttttattacaagttcacattttatttagaacacttgagggaaaagaaagcaagcagaagcaagataaagaatttcttgtaacttcggtcgtgtttatgaataatgttttctgagagacaaactcattcctggggagaaaaaaccctcattcttggccaaatgcttgaaacaaaaccaatgttatcattctgttataattgagttgataccaataatagctctactacttgcttatgccttttttttgtgggcgaggggggatttgctaagccaaagacaagtgaatatcagaagtttacatctatccccgagaaaaaaattcagaatttatgctcatttattttgtataaatgtctttactgattgtaagtacgtttagctccacttaccattttgaaatgatctgtaaattttgtggttgcaggatgggaaaattgaagcagaaatcaaactcacaggaattctcagtttgggagcactgcctcccggagagtctcgtaaatatggcaccacaatagcaccgggattgtatgcacctgttcatcaacacttctttgttgctcgtatgaatatggcagttgattgtaaaccaggagaagcacacaatcaggtatttagtggaacaagttaccatgtccacccggaagttaaagtaatttcataccttcagtttcccagttcttaactttctaatctgagttgttagtcctttgtctaggttgttgaagttaatgttagagttgaagaacctgggaaagaaaatgttcacaacaatgcattctatgctaaggaaacagtgcttacgtctgaattgcaagcaatgcgtgactgtgatactttatctgctcgtcattggattgtaagaagccttacttgactgtatcctcctttctttttatagaactgttcttcaggttacttcctggcacagtatctgctgagatatgtactcatagtagactgtggtccttaaacatggccactgatttttgagggagctaaatgcaatgtgtagaacctccaccttaatgctagtcttgtcatatgagaattgtttctccaaaaaaatgattttaagaactttctgtgacttgtatggttttggcataagagtatattatgtttcattcttctgaaaatcacgaacttttgttgagttttgctctctttggctagagatgtgtttggtaacgaaaacaacaaacccagtttagtcccacaagtggggtttagggagggtagagtgtatgcagatcttgccccctaccttgtgaaggtagagaagttgttcccgatggacccttcgctcaaggaaagatagaaaaagaaacaatagcatcaagcagtaacaacaacacgatgatagattaaccgaagcgaaagaaacaagaatagggataagaataagaaatagaaacaacaggtagtaaaagaaaactaagcataaggaaatacaagagcaatactaatactactgctatggaaaagaaatacgctcaactacctgctaaccttccaccgtaagattgttgatgaaaatttcaattgcaaattcatttagatagggcggttgaactttattgtttcagcatgtggtattgtggagtcaatttttagattccaaaatctgctaactcaaaaaccaaggagaagtggaagaaattgaatactcgcatccccaacctagtaacagagccatcgatgtgactgcgaacatctgattttgcaggttaggaacacaagaacatccaatagaacaggacagctaacagggtacaagctggtacctggccctaactgtttgccattggctggtcctaaggctaagtttttgagaagagctgcatttttgaagcacaatctatgggttacaaaatatgcacccggagaagattttcccgggggagagttccctaatcaaaatccacgtgttggtgagggattagcttcttgggttaagcaagatcgttctctggaagaaagtgatgttgttctctggttcgtttccgacaactgtcaaattatgttataacattggagttgcaggtttcagattgttttacctcacacacaaacttaactttcataaagagctagatgtaaaacttctaattccttggaaacccttatatacatgtttgagaagttccctattccttgaaattctgatctacacagattattgcaggtatgtttttggaatcacacatgttcctcggttggaggactggcctgttatgccagttgaacatatcggttttatgcttcaggtattctggaatatggatgttttgtgtttattttgtatgtactggtgtttttctccttatctttgtcatcatctaacttctcgttatctattgtgttaccatttacttggtactaaagagctgattatgtattgtatcaattttttgcaataactcaactcattcatacattcgctatgtgtatgttacaataactagccaaacataaacatagagtgtagcattggaagtattggcagtggagtagaaaggatcgtgaatatccacagagaacttgtaaaatcaaagcaattgggagctggtagtagaaatgagaaggttctttttcaagttgttatcttgacattaacctattcaatagtccgttggatttgcttgttgctttacttttactgttccttaagctgagggtctattggaaacaacctctctgcctttatagggtaggggtaaggctgcgtacacactaccctccccagactgcacgtatgggattaagctgggttttttgttgttgtaatagtctgttggatgttttactctgttgaacaaggaagagggtttttggggagagggagagtacggaagttgttaataaactgcaatggctggaaaatgtacctcagcattttttttttgtatcagtcctaataggccaagaccttaagtctggatcaggttaaccacagtaaatttagtgataaaactctccatcctttttaattactgaaatcttgtaatcttttttcgaaattgctggctaaactagagaattatatcccttcctcagaacctgtttaagtttattgcccaagtttagcaatgactttatccaactccaaaaacctgctgtataatattcattgtgaaaactgttaagattcatggaaaacttaactccacgtatgtaaaattttgcatagccggtcttcaatccggataaagaagagggttgtgatatcttgatggctaaaccaataagttccctaactaatttggtattaacatagttgattcattgatcgatataacatggaaattcggttgaaaagggattagttctatactcataagtagagttggcaaaatggttaaaagaaaacagttatccacccatattatccatcaaaaaatgagttgaataatgaactttttaaaaacgggtcaaatatggataagacccatattatccaattagaaaatgaataaccaacgggtttaacttttacatttataaagcctcaaattggaggttcctcaagttagaaagactaggaattctctcaaaagtaatcatattcaagaagccatggataatatggatatttatattattcgccggttaaccagtgttttatccgtattaaatatgtgtcgggtcggataatttatctattttttgcattacccgttttcagctcgcccatatccgacccgacctgcctgccacccctactcataagtcataatagaaggtggatggttctgtttgtccataacctgttcagtttaaagaatgattatttctgcttgttggtgcagccacatggattctttaactgctctcccgctgtagatgtacctcctcctcggggatgtgacatggaaatcaaagacagtgatggttcagaaaatggtgtagcaaagcccactcccagtaatttgatggccaagctttga

SEQ ID NO: 8 - DNA, 2325 bp, Nicotiana tabacum
  source 1..2325: mol_type = other DNA
atggccgcaactttgcacaaggtgactcctcctcctccgactactactgcttcttgctgcccttcggcttccgcttctgtcatccgtcgtgagtccgccgcagcctccgtcgtggaggacgatcagcagaaacaaacgccggcgctgacgtcattggttaactctcaacctccttcctccaatccctccggcaaagggaaacaaatcatgccaagagctcatacatgccatcctttggaccctttatctgctgctgaaatctctgtggctgtggcgaccgtcagagctgccggtgaaacacccgaggtcagagatggcatgcgctttattgaggtggttctactggaacctgataaaagtgtcattgcactggctgatgcctatttcttcccacctttccaatcttcgttgatgtccagaaggaaaggagggcttcccattcctactaagcttcctccaaggcgagctagacttattgtatataataagaaaacaaatgagacaagcatatggattgttgagctagctgaagtacatgctgctgctcgaggtggacatcacaagggaaaagtgatttcatcctatgttgttccagatgttcagccacctatagatgcacaagagtatgcagactgtgaagctgtagttaaaaattatcctccttttagggaagcaatgaagagaaggggtattgatgacatggatcttgtgatggtggacccctggtgcgttggttttcacagtgaggctgatgctcctagccgcaggcttgccaaaccgctagtattctgcagatcagagagtgactgcccaatggaaaatggatatgcaagaccggttgaaggaatttatgtacttgttgatgtgcaaaaaatgcaggtgatagagtttgaagaccgcaaacttgtacctttacctccagctgatccactgaggaattacactgctggtgagacaagaggaggggtcgatcgaagtgatgtgaaacccctccagattattcagccagagggtccaagctttcgagtcaatgggaactatgtggagtggcaaaagtggaacttccgagtaggtttcacccctagggagggtttggttatacactctgtggcatatattgacggtagtaggggtcggagacccatagcccatagattgagttttgtggagatggttgtcccctatggggatccaaatgacccacattacagaaagaacgcttttgatgcaggagaagatgggctcggaaagaatgctcattcacttaagaggggatgcgattgtttaggatacataaagtactttgatgccaattttgcaaattttaccggaggagtagaaaccactgaaaattgtgtatgtttgcatgaagaagatcacgggatgctctggaagcatcaagattggagaactggccttgcagaagttagacggtctagacgacttacagtttcttttatttgcactgtggccaattatgaatatggattctactggcacttttaccaggatgggaaaattgaagcagaaatcaaactcacaggaattctcagtttgggagcactgcctcccggagagtctcgtaaatatggcaccacaatagcaccgggattgtatgcacctgttcatcaacacttctttgttgctcgtatgaatatggcagttgattgtaaaccaggagaagcacacaatcaggttgttgaagttaatgttagagttgaagaacctgggaaagaaaatgttcacaacaatgcattctatgctaaggaaacagtgcttacgtctgaattgcaagcaatgcgtgactgtgatactttatctgctcgtcattggattgttaggaacacaagaacatccaatagaacaggacagctaacagggtacaagctggtacctggccctaactgtttgccattggctggtcctaaggctaagtttttgagaagagctgcatttttgaagcacaatctatgggttacaaaatatgcacccggagaagattttcccgggggagagttccctaatcaaaatccacgtgttggtgagggattagcttcttgggttaagcaagatcgttctctggaagaaagtgatgttgttctctggtatgtttttggaatcacacatgttcctcggttggaggactggcctgttatgccagttgaacatatcggttttatgcttcagccacatggattctttaactgctctcccgctgtagatgtacctcctcctcggggatgtgacatggaaatcaaagacagtgatggttcagaaaatggtgtagcaaagcccactcccagtaatttgatggccaagctttga

SEQ ID NO: 9 - protein, 774 aa, Nicotiana tabacum
MAATLHKVTPPPPTTTASCCPSASASVIRRESAAASVVEDDQQKQTPALTSLVNSQPPSSNPSGKGKQIMPRAHTCHPLDPLSAAEISVAVATVRAAGETPEVRDGMRFIEVVLLEPDKSVIALADAYFFPPFQSSLMSRRKGGLPIPTKLPPRRARLIVYNKKTNETSIWIVELAEVHAAARGGHHKGKVISSYVVPDVQPPIDAQEYADCEAVVKNYPPFREAMKRRGIDDMDLVMVDPWCVGFHSEADAPSRRLAKPLVFCRSESDCPMENGYARPVEGIYVLVDVQKMQVIEFEDRKLVPLPPADPLRNYTAGETRGGVDRSDVKPLQIIQPEGPSFRVNGNYVEWQKWNFRVGFTPREGLVIHSVAYIDGSRGRRPIAHRLSFVEMVVPYGDPNDPHYRKNAFDAGEDGLGKNAHSLKRGCDCLGYIKYFDANFANFTGGVETTENCVCLHEEDHGMLWKHQDWRTGLAEVRRSRRLTVSFICTVANYEYGFYWHFYQDGKIEAEIKLTGILSLGALPPGESRKYGTTIAPGLYAPVHQHFFVARMNMAVDCKPGEAHNQVVEVNVRVEEPGKENVHNNAFYAKETVLTSELQAMRDCDTLSARHWIVRNTRTSNRTGQLTGYKLVPGPNCLPLAGPKAKFLRRAAFLKHNLWVTKYAPGEDFPGGEFPNQNPRVGEGLASWVKQDRSLEESDVVLWYVFGITHVPRLEDWPVMPVEHIGFMLQPHGFFNCSPAVDVPPPRGCDMEIKDSDGSENGVAKPTPSNLMAKL

SEQ ID NO: 10 - DNA, 7018 bp, Nicotiana tabacum
  source 1..7018: mol_type = genomic DNA
atggccgcaactttgcacaaggtgactccgactactgcttcggcctccgcttctatcgcccgtcgtgagtccgccgcagcctccgtcctggtggacgatcagcagaaacaaacgccggctctgacgtcattgcttaactctcaacctccttcctccaatccctctagcaaaggtatatataaatgccttttgactctatacgtaaaattaagtttctacgctctattcaaacaatgtgtgtgtgtgtgtgttttggccggccgtcggacttcggagtatatgtatgtgtgcaattgcacaagttgttgtctctttctgtaatttgagcatctgcatgtttgattttcgtgtgcttaccgaatataaatgacggcttcgatgattttcttcatctgcattcgcaaattatttctatatgctcctaatttctatacctgtagtcttaattttggtatacattcgagaaggaaattctattgaataagctacagtagaattgcggtccttaaacatttgtttagtctcaacaatcggggtttacttttggactttgccagtgtagttgcacaattcctgcaattatttagatcaaataatatattatttcgatcactttgtcctgaatttgaattgacaatcaatcaactagttaactaattcctaatttcaaattaggtgagatcaaatcatttgtatctttgtatatccatttcgcctatgttactcggactcttcgaaaatgttgttgggtgcgtgtcggatcctacaaatttagtgcattttcggaggatccgacacgagtgcggcagcatttttggagagtccgagcaacatagcatttcgctttatactcatgtatttttctccagaggatttatactgtaatagttaaactattcttaggttgtttgtcaacctaccatgtccttccttgtttagtgaaacttaagtagatgaagttccttttgaaaagaataagaaacgatttttttcctttaaaaattaagaaaacataaaaacaaagtgtacatgaatggagggagcttgttattggagtagttggtaagatgttcattgtttgacaattccatgtttatgcagggaaacaaatcatgccaagagctcatacatgccatcctttggaccctttatctgctgctgaaatctctgtggctgtggcgaccgtcagagctgccggtgaaacacccgaggttcttaatcactatctttttgtcatccatttttgcgttgaacaatggaacaagtattagttgaacaacaggacatcatttgctctcaatttccctaattgttttcatttatttatttttttgcaaacacattttaggtcagagatggcatgcgctttattgaggtggttcttctggaacctgataaaagtgtcgttgcactggctgatgcctatttcttcccacctttccaatcttcattgatgtccagaaggaaaggagggcttcccattcctactaagcttcctccaaggcgagctagacttattgcatataataagaaaacaaatgagacaagcatatggattgttgagctagctgaagtacatgctgctgctcgaggtggacatcacaagggaaaagtgatttcatccaatgttgttccagatgttcagccacctatagtaagcactttctcgtctttagaactttaagagattagaagtacatgaacaatagatgcaattgggttatcaaagaactgaagtgagaacaccaagtaagagcaacccaaagttcttattacatttgaaaccaaatgcattcactcttcactctaataattgctcatgaaaaatgaattttaactacattttctgaaacattaccggtttcatttattggtatcctttagaatgttcacttggctagatgcaccgattaattaagttatgaacatttttgaagttgtgaatacctccatcctaatggaacatttagtaaattttcctagcggtaagttggttgttcttttgtttctatcaactggtcttgtacaactggtgctctagagattctaaacactctatgaaacaaaagagtcagaagaatttgtatttttgttgtactagcatacggtcttggggcaacgaggagaaaggacacaagccaaattggacatccattctgattccagaagccgattaagtgttttccgtgctatatatacttttgtttacttatattgctctcttcttgatgaactgtgttttctttctcatcatatggaaagtagagcatatttatacaccaaaatcagaagtaatttttgcatgtatagtctgtctcaagtcaacccaaaaaatgttaaagtttgcaacttttaagttttttggcttaagtgagttgtggtataggtcagctctttagatttcaataattctcaaattttacatattatcttaactctactgattgtaggatgcacaagagtatgctgactgtgaagctgtagttaaaaattatcctccttttagggaagcaatgaagagaaggggtattgatgacatggatgttgtgatggtggacccctggttagttaaatacccttgatttttctttcttctcccttaagaaccgatttcttatttcattgcatttcgacctgaaaatgctttccgcacaggtgcgttggttatcacagtgaggctgatgctcctagccgcaggcttgccaaaccgctagtattctgcagaacagagagtgactgcccaatggaaaatggatatgcaagaccggttgaaggaatatatgcccttgttgatgtgcaaaacatgcaggtgatagagtttgaagaccgcaaacttgtacctttacctccagctgatccactgaggaattacactgctggtgagacaagaggaggggtcgatcgaagtgatgtaaaacccctccagattattcagccagagggtccaagctttcgagtcaatgggaactatgtggaatggcaaaaggtaaattcttttttccagtctgatatgcattgaaggattatttgcttatacatctaacatgtcattctttcactttgttttgacagtggaacttccgagtaggtttcacccctagggagggtttggttatacactctgtggcatatcttgacggtagcaggggtcggagacccatagcccataggttgagttttgtggagatggttgtcccctatggggatccaaatgacccacattacagaaagaacgcttttgatgcaggagaagatgggctcggaaagaatgctcattcacttaagagggtaactgacatttctccggttcaactatggatgacttttgagaatgtaagtgctaatctaacaaaatcacccttgcagggatgcgattgtttaggatacataaagtactttgatgccaattttgcaaattttactggaggagtagaaaccactgaaaattgtgtatgtttgcatgaagaagatcacgggatgctctggaagcatcaagattggagaactggccttgcagaagttagacggtctagacgacttacagtttcttttatttgcactgtggccaattatgaatatggattctactggcacttataccaggcaagaataagaaaatcattatcaggaaatcctctgtttgcatgttctaaaaaaattaggtaacattgaagtatcatcacaagttcacagtttatttagaacacttgagggaaaagaaagcaagcagaagcaagataaagaatttcttgtaactttggtcgtgtttatgattaatgttttctgagagacaaactcattcctgggggagaaaaaaatccttattcttggccaaatgcttgaaacaaaaccaatgttatcattctgttataattgagttgataccaataatagctctactacttgcttatgccttttttttgtgggcgaggggggatttgctaagccaaagacaagtgaatatcagaagtttacatctatccccgagaaaaaaattcagaatttatgctcatctattttgcataaatgtctttgctgattgtaagtacgtttagctccacttaccattttgaaatgatctgtaaattttgtggttgcaggatgggaaaattgaagcagaagtcaaactcacaggaattctcagtttgggagcattgccccccggagagtctcgtaaatatggcaccacaatagcaccaggattgtatgcacctgttcatcaacacttctttgttgctcgtatgaatatggcagttgattgtaaaccaggagaagcacacaatcaggtatttagtggaacaagttaccgtgtccatccggaagttaaagtaatttcataccttcagtttcccaatttttaactttctaatctgagttgttagtcctttgtctaggttgttgaagttaatgtaagagttgaagaacctgggaaagaaaatgttcacaacaatgcgttctatgctaaggaaacagtgcttacgtctgaattgcaagcaatgcgggactgtgatactttatctgctcgtcattggattgtaagaagccttacttgactgtatcctcctttctttttacaggactgttcttcaggttacttccctagcacagtatctgctgagatatgtactcaatagtagactgtggtccttaaacatggccactgatttttgagggagctaagtacaatgtgtagaacctccaccttaatgctagtattctcatatgagaaatccttacaagtgtcattcttagcatttgaacaaaaattgatcgccttatttctccaaaacaatgatgttaagaactttctgtgacttgtatgtgtttggcataagagtactatatgtttcattcttctgaaaatcacgaacttttgttgagttttgcgctctttggttagatgtgtttggtgatcttaagattacaacaacaacgaaaacaacaaacccagtttagtcccacaagtgaggtttggggagggtagagtgtatgcagaccttaccccctaccttgtgaaggtagagaagttgttcccgatagacccttcgctcaaggaaagatagaaaaagaagcaatagcatcaagcagtaacaacaacacgataatagattaacagaagcgaagaaacaagaatagggataagaataagaaatagaaacaacaggtagtaaaagaaaactaaacataaggaaatgcaagagcaatactaatactactgctatggaaaagaaatacgctcaaccatacctactaaccttctaccgtaagattgcagatgaaaatttcaattgcaaattcatttagataggatggttgaactttattttgtttcagcatgtggtattgtggagtcaatttttagattccaatatctgctagctcaaaacatcaaggagaagtggaagaaattgaatactagcatccccaaccgtagtagcagagccatcgatttgactgcgaacatctgattttgcaggttaggaacacaagaacatccaatagaacaggacagctaacagggtacaagctggtacctggccctagctgtttgccattagctggtcctgaggctaagtttttgagaagagctgcatttttgaagcacaatctatgggttacacaatatgcacccggagaagattttccagggggagagttccctaatcaaaatccacgtgttggtgagggattagcttcttgggttaagcaagatcgttctctggaagaaagtgatgttgttctctggttagtttctcgacagctgtcaattatgttattacattggagttgctggtttcagattgttttgccaaacacacacactttaactttcataaagagctagatgtggaacttctaattccttggaaacctttatatacatgtttgagaagttacctattccttgaaattctgatctacacagattattacaggtatgtttttggaatcacacatgttcctcggttggaggactggcctgttatgccagttgaacatatcggttttatgcttcaggtattctggaatatggatgttatgtgtttattttgtatgaactggtgtttttctccttatctttgttatcatctaacttctcgttatctattgtgttaccttttacttggtactaaagagctgattaagtattgtatcaattttttgtaataactcaactcattcatacattcgctgtgtgtatgttagaataactagccaaacataaccttagagtgtagcattgaaagtattggcagtggagtagaaaggatcgtgaatatccacagagatcttgaaaatcaaagcaattgggagctggtagtagaaatgagaaggttctttttcaagttgttatcttgacattaagtctggatcaggttaaccacagtaagtttagtgataaaactctccatcctttttaattactgaaatctcgtaatcttttttcgaaattgctggataaactagagaattatatctcttcctcagaacctgtttaagtttattgtccaagtttagcaatgactttatccaactccaaaaacccgctggataatattcattgtgaaaactgttaagtttccggctgcctactggttaagaagattcatggaaaacttaactccacctatgtaaaattttgcatagccgatcttcaatccggataaagaagagggttgtgatatcttgacggctaaaacaataagttccctaactaatttggtattgaaacatagttgattcattgatcgatataacatggaaattcggttgaaaagggtttagttctatactcataagtcataatagaaggtggatggttccgtttgtccataacctcttcagtttaaagaacgattatttctgcttgttggtgcagccgcatggattctttaactgctctcctgctgtagatgtacctcctcctcggggatgtgacttggaaatcaaagacagtgatggttcagaaaatggtgtagcaaagcccactcccagtagtttgatggccaagctttga

SEQ ID NO: 11 - DNA, 2301 bp, Nicotiana tabacum
  source 1..2301: mol_type = other DNA
atggccgcaactttgcacaaggtgactccgactactgcttcggcctccgcttctatcgcccgtcgtgagtccgccgcagcctccgtcctggtggacgatcagcagaaacaaacgccggctctgacgtcattgcttaactctcaacctccttcctccaatccctctagcaaagggaaacaaatcatgccaagagctcatacatgccatcctttggaccctttatctgctgctgaaatctctgtggctgtggcgaccgtcagagctgccggtgaaacacccgaggtcagagatggcatgcgctttattgaggtggttcttctggaacctgataaaagtgtcgttgcactggctgatgcctatttcttcccacctttccaatcttcattgatgtccagaaggaaaggagggcttcccattcctactaagcttcctccaaggcgagctagacttattgcatataataagaaaacaaatgagacaagcatatggattgttgagctagctgaagtacatgctgctgctcgaggtggacatcacaagggaaaagtgatttcatccaatgttgttccagatgttcagccacctatagatgcacaagagtatgctgactgtgaagctgtagttaaaaattatcctccttttagggaagcaatgaagagaaggggtattgatgacatggatgttgtgatggtggacccctggtgcgttggttatcacagtgaggctgatgctcctagccgcaggcttgccaaaccgctagtattctgcagaacagagagtgactgcccaatggaaaatggatatgcaagaccggttgaaggaatatatgcccttgttgatgtgcaaaacatgcaggtgatagagtttgaagaccgcaaacttgtacctttacctccagctgatccactgaggaattacactgctggtgagacaagaggaggggtcgatcgaagtgatgtaaaacccctccagattattcagccagagggtccaagctttcgagtcaatgggaactatgtggaatggcaaaagtggaacttccgagtaggtttcacccctagggagggtttggttatacactctgtggcatatcttgacggtagcaggggtcggagacccatagcccataggttgagttttgtggagatggttgtcccctatggggatccaaatgacccacattacagaaagaacgcttttgatgcaggagaagatgggctcggaaagaatgctcattcacttaagaggggatgcgattgtttaggatacataaagtactttgatgccaattttgcaaattttactggaggagtagaaaccactgaaaattgtgtatgtttgcatgaagaagatcacgggatgctctggaagcatcaagattggagaactggccttgcagaagttagacggtctagacgacttacagtttcttttatttgcactgtggccaattatgaatatggattctactggcacttataccaggatgggaaaattgaagcagaagtcaaactcacaggaattctcagtttgggagcattgccccccggagagtctcgtaaatatggcaccacaatagcaccaggattgtatgcacctgttcatcaacacttctttgttgctcgtatgaatatggcagttgattgtaaaccaggagaagcacacaatcaggttgttgaagttaatgtaagagttgaagaacctgggaaagaaaatgttcacaacaatgcgttctatgctaaggaaacagtgcttacgtctgaattgcaagcaatgcgggactgtgatactttatctgctcgtcattggattgttaggaacacaagaacatccaatagaacaggacagctaacagggtacaagctggtacctggccctagctgtttgccattagctggtcctgaggctaagtttttgagaagagctgcatttttgaagcacaatctatgggttacacaatatgcacccggagaagattttccagggggagagttccctaatcaaaatccacgtgttggtgagggattagcttcttgggttaagcaagatcgttctctggaagaaagtgatgttgttctctggtatgtttttggaatcacacatgttcctcggttggaggactggcctgttatgccagttgaacatatcggttttatgcttcagccgcatggattctttaactgctctcctgctgtagatgtacctcctcctcggggatgtgacttggaaatcaaagacagtgatggttcagaaaatggtgtagcaaagcccactcccagtagtttgatggccaagctttga

SEQ ID NO: 12 - protein, 766 aa, Nicotiana tabacum
MAATLHKVTPTTASASASIARRESAAASVLVDDQQKQTPALTSLLNSQPPSSNPSSKGKQIMPRAHTCHPLDPLSAAEISVAVATVRAAGETPEVRDGMRFIEVVLLEPDKSVVALADAYFFPPFQSSLMSRRKGGLPIPTKLPPRRARLIAYNKKTNETSIWIVELAEVHAAARGGHHKGKVISSNVVPDVQPPIDAQEYADCEAVVKNYPPFREAMKRRGIDDMDVVMVDPWCVGYHSEADAPSRRLAKPLVFCRTESDCPMENGYARPVEGIYALVDVQNMQVIEFEDRKLVPLPPADPLRNYTAGETRGGVDRSDVKPLQIIQPEGPSFRVNGNYVEWQKWNFRVGFTPREGLVIHSVAYLDGSRGRRPIAHRLSFVEMVVPYGDPNDPHYRKNAFDAGEDGLGKNAHSLKRGCDCLGYIKYFDANFANFTGGVETTENCVCLHEEDHGMLWKHQDWRTGLAEVRRSRRLTVSFICTVANYEYGFYWHLYQDGKIEAEVKLTGILSLGALPPGESRKYGTTIAPGLYAPVHQHFFVARMNMAVDCKPGEAHNQVVEVNVRVEEPGKENVHNNAFYAKETVLTSELQAMRDCDTLSARHWIVRNTRTSNRTGQLTGYKLVPGPSCLPLAGPEAKFLRRAAFLKHNLWVTQYAPGEDFPGGEFPNQNPRVGEGLASWVKQDRSLEESDVVLWYVFGITHVPRLEDWPVMPVEHIGFMLQPHGFFNCSPAVDVPPPRGCDLEIKDSDGSENGVAKPTPSSLMAKL

SEQ ID NO: 13 - DNA, 155 bp, Nicotiana tabacum
  source 1..155: mol_type = other DNA
gagggtttagttatacactctgtggcgtatcttgatggtagcagaggtcgtagaccaatagcacataggttgagttttgtagagatggttgtcccctatggagatccaaatgatccacattataggaagaatgcatttgatgcaggagaagatgg

SEQ ID NO: 14 - DNA, 242 bp, Nicotiana tabacum
  source 1..242: mol_type = other DNA
tagaacaggacagctaacagggtacaagctggtacctggtccaaactgtttgccactggctggtcctgaggcgaaatttttgagaagagctgcatttctgaagcacaatctatgggttacacaatatgcacctggagaagattttccaggaggagagttccctaatcaaaatccccgtgttggcgagggattagcttcttgggtcaagcaagaccggcctctggaagaaagtgatattgttc

SEQ ID NO: 15 - DNA, 22 bp, Nicotiana tabacum
  source 1..22: mol_type = other DNA
atctatgggttacacaatatgc

SEQ ID NO: 16 - protein, 559 aa, Nicotiana tabacum
MFPLIILISFSLASLSETATGAVTNLSACLINHNVHNFSIYPTSRNYFNLLHFSLQNLRFAAPFMPKPTFIILPSSKEELVSTIFCCRKASYEIRVRCGGHSYEGTSYVSFDASPFVIVDLMKLDDVSVDLDSETAWAQGGATIGQIYYAIAKVSDVHAFSAGSGPTVGSGGHISGGGFGLLSRKFGLAADNVVDALLIDADGRLLDRKAMGEDVFWAIRGGGGGNWGIVYAWKIRLLKVPKIVTTCMIYRPGSKQYVAQILEKWQIVTPNLVDDFTLGVLLRPADLPADMKYGNTTPIEIFPQFNALYLGPKTEVLSISNETFPELGVKNDECKEMTWVESALFFSELADVNGNSTGDISRLKERYMDGKGFFKGKTDYVKKPVSMDGMLTFLVELEKNPKGYLVFDPYGGAMDKISDQAIAFPHRKGNLFAIQYLAQWNEEDDYMSDVYMEWIRGFYNTMTPFVSSSPRGAYINYLDMDLGVNMVDDYLLRNASSSSPSSSVDAVERARAWGEMYFLHNYDRLVKAKTQIDPLNVFRHEQSIPPMLGSTQEHKYSSE

SEQ ID NO: 17 - protein, 568 aa, Nicotiana tabacum
MFPLIILISFSLTSLSATATSGAGGGVANLYTCLIDHNVHNFSIYPTKNDQSSSNYFNLLDFSLQNLRFAASYMPKPTVIILPNSKEELVSTILCCRQASYEIRVRCGGHSYEGTSYVSFDGSPFVIVDLMKLDEVSVDLDSETAWAQGGATIGQIYYAIAKVSDVHAFSAGSGPTVGSGGHISGGGFGLLSRKFGLAADNVVDALLIDADGRLLDRKAMGEDVFWAIRGGGGGNWGIIYAWKIRLLKVPKIVTTCMIYRPGSKQYVAQLLQKWQIVTPNLVDDFTLGVLLRPADLPADMKYGNSTPIEIFPQFNALYLGPKTEVLSISNEEFPELGVKNDECKEMTWIESALFFSELADINGNSSNDISRLKERYMDGKGFFKGKTDYVKKPVSMDGMLTFLVELEKNPKGYLVFDPYGGAMDKIDDQAIAFPHRKGNLFAIQYLAQWNEEDDYKSDVYMEWIRGFYNTMTPFVSSSPRGAYINYLDMDLGVNMDDDYLLRNASSRNSSSSVDAVERARAWGEMYFLHNYDRLVKAKTQIDPLNVFRHEQSIPPMLGSTQEHKYSSE

SEQ ID NO: 18 - protein, 562 aa, Nicotiana tabacum
MFPLIILISFSFTFLSASATSGAGEGVANLSTCLINHNVHNFSMYPTSRNYFNLLDFSLQNLRFAASNMPKPTVIILPNSKEELVSTILCCRQTSYEIRVRCGGHSYEGTSSVSFDGSPFVIIDLMKLDDVSVDLDSETAWAQGGATIGQIYYAIAKASDVHAFSAGSGPTVGSGGHISGGGFGLLSRKFGVAADSVVDALLIDADGRLLDRKAMGEDVFWAIRGGGGGNWGIIYAWKIRLVKVPKIVTTFKISKPGSKQYVAPLLYKWQIVAPNLADDFTLGVQMIPIDLPADMKYGNPTPIEICPQFNGLYLGPKTEAVSILNEAFPELNVKNDDAKEMTWIESALFFSDLDNIFGNSSDDISHLKERYLGVKICFKGKSDYVKTPFSMDGIMTALVEHEKNPNAFLVFDPYGGAMDKISAQAIAFPHRKGNLFAIQYYAQWNEEDDAKSNEHIEWIRGFYNKMAPFVSSSPRGAYVNYLDMDLGMNMDDDYLLRNASSRYSSSVDAVERARAWGEKYFLNNYDRLVKAKTKIDPLNVFRHEQSIPPTLGSTQEHNYSSE

(19) United States
(12) Patent Application Publication
(10) Pub. No.: US 2025/0163443 A1
(43) Pub. Date: May 22, 2025

(54) METHODS FOR PRODUCING LOW-NICOTINE TOBACCO

(71) Applicant: NORTH CAROLINA STATE UNIVERSITY, Raleigh, NC (US)

(72) Inventors: Ralph E. DEWEY, Apex, NC (US); Ramsey S. LEWIS, Apex, NC (US)

(21) Appl. No.: 18/730,082

(22) PCT Filed: Jan. 19, 2023
(86) PCT No.: PCT/US2023/060862

§ 371 Date: Jul. 18, 2024

Related U.S. Application Data
(60) Provisional application No. 63/301,111, filed on Jan. 20, 2022.
(60) Provisional application No. 63/379,804, filed on Oct. 17, 2022.

(51) Int. Cl. C12N 15/82; A24B 13/00; A24B 15/10

(52) U.S. Cl. CPC C12N 15/8243; A24B 13/00; A24B 15/10

(57) ABSTRACT

The present technology provides methods of combining mutations in N-methylputrescine oxidase (MPO) gene isoforms with mutations in the three major berberine bridge enzyme-like (BBL) genes to produce low-nicotine tobacco plants and which prevent the accumulation of anatabine in the tobacco plants. Also provided are methods of combining these mutations to produce tobacco plants, tobacco cells, and tobacco products having reduced nicotine content and which avoid the unwanted consequence of elevated anatabine levels in the tobacco plants, cells, and products.

### STATEMENT OF PRIORITY

This application claims the benefit, under 35 U.S.C. § 119 (e), of U.S. Provisional Application No. 63/379,804 filed on Oct. 17, 2022 and U.S. Provisional Application No. 63/301,111 filed on Jan. 20, 2022, the entire contents of each of which are incorporated by reference herein.

### STATEMENT REGARDING ELECTRONIC FILING OF A SEQUENCE LISTING

A Sequence Listing in XML format, entitled 5051-1004WO_ST26.xml, 57,580 bytes in size, generated on Jan. 16, 2023 and filed herewith, is hereby incorporated by reference into the specification for its disclosures.

### TECHNICAL FIELD

The present technology relates generally to methods of combining mutations in N-methylputrescine oxidase (MPO) gene isoforms with mutations in the three major berberine bridge enzyme-like (BBL) genes to produce low-nicotine tobacco plants. The combination of mutations further prevent the accumulation of anatabine in the tobacco plants.

### BACKGROUND

The following description is provided to assist the understanding of the reader. None of the information provided or references cited are admitted to be prior art.

The pyridine alkaloids of tobacco (Nicotiana tabacum L.) are among the most studied group of plant secondary compounds in plants. Nicotine constitutes greater than 90% of the total alkaloid pool in most tobacco genotypes, and is primarily responsible for the pharmacological response experienced by users of tobacco products. In decreasing order of relative abundance, the remaining major alkaloids in tobacco include anatabine, nornicotine, and anabasine. Alkaloid levels in tobacco are influenced by environmental conditions, interactions with plant pests, and plant genetics.

Although nicotine is the primary compound that gives the users of tobacco products the pharmacological effect they seek, there are several circumstances where it would be desirable to develop products using tobacco plants that produce and accumulate very low levels of nicotine. For example, some studies have shown that the use of low-nicotine cigarettes as a component in smoking cessation strategies can help smokers who are trying to quit (Hatsukami et al., 2010a; Donny et al., 2014). Other reports have demonstrated that by lowering the nicotine levels below a critical threshold in tobacco products, they can no longer initiate or maintain an addiction response (Benowitz and Henningfield, 1994; Benowitz et al., 2007). The World Health Organization (WHO) has recommended that nicotine levels of cigarette tobacco filler be reduced to non-addictive levels of 0.4 mg/g, or below (WHO, 2015). This represents an approximate 95% reduction over that present in current cigarette tobaccos.

Tobacco alkaloid levels are also of interest because of their roles as precursors in the production of tobacco specific nitrosamines (TSNAs), a potent group of recognized carcinogens (Hecht, 1998, 2003; Hecht and Hoffman, 1989). The most problematic TSNAs are N-nitrosonornicotine (NNN) and 4-(methylnitrosamino)-1-(3-pyridyl)-1-butanone (NNK), which are derived through nitrosation reactions with nornicotine and an oxidative derivative of nicotine (such as pseudooxynicotine), respectively, during the curing, storage, and consumption of tobacco. Because tobacco alkaloids serve as essential precursors toward TSNA formation, low alkaloid tobacco plants have also been shown to produce reduced amounts of TSNAs within the cured leaf (Xie et al., 2004).

There is a need in the art for methods and compositions for modulating nicotinic alkaloid biosynthesis in plants. The present disclosure satisfies these needs.

### SUMMARY

Disclosed herein are methods and compositions for modulating nicotine biosynthesis in plants.

In one aspect, the disclosure of the present technology provides a tobacco product comprising tobacco from a Nicotiana plant, wherein the plant comprises: (A) a modification that reduces: (i) activity of BBLa, BBLb, and BBLc, or (ii) expression of a nucleic acid encoding BBLa, a nucleic acid encoding BBLb, and a nucleic acid encoding BBLc; and (B) a mutation in at least one endogenous polynucleotide selected from the group consisting of: (i) an MPO1.1 polynucleotide having about 85% to about 100% sequence identity to SEQ ID NO:1, SEQ ID NO: 2, or a polynucleotide encoding the polypeptide set forth in SEQ ID NO: 3; (ii) an MPO1.2 polynucleotide having about 85% to about 100% sequence identity to SEQ ID NO: 4, SEQ ID NO: 5, or a polynucleotide encoding the polypeptide set forth in SEQ ID NO: 6; (iii) an MPO2.1 polynucleotide having about 85% to about 100% sequence identity to SEQ ID NO: 7, SEQ ID NO: 8, or a polynucleotide encoding the polypeptide set forth in SEQ ID NO: 9; and (iv) an MPO2.2 polynucleotide having about 85% to about 100% sequence identity to SEQ ID NO: 10, SEQ ID NO: 11, or a polynucleotide encoding the polypeptide set forth in SEQ ID NO: 12, wherein the Nicotiana plant has a nicotinic alkaloid content that is reduced as compared to a plant that is not modified per (A) and does not comprise the mutations of (B).

In some embodiments, the Nicotiana plant comprises a mutation in at least two of polynucleotides (i)-(iv). In some embodiments, the Nicotiana plant comprises a mutation in at least three of polynucleotides (i)-(iv) In some embodiments, the Nicotiana plant comprises a mutation in each of polynucleotides (i)-(iv).

In some embodiments, the nicotinic alkaloid is nicotine. In some embodiments, the Nicotiana plant comprises a nicotine content of about 0.4 mg/g or less. In some embodiments, the Nicotiana plant comprises a nicotine content of about 0.1 mg/g or less.

In some embodiments, the combination of modifications of (A) and the mutation of (B) has a synergistic effect in the reduction of nicotine content in the Nicotiana plant. In some embodiments, the synergistic effect comprises a reduction of nicotine content in the Nicotiana plant that is greater than that resulting from either the modifications of (A) alone or the mutation of (B) alone.

In some embodiments, the Nicotiana plant comprises an anatabine content that is reduced as compared to a plant that is not modified per (A) but does comprise the mutation of (B).

In some embodiments, the tobacco is selected from the group consisting of leaf tobacco, shredded tobacco, cut tobacco, ground tobacco, powder tobacco, tobacco extract, smokeless tobacco, moist or dry snuff, pipe tobacco, cigar tobacco, cigarillo tobacco, cigarette tobacco, and chewing tobacco.

In some embodiments, the product is selected from the group consisting of a cigarillo, a kretek cigarette, a non-ventilated recess filter cigarette, a vented recess filter cigarette, a cigar, snuff, tobacco-containing gum, tobacco-containing lozenges, and chewing tobacco.

In some embodiments, the Nicotiana plant is a Nicotiana tabacum plant.

In one aspect, the disclosure of the present technology provides a method of producing a Nicotiana plant having reduced nicotinic alkaloid content, comprising combining in a Nicotiana plant: (A) a modification that reduces: (i) activity of BBLa, BBLb, and BBLc or (ii) expression of a nucleic acid encoding BBLa, a nucleic acid encoding BBLb, and a nucleic acid encoding BBLc; and (B) a mutation in at least one endogenous polynucleotide selected from the group consisting of: (i) an MPO1.1 polynucleotide having about 85% to about 100% sequence identity to SEQ ID NO:1, SEQ ID NO: 2, or a polynucleotide encoding the polypeptide set forth in SEQ ID NO: 3; (ii) an MPO1.2 polynucleotide having about 85% to about 100% sequence identity to SEQ ID NO: 4, SEQ ID NO: 5, or a polynucleotide encoding the polypeptide set forth in SEQ ID NO: 6; (iii) an MPO2.1 polynucleotide having about 85% to about 100% sequence identity to SEQ ID NO: 7, SEQ ID NO: 8, or a polynucleotide encoding the polypeptide set forth in SEQ ID NO: 9; and (iv) an MPO2.2 polynucleotide having about 85% to about 100% sequence identity to SEQ ID NO: 10, SEQ ID NO: 11, or a polynucleotide encoding the polypeptide set forth in SEQ ID NO: 12, wherein the Nicotiana plant has a nicotinic alkaloid content that is reduced as compared to a plant that is not modified per (A) and does not comprise the mutations of (B).

In some embodiments, the Nicotiana plant comprises a mutation in at least two of polynucleotides (i)-(iv). In some embodiments, the Nicotiana plant comprises a mutation in at least three of polynucleotides (i)-(iv). In some embodiments, the Nicotiana plant comprises a mutation in each of polynucleotides (i)-(iv).

In some embodiments, generation of the mutation comprises introducing into the plant at least one RNAi plasmid that suppresses expression of a gene product encoded by one or more of polynucleotides (i)-(iv). In some embodiments, the at least one RNAi plasmid comprises at least 21 consecutive nucleotides of the nucleic acid sequence set forth in SEQ ID NO: 13 or SEQ ID NO: 14 in sense and/or antisense orientation.

In some embodiments, generation of the mutation comprises introducing into the plant at least one recombinant nucleic acid encoding a nuclease targeting at least one of polynucleotides (i)-(iv). In some embodiments, generation of the mutation comprises introducing into the plant at least one recombinant nucleic acid encoding a nuclease targeting at least two of polynucleotides (i)-(iv). In some embodiments, generation of the mutation comprises introducing into the plant at least one recombinant nucleic acid encoding a nuclease targeting at least three of polynucleotides (i)-(iv). In some embodiments, generation of the mutation comprises introducing into the plant at least one recombinant nucleic acid encoding a nuclease targeting each of polynucleotides (i)-(iv).

In some embodiments, the nuclease comprises a meganuclease, a zinc finger nuclease (ZFN), a transcription activator-like effector nuclease (TALEN), and/or a clustered regularly interspaced short palindromic repeat (CRISPR) associated (Cas) nuclease. In some embodiments, the nuclease comprises a meganuclease. In some embodiments, the meganuclease is designed to recognize a target sequence comprising a 15-40 base-pair cleavage site in at least one of polynucleotides (i)-(iv). In some embodiments, the meganuclease is designed to recognize a target sequence comprising a 5′-ATCTATGGGTTACACAATATGC-3′ (SEQ ID NO: 15). In some embodiments, the mutation is a deletion or an insertion.

In some embodiments, the nicotinic alkaloid is nicotine. In some embodiments, the Nicotiana plant comprises a nicotine content of about 0.4 mg/g or less. In some embodiments, the Nicotiana plant comprises a nicotine content of about 0.1 mg/g or less.

In some embodiments, the combination of modifications per (A) and the mutation of (B) has a synergistic effect in the reduction of nicotine content in the Nicotiana plant. In some embodiments, the synergistic effect comprises a reduction of nicotine content in the Nicotiana plant that is greater than that resulting from either the modifications of (A) alone or the mutation of (B) alone.

In some embodiments, the Nicotiana plant comprises an anatabine content that is reduced as compared to a plant that is not modified per (A) but does comprise the mutations of (B).

In some embodiments, the present technology provides a Nicotiana plant produced by any one of the methods, wherein the plant comprises: (A) a modification that reduces: (i) activity of BBLa, BBLb, and BBLc or (ii) expression of a nucleic acid encoding BBLa, a nucleic acid encoding BBLb, and a nucleic acid encoding BBLc; and (B) a mutation in at least one endogenous polynucleotide selected from the group consisting of: (i) an MPO1.1 polynucleotide having about 85% to about 100% sequence identity to SEQ ID NO:1, SEQ ID NO: 2, or a polynucleotide encoding the polypeptide set forth in SEQ ID NO: 3; (ii) an MPO1.2 polynucleotide having about 85% to about 100% sequence identity to SEQ ID NO: 4, SEQ ID NO: 5, or a polynucleotide encoding the polypeptide set forth in SEQ ID NO: 6; (iii) an MPO2.1 polynucleotide having about 85% to about 100% sequence identity to SEQ ID NO: 7, SEQ ID NO: 8, or a polynucleotide encoding the polypeptide set forth in SEQ ID NO: 9; and (iv) an MPO2.2 polynucleotide having about 85% to about 100% sequence identity to SEQ ID NO: 10, SEQ ID NO: 11, or a polynucleotide encoding the polypeptide set forth in SEQ ID NO: 12.

In some embodiments, the plant is characterized by decreased nicotine content and decreased anatabine content as compared to a plant that is not modified per (A) and does not comprise the mutations of (B).

In some embodiments, the present technology provides a progeny plant or seed produced from the Nicotiana plant of claim 34 or claim 35, wherein the progeny plant or seed comprises: (A) a modification that reduces (i) activity of BBLa, BBLb, and BBLc or (ii) expression of a nucleic acid encoding BBLa, a nucleic acid encoding BBLb, and a nucleic acid encoding BBLc; and (B) a mutation in at least one endogenous polynucleotide selected from the group consisting of: (i) an MPO1.1 polynucleotide having about 85% to about 100% sequence identity to SEQ ID NO:1, SEQ ID NO: 2, or a polynucleotide encoding the polypeptide set forth in SEQ ID NO: 3; (ii) an MPO1.2 polynucleotide having about 85% to about 100% sequence identity to SEQ ID NO: 4, SEQ ID NO: 5, or a polynucleotide encoding the polypeptide set forth in SEQ ID NO: 6; (iii) an MPO2.1 polynucleotide having about 85% to about 100% sequence identity to SEQ ID NO: 7, SEQ ID NO: 8, or a polynucleotide encoding the polypeptide set forth in SEQ ID NO: 9; and (iv) an MPO2.2 polynucleotide having about 85% to about 100% sequence identity to SEQ ID NO: 10, SEQ ID NO: 11, or a polynucleotide encoding the polypeptide set forth in SEQ ID NO: 12.

In some embodiments, the Nicotiana plant is a Nicotiana tabacum plant.

In one aspect, the disclosure of the present technology provides a method of producing a Nicotiana plant cell having reduced nicotinic alkaloid content, comprising combining in a Nicotiana plant cell: (A) a modification that reduces: (i) activity of BBLa, BBLb, and BBLc or (ii) expression of a nucleic acid encoding BBLa, a nucleic acid encoding BBLb, and a nucleic acid encoding BBLc; and (B) a mutation in at least one endogenous polynucleotide selected from the group consisting of: (i) an MPO1.1 polynucleotide having about 85% to about 100% sequence identity to SEQ ID NO:1, SEQ ID NO: 2, or a polynucleotide encoding the polypeptide set forth in SEQ ID NO: 3; (ii) an MPO1.2 polynucleotide having about 85% to about 100% sequence identity to SEQ ID NO: 4, SEQ ID NO: 5, or a polynucleotide encoding the polypeptide set forth in SEQ ID NO: 6; (iii) an MPO2.1 polynucleotide having about 85% to about 100% sequence identity to SEQ ID NO: 7, SEQ ID NO: 8, or a polynucleotide encoding the polypeptide set forth in SEQ ID NO: 9; and (iv) an MPO2.2 polynucleotide having about 85% to about 100% sequence identity to SEQ ID NO: 10, SEQ ID NO: 11, or a polynucleotide encoding the polypeptide set forth in SEQ ID NO: 12, wherein the Nicotiana plant cell has a nicotinic alkaloid content that is reduced as compared to a plant cell that is not modified per (A) and does not comprise the mutations of (B).

In some embodiments, the Nicotiana plant cell comprises a mutation in at least two of polynucleotides (i)-(iv). In some embodiments, the Nicotiana plant cell comprises a mutation in at least three of polynucleotides (i)-(iv). In some embodiments, the Nicotiana plant cell comprises a mutation in each of polynucleotides (i)-(iv).

In some embodiments, generation of the mutation comprises introducing into the plant cell at least one RNAi plasmid that suppresses expression of a gene product encoded by one or more of polynucleotides (i)-(iv). In some embodiments, the at least one RNAi plasmid comprises at least 21 consecutive nucleotides of the nucleic acid sequence set forth in SEQ ID NO: 13 or SEQ ID NO: 14 in sense and/or antisense orientation.

In some embodiments, generation of the mutation comprises introducing into the plant cell at least one recombinant nucleic acid encoding a nuclease targeting at least one of polynucleotides (i)-(iv). In some embodiments, generation of the mutation comprises introducing into the plant cell at least one recombinant nucleic acid encoding a nuclease targeting at least two of polynucleotides (i)-(iv). In some embodiments, generation of the mutation comprises introducing into the plant cell at least one recombinant nucleic acid encoding a nuclease targeting at least three of polynucleotides (i)-(iv). In some embodiments, generation of the mutation comprises introducing into the plant cell at least one recombinant nucleic acid encoding a nuclease targeting each of polynucleotides (i)-(iv).

In some embodiments, the nuclease comprises a meganuclease, a zinc finger nuclease (ZFN), a transcription activator-like effector nuclease (TALEN), and/or a clustered regularly interspaced short palindromic repeat (CRISPR) associated (Cas) nuclease. In some embodiments, the nuclease comprises a meganuclease. In some embodiments, the meganuclease is designed to recognize a target sequence comprising a 15-40 base-pair cleavage site in at least one of polynucleotides (i)-(iv). In some embodiments, the meganuclease is designed to recognize a target sequence comprising a 5′-ATCTATGGGTTACACAATATGC-3′ (SEQ ID NO: 15). In some embodiments, the mutation is a deletion or an insertion.

In some embodiments, the nicotinic alkaloid is nicotine. In some embodiments, the Nicotiana plant cell comprises a nicotine content of about 0.4 mg/g or less. In some embodiments, the Nicotiana plant cell comprises a nicotine content of about 0.1 mg/g or less.

In some embodiments, the combination of modifications per (A) and the mutation of (B) has a synergistic effect in the reduction of nicotine content in the Nicotiana plant cell. In some embodiments, the synergistic effect comprises a reduction of nicotine content in the Nicotiana plant cell that is greater than that resulting from either the modifications of (A) alone or the mutation of (B) alone.

In some embodiments, the Nicotiana plant cell comprises an anatabine content that is reduced as compared to a plant cell that is not modified per (A) but does comprise the mutations of (B).

In some embodiments, the technology of the present disclosure provides a Nicotiana plant comprising the Nicotiana plant cell produced by the methods, wherein the plant comprises: (A) a modification that reduces: (i) activity of BBLa, BBLb, and BBLc or (ii) expression of a nucleic acid encoding BBLa, a nucleic acid encoding BBLb, and a nucleic acid encoding BBLc; and (B) a mutation in at least one endogenous polynucleotide selected from the group consisting of: (i) an MPO1.1 polynucleotide having about 85% to about 100% sequence identity to SEQ ID NO:1, SEQ ID NO: 2, or a polynucleotide encoding the polypeptide set forth in SEQ ID NO: 3; (ii) an MPO1.2 polynucleotide having about 85% to about 100% sequence identity to SEQ ID NO: 4, SEQ ID NO: 5, or a polynucleotide encoding the polypeptide set forth in SEQ ID NO: 6; (iii) an MPO2.1 polynucleotide having about 85% to about 100% sequence identity to SEQ ID NO: 7, SEQ ID NO: 8, or a polynucleotide encoding the polypeptide set forth in SEQ ID NO: 9; and (iv) an MPO2.2 polynucleotide having about 85% to about 100% sequence identity to SEQ ID NO: 10, SEQ ID NO: 11, or a polynucleotide encoding the polypeptide set forth in SEQ ID NO: 12.

In some embodiments, the plant is characterized by decreased nicotine content and decreased anatabine content as compared to a plant that is not modified per (A) and does not comprise the mutations of (B).

In some embodiments, the technology of the present disclosure provides a progeny plant or seed produced from the Nicotiana plant, wherein the progeny plant or seed comprises: (A) a modification that reduces (i) activity of BBLa, BBLb, and BBLc or (ii) expression of a nucleic acid encoding BBLa, a nucleic acid encoding BBLb, and a nucleic acid encoding BBLc; and (B) a mutation in at least one endogenous polynucleotide selected from the group consisting of: (i) an MPO1.1 polynucleotide having about 85% to about 100% sequence identity to SEQ ID NO:1, SEQ ID NO: 2, or a polynucleotide encoding the polypeptide set forth in SEQ ID NO: 3; (ii) an MPO1.2 polynucleotide having about 85% to about 100% sequence identity to SEQ ID NO: 4, SEQ ID NO: 5, or a polynucleotide encoding the polypeptide set forth in SEQ ID NO: 6; (iii) an MPO2.1 polynucleotide having about 85% to about 100% sequence identity to SEQ ID NO: 7, SEQ ID NO: 8, or a polynucleotide encoding the polypeptide set forth in SEQ ID NO: 9; and (iv) an MPO2.2 polynucleotide having about 85% to about 100% sequence identity to SEQ ID NO: 10, SEQ ID NO: 11, or a polynucleotide encoding the polypeptide set forth in SEQ ID NO: 12.

In some embodiments, the Nicotiana plant is a Nicotiana tabacum plant.

Both the foregoing summary and the following description of the drawings and detailed description are exemplary and explanatory. They are intended to provide further details of the disclosure, but are not to be construed as limiting. Other objects, advantages, and novel features will be readily apparent to those skilled in the art from the following detailed description of the disclosure.

### BRIEF DESCRIPTION OF THE DRAWINGS

FIG. 1 is an abbreviated diagram of the alkaloid biosynthetic pathway in tobacco (adapted from Dewey and Xie, 2013). A622: isoflavone reductase-like protein; ADC: arginine decarboxylase; BBL: berberine bridge enzyme-like; MPO: N-methylputrescine oxidase; NND: nicotine N-demethylase; ODC: ornithine decarboxylase; PMT: putrescine methyltransferase; QPT: quinolate phosphoribosyltransferase. Enzymatic steps whose substrates and end products have not been fully characterized are indicated with a question mark.

FIGS. 2A-2H are a set of charts showing alkaloid means for groups of primary transformants carrying functioning (+) or non-functioning (−) MPO RNAi transgene insertions. FIGS. 2A, 2C, 2E, and 2G are charts showing RNAi suppression of MPO in a wild type K326 background. FIGS. 2B, 2D, 2F, and 2H are charts showing RNAi suppression of MPO in a K326 line, designated K326 222, that contains EMS-mediated knockout mutations in BBLa, BBLb, and BBLc. Standard errors are shown. P values for t-tests between group means are provided for each group pair. Number of individuals per group is indicated within the bars for the anabasine determinations.

FIGS. 3A-3H are a set of charts showing alkaloid means of T1 generation plants possessing ARCUS® meganuclease-induced mutations in MPO genes. FIGS. 3A, 3C, 3E, and 3G are charts showing MPO mutant analysis in the flue-cured lines K326 (K11 and K23) and K326 222 (KB19 and KB20). FIGS. 3B, 3D, 3F, and 3H are charts showing MPO mutant analysis in the burley lines TN90 (T23) and TN90 222 (TB7). The MPO mutations present in the lines represented on the x-axis are defined in Table 1 and Table 2. Standard errors are shown. Means±standard errors of means with the same letter are not significantly different at a 5% significance level using Tukey-Kramer HSD grouping. BLOQ=below level of quantification. Number of individuals per group is indicated within the bars for the anabasine determinations.

FIGS. 4A and 4B are charts showing the aerial biomass of plants containing mutations in BBL and/or MPO genes. The results from plants in the K326 and TN90 backgrounds are shown in FIGS. 4A and 4B, respectively. FIG. 4A shows the aerial biomass in the flue cured lines K326 WT, K326 bbla/b/c (also referred to herein as “K326 222”), K11, KB19 and KB20. FIG. 4B shows the aerial biomass in the burley lines TN90 LC (a TN90 WT control), TN90 bbla/b/c (also referred to herein as “TN90 222”), T23, and TB7. The MPO mutations present in the lines represented on the x-axis are defined in Table 1 and Table 2. Means±standard errors of means with the same letter are not significantly different at a 5% significance level using Tukey-Kramer HSD grouping. Number of individuals per group ranged from 25-30 for the lines in K326, and from 17-22 for the lines in TN90.

FIGS. 4C-4J are a set of charts showing the alkaloid mean (mg/g) of plants containing mutations in BBL and/or MPO genes. FIGS. 4C (nicotine content), 4E (nornicotine content), 4G (anatabine content), and 4I (anabasine content) are charts showing alkaloid analysis in the flue-cured lines K326 WT, K326 bbla/b/c (also referred to herein as “K326 222”), K11, KB19, and KB20. FIGS. 4D (nicotine content), 4F (nornicotine content), 4H (anatabine content), and 4J (anabasine content) are charts showing alkaloid analysis in the burley lines TN90 LC, TN90 bbla/b/c (also referred to as “TN90 222”), T23 and TB7. The MPO mutations present in the lines represented on the x-axis are defined in Table 1 and Table 2. Means±standard errors of means with the same letter are not significantly different at a 5% significance level using Tukey-Kramer HSD grouping. Number of individuals per group ranged from 25-30 for the lines in K326, and from 17-22 for the lines in TN90.

### DETAILED DESCRIPTION

### I. Introduction

The disclosure of the present technology relates to methods for downregulating or completely knocking out N-methylputrescine oxidase (MPO) gene function in tobacco plants comprising mutations in the three major berberine bridge enzyme-like (BBL) genes, BBLa, BBLb, and BBLc, to reduce the levels of alkaloids, such as nicotine, in tobacco plants including commercial grade tobacco cultivars and avoid the unwanted consequence of elevated anatabine levels in the tobacco plants. In particular, it was surprisingly discovered that the undesirable increase in anatabine levels that accompanies MPO gene suppression can be avoided by downregulating or knocking out specific MPO isoforms in a combination with mutations in BBLa, BBLb, and BBLc. This is desirable as downregulating MPO gene function can be used to make low nicotine products, which are becoming highly desirable. For example, on Dec. 23, 2021, the US Food and Drug Administration (FDA) authorized the marketing of low nicotine combustible cigarettes which have “95% less nicotine” and “greatly reduces nicotine consumption.” (www.fda.gov/news-events/press-announcements/fda-authorizes-marketing-tobacco-products-help-reduce-exposure-and-consumption-nicotine-smokers-who).

As mentioned above, one unwanted consequence of inhibiting MPO gene function is that the production and accumulation of the pyridine alkaloid anatabine becomes elevated in these plants to a level that greatly exceeds that which is observed in normal tobaccos. As demonstrated herein, two important advantages can be realized by pyramiding MPO gene disruption with mutations in BBLa, BBLb and BBLc. First, the elevated anatabine phenotype conferred by inhibiting MPO function is eliminated; and second, the nicotine content is lowered below that which is attainable by implementing the MPO and BBL disruption technologies alone.

In particular, and as detailed in Example 3, disclosed herein is support for the advantages of combining the triple mutant bbl (bbla/b/c) trait with the mutant mpo technology as a strategy for producing tobaccos that accumulate ultra-low levels of not only nicotine, but also of all four of the major tobacco alkaloids (nicotine, nornicotine, anatabine and anabasine). Although knocking out BBL and MPO gene function alone mediated nicotine reductions in the range of 88-93%, each of the individual technologies alone suffered the drawback of causing other alkaloids to become elevated beyond that which was observed in the parental cultivar: mutations in BBL genes alone led to a greater than 2-fold increase in nornicotine, and mutations in MPO genes alone were associated with a greater than 4-fold increase in anatabine and a 1.6- to 1.8-fold increase in anabasine. Lines with mutations in both BBL and MPO genes, however, not only displayed a dramatically reduced nicotine content phenotype (99% or greater), but they were also significantly lower in all of the alkaloid species measured.

Accordingly, these results demonstrate that combining MPO mutations with BBL mutations in tobacco plants is useful in methods for producing tobacco plants characterized by significantly reduced nicotine levels and lacking the accumulation of nornicotine, anatabine and anabasine, as compared to wild-type control tobacco plants and/or plants in which the MPO or BBL mutations alone are present.

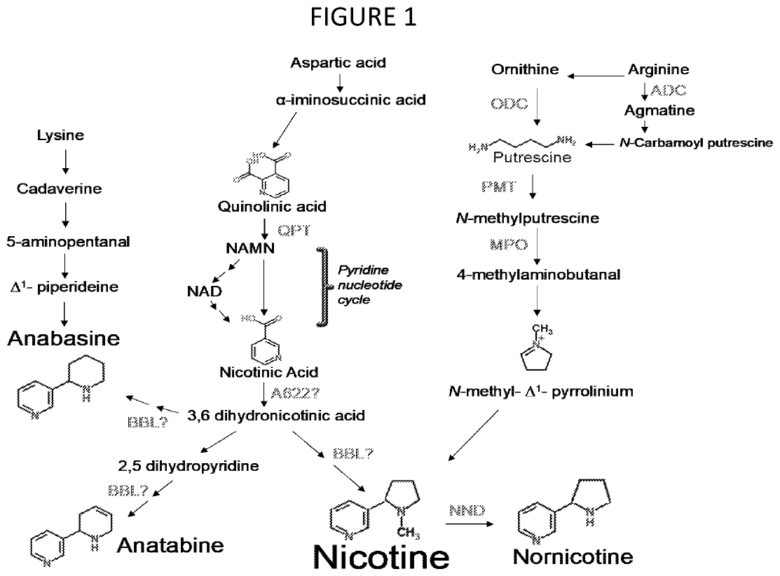

A great deal of knowledge has been gained over the past few decades regarding the molecular genetics underlying the biosynthesis of tobacco alkaloids. FIG. 1 shows an abbreviated version of the alkaloid biosynthetic pathway of tobacco. The enzyme methylputrescine oxidase (MPO) catalyzes the oxidative deamination of N-methylputrescine to produce 4-methylaminobutanal, a compound that spontaneously cyclizes to form the N-methyl-Δ-pyrrolinium ring that becomes incorporated into nicotine by an enzymatic step(s) that has yet to be clearly defined (FIG. 1).

The MPO enzyme is encoded by two closely related isoforms, designated MPO1.1 and MPO1.2 by Kajikawa et al. (2017). MPO1.1 originated from the N. sylvestris ancestor of N. tabacum, whereas as MPO1.2 originated from the N. tomentosiformis-like progenitor species. In vitro assays of recombinant MPO1.1 enzyme produced in E. coli were used to demonstrate enzyme function. These assays showed that MPO1.1 utilized N-methylputrescine as a substrate more efficiently than any other diamine tested (Heim et al., 2007; Katoh et al., 2007). The tobacco genome also contains another set of closely related genes, designated MPO2.1 and MPO2.2. The predicted protein products of MPO1 enzymes share 88% amino acid identity and 96% homology to their MPO2 counterparts (Katoh et al., 2007). Subsequent studies revealed that MPO2 enzymes preferentially accept non-N-methylated amines over their corresponding N-methylated versions. This observation, coupled with transcript analyses that showed MPO1 genes to be coordinately regulated with alkaloid biosynthetic genes and highly expressed in the root, in contrast to MPO2 transcripts that are expressed at low levels constitutively throughout the plant, prompted investigators change the nomenclature of MPO2 to the more generalized term diamine oxidase (DAO) 1 (Naconsie et al., 2014). Like MPO1, DAO1 genes are present in the genome as two closely related isoforms: DAO1.1 which originated from N. sylvestris and DAO1.2 that originated from the N. tomentosiformis-like ancestor (Kajikawa et al., 2017). The genomic sequence of MPO1.1 is set forth in SEQ ID NO: 1, the cDNA sequence of MPO1.1 is set forth in SEQ ID NO: 2, and the predicted protein sequence of MPO1.1 is set forth in SEQ ID NO: 3. The genomic sequence of MPO1.2 is set forth in SEQ ID NO: 4, the cDNA sequence of MPO1.2 is set forth in SEQ ID NO: 5, and the predicted protein sequence of MPO1.2 is set forth in SEQ ID NO: 6. The genomic sequence of MPO2.1 (DAO1.1) is set forth in SEQ ID NO: 7, the cDNA sequence of MPO2.1 is set forth in SEQ ID NO: 8, and the predicted protein sequence of MPO2.1 is set forth in SEQ ID NO: 9. The genomic sequence of MPO2.2 (DAO1.2) is set forth in SEQ ID NO: 10, the cDNA sequence of MPO2.2 is set forth in SEQ ID NO: 11, and the predicted protein sequence of MPO2.2 is set forth in SEQ ID NO: 12.

When anti-MPO RNAi constructs were introduced into tobacco hairy root cultures, a 93-99% reductions in nicotine were accompanied by 5- to 10-fold increases in anatabine content, making this normally low abundance species the predominant alkaloid in these materials (Shoji and Hashimoto, 2008). Similarly, BY-2 culture cells, which are naturally low in MPO gene activity, also produce anatabine as the most abundant alkaloid species. In addition to the MPO gene family, RNAi suppression of ODC and PMT genes also results in low nicotine plants with exceptionally high anatabine levels (Chintapakorn and Hamill, 2003; Wang et al., 2009; DeBoer et al., 2011; Dalton et al., 2016). As shown in FIG. 1, these three gene families encode enzymes responsible for the synthesis of the pyrrolidine ring of nicotine. Of the four most abundant alkaloids in tobacco, anatabine is the only one that solely utilizes the pyridine ring pathway for the synthesis of both of its ringed structures. It has been proposed that inhibition of ODC, PMT, and MPO expression results in a reduction in the pool of pyrrolidine ring precursors required for nicotine (and nornicotine) synthesis. The insufficient supply of pyrrolidine rings, in turn, is believed to lead to an overaccumulation of pyridine ring precursors, which by default become incorporated into anatabine (Dewey and Xie, 2013).

### II. Nicotine and Related Compounds in Tobacco

The pyridine alkaloids of tobacco (Nicotiana tabacum L.) are among the most studied group of plant secondary compounds in plants. Nicotine constitutes greater than 90% of the total alkaloid pool in most tobacco genotypes, and is primarily responsible for the pharmacological response experienced by users of tobacco products. In decreasing order of relative abundance, the remaining major alkaloids in tobacco include anatabine, nornicotine, and anabasine. Alkaloid levels in tobacco are influenced by environmental conditions, interactions with plant pests, and plant genetics.

Although nicotine is the primary compound that gives the users of tobacco products the pharmacological effect they seek, there are several circumstances where it would be desirable to develop products using tobacco plants that produce and accumulate very low levels of nicotine. For example, some studies have shown that the use of low-nicotine cigarettes as a component in smoking cessation strategies can help smokers who are trying to quit (Hatsukami et al., 2010a; Donny et al., 2014). Other reports have demonstrated that by lowering the nicotine levels below a critical threshold in tobacco products, they can no longer initiate or maintain an addiction response (Benowitz and Henningfield, 1994; Benowitz et al., 2007). Studies such as these may ultimately influence regulatory agencies, such as the U.S. Food and Drug Administration, that have been given the authority to determine what acceptable levels of various tobacco constituents (including nicotine) will be allowable in cigarettes and other tobacco products. The World Health Organization (WHO) has recommended that nicotine levels of cigarette tobacco filler be reduced to non-addictive levels of 0.4 mg/g, or below (WHO, 2015). This represents an approximate 95% reduction over that present in current cigarette tobaccos.

Tobacco alkaloid levels are also of interest because of their roles as precursors in the production of tobacco specific nitrosamines (TSNAs), a potent group of recognized carcinogens (Hecht, 1998, 2003; Hecht and Hoffman, 1989). The most problematic TSNAs are N-nitrosonornicotine (NNN) and 4-(methylnitrosamino)-1-(3-pyridyl)-1-butanone (NNK), which are derived through nitrosation reactions with nornicotine and an oxidative derivative of nicotine (such as pseudooxynicotine), respectively, during the curing, storage, and consumption of tobacco. Because tobacco alkaloids serve as essential precursors toward TSNA formation, low alkaloid tobacco plants have also been shown to produce reduced amounts of TSNAs within the cured leaf (Xie et al., 2004).

### III. Role of MPO in Alkaloid Biosynthesis

A great deal of knowledge has been gained over the past few decades regarding the molecular genetics underlying the biosynthesis of tobacco alkaloids. FIG. 1 shows an abbreviated version of the alkaloid biosynthetic pathway of tobacco. The enzyme methylputrescine oxidase (MPO) catalyzes the oxidative deamination of N-methylputrescine to produce 4-methylaminobutanal, a compound that spontaneously cyclizes to form the N-methyl-Δ-pyrrolinium ring that becomes incorporated into nicotine by an enzymatic step(s) that has yet to be clearly defined (FIG. 1).

The MPO enzyme is encoded by two closely related isoforms, designated MPO1.1 and MPO1.2 by Kajikawa et al. (2017). MPO1.1 originated from the N. sylvestris ancestor of N. tabacum, whereas as MPO1.2 originated from the N. tomentosiformis-like progenitor species. In vitro assays of recombinant MPO1.1 enzyme produced in E. coli were used to demonstrate enzyme function. These assays showed that MPO1.1 utilized N-methylputrescine as a substrate more efficiently than any other diamine tested (Heim et al., 2007; Katoh et al., 2007). The tobacco genome also contains another set of closely related genes, designated MPO2.1 and MPO2.2. The predicted protein products of MPO1 enzymes share 88% amino acid identity and 96% homology to their MPO2 counterparts (Katoh et al., 2007). Subsequent studies revealed that MPO2 enzymes preferentially accept non-N-methylated amines over their corresponding N-methylated versions. This observation, coupled with transcript analyses that showed MPO1 genes to be coordinately regulated with alkaloid biosynthetic genes and highly expressed in the root, in contrast to MPO2 transcripts that are expressed at low levels constitutively throughout the plant, prompted investigators change the nomenclature of MPO2 to the more generalized term diamine oxidase (DAO) 1 (Naconsie et al., 2014).

Like MPO1, DAO1 genes are present in the genome as two closely related isoforms: DAO1.1 which originated from N. sylvestris and DAO1.2 that originated from the N. tomentosiformis-like ancestor (Kajikawa et al., 2017). The genomic sequence of MPO1.1 is set forth in SEQ ID NO: 1, the cDNA sequence of MPO1.1 is set forth in SEQ ID NO: 2, and the predicted protein sequence of MPO1.1 is set forth in SEQ ID NO: 3. The genomic sequence of MPO1.2 is set forth in SEQ ID NO: 4, the cDNA sequence of MPO1.2 is set forth in SEQ ID NO: 5, and the predicted protein sequence of MPO1.2 is set forth in SEQ ID NO: 6. The genomic sequence of MPO2.1 (DAO1.1) is set forth in SEQ ID NO: 7, the cDNA sequence of MPO2.1 is set forth in SEQ ID NO: 8, and the predicted protein sequence of MPO2.1 is set forth in SEQ ID NO: 9. The genomic sequence of MPO2.2 (DAO1.2) is set forth in SEQ ID NO: 10, the cDNA sequence of MPO2.2 is set forth in SEQ ID NO: 11, and the predicted protein sequence of MPO2.2 is set forth in SEQ ID NO: 12.

When anti-MPO RNAi constructs were introduced into tobacco hairy root cultures, 93-99% reductions in nicotine were accompanied by 5- to 10-fold increases in anatabine content, making this normally low abundance species the predominant alkaloid in these materials (Shoji and Hashimoto, 2008). Similarly, BY-2 culture cells which are naturally low in MPO gene activity also produce anatabine as the most abundant alkaloid species. In addition to the MPO gene family, RNAi suppression of ODC and PMT genes also results in low nicotine plants with exceptionally high anatabine levels (Chintapakorn and Hamill, 2003; Wang et al., 2009; DeBoer et al., 2011; Dalton et al., 2016). As shown in FIG. 1, these three gene families encode enzymes responsible for the synthesis of the pyrrolidine ring of nicotine. Of the four most abundant alkaloids in tobacco, anatabine is the only one that solely utilizes the pyridine ring pathway for the synthesis of both of its ringed structures.

It has been proposed that inhibition of ODC, PMT, and MPO expression results in a reduction in the pool of pyrrolidine ring precursors required for nicotine (and nornicotine) synthesis. The insufficient supply of pyrrolidine rings, in turn, is believed to lead to an overaccumulation of pyridine ring precursors, which by default become incorporated into anatabine (Dewey and Xie, 2013).

### IV. Development of Low Nicotine Tobaccos Through the Inhibition of MPO Gene Function

Although suppression of MPO expression has been shown to reduce the nicotine content of hairy root cultures and BY-2 cell cultures (Shoji and Hashimoto, 2008), studies that report the effects of inhibiting MPO gene function in the leaves of whole tobacco plants are unknown. To investigate this, as described herein, the inventors of the present technology used two separate strategies to determine the consequences of impairing MPO gene activity in intact plants: (1) RNAi-mediated degradation of MPO transcripts; and (2) targeted mutagenesis of MPO genes using the ARCUS® genome editing technology of Precision Biosciences (precisionbiosciences.com). In addition to assessing the effects of disrupting MPO gene function alone, the inventors tested whether combining MPO gene inhibition with the separate alkaloid reduction technology mediated by mutations in the three major BBL genes (Lewis et al., 2015; 2020) could result in additional reductions in leaf alkaloid content. As described below, the skilled artisan would understand that MPO and BBL gene activity could be modulated in a number of ways generally known in the art.

### V. Modulating Alkaloid Production in Plants

The disclosure of the present technology relates to methods for downregulating or completely knocking out N-methylputrescine oxidase (MPO) gene function in tobacco plants comprising mutations in the three major berberine bridge enzyme-like (BBL) genes, BBLa, BBLb, and BBLc, to reduce the levels of alkaloids, such as nicotine, in tobacco plants including commercial grade tobacco cultivars and avoid the unwanted consequence of elevated anatabine levels in the tobacco plants.

Alkaloid production may be reduced by suppression of any one or more endogenous genes encoding MPO1.1, MPO1.2, MPO2.1, or MPO2.2 in a number of ways generally known in the art, for example, RNA interference (RNAi) techniques, artificial microRNA techniques, virus-induced gene silencing (VIGS) techniques, antisense techniques, sense co-suppression techniques, targeted mutagenesis techniques, and genome editing or targeted genome engineering techniques (e.g., the CRISPR (clustered regularly interspaced short palindromic repeats)/Cas (CRISPR-associated) system, meganucleases, zinc finger nucleases (ZFNs), and TAL effector nucleases (TALENs)). Accordingly, the present technology provides methodology and constructs for decreasing alkaloid content in a plant by suppressing any one or more of MPO1.1, MPO1.2, MPO2.1, or MPO2.2.

In some embodiments, Nicotiana plants produced by the methods of the present technology comprising both mpo and bbl mutations may comprise a nicotinic alkaloid content that is reduced by at least 40% (e.g., at least about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99%, or more; or any range or value therein) as compared to a wild-type plant that does not comprise the mpo and bbl mutations. In some embodiments, the nicotinic alkaloid that is reduced in the Nicotiana plant produced by the methods of the present technology may be nicotine, wherein the nicotine content may be reduced by about 40% to about 99% (e.g., at least about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99%, or more; or any range or value therein) as compared to a wild-type plant that does not comprise the mpo and bbl mutations. In some embodiments, the nicotinic alkaloid that is reduced in the Nicotiana plant produced by the methods of the present technology may be anatabine, wherein the anatabine content may be reduced by about 40% to about 90% (e.g., at least about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90%, or more; or any range or value therein) as compared to a wild-type plant that does not comprise the mpo and bbl mutations.

### VI. Genetic Engineering of Plants and Cells

### A. MPO Isoform Sequences Nucleic Acid Constructs

MPO1.1 genes described herein include the sequence set forth in SEQ ID NO: 1 (MPO1.1 genomic sequence), including biologically active fragments thereof of at least about 15 contiguous nucleic acids up to about 5471 contiguous nucleic acids, or any value of contiguous nucleic acids in between these two amounts. MPO1.1 genes described herein also include the sequence set forth in SEQ ID NO: 2 (MPO1.1 cDNA sequence), including biologically active fragments thereof of at least about 15 contiguous nucleic acids up to about 2373 contiguous nucleic acids, or any value of contiguous nucleic acids in between these two amounts.

MPO1.2 genes described herein include the sequence set forth in SEQ ID NO: 4 (MPO1.2 genomic sequence), including biologically active fragments thereof of at least about 15 contiguous nucleic acids up to about 6541 contiguous nucleic acids, or any value of contiguous nucleic acids in between these two amounts. MPO1.2 genes described herein also include the sequence set forth in SEQ ID NO: 5 (MPO1.2 cDNA sequence), including biologically active fragments thereof of at least about 15 contiguous nucleic acids up to about 2361 contiguous nucleic acids, or any value of contiguous nucleic acids in between these two amounts.

MPO2.1 (DAO1.1) genes described herein include the sequence set forth in SEQ ID NO: 7 (MPO2.1 genomic sequence), including biologically active fragments thereof of at least about 15 contiguous nucleic acids up to about 7565 contiguous nucleic acids, or any value of contiguous nucleic acids in between these two amounts. MPO2.1 genes described herein also include the sequence set forth in SEQ ID NO: 8 (MPO2.1 cDNA sequence), including biologically active fragments thereof of at least about 15 contiguous nucleic acids up to about 2325 contiguous nucleic acids, or any value of contiguous nucleic acids in between these two amounts.

MPO2.2 (DAO1.2) genes described herein include the sequence set forth in SEQ ID NO: 10 (MPO2.2 genomic sequence), including biologically active fragments thereof of at least about 15 contiguous nucleic acids up to about 7018 contiguous nucleic acids, or any value of contiguous nucleic acids in between these two amounts. MPO2.2 genes described herein also include the sequence set forth in SEQ ID NO: 11 (MPO2.2 cDNA sequence), including biologically active fragments thereof of at least about 15 contiguous nucleic acids up to about 2301 contiguous nucleic acids, or any value of contiguous nucleic acids in between these two amounts.

In some embodiments, MPO1.1, MPO1.2, MPO2.1, and MPO2.2 genes of the present technology include the sequences set forth in SEQ ID NOs: 1, 2, 4, 5, 7, 8, 10, and 11 including biologically active fragments thereof of at least about 21 consecutive nucleotides, which are of a sufficient length as to be useful in induction of gene silencing in plants (Hamilton & Baulcombe, Science, 286:950-952 (1999)).

The present technology also includes “variants” of SEQ ID NOs: 1, 2, 4, 5, 7, 8, 10, and 11 with one or more bases deleted, substituted, inserted, or added, which variant codes for a polypeptide that regulates alkaloid biosynthesis activity. Accordingly, sequences having “base sequences with one or more bases deleted, substituted, inserted, or added” retain physiological activity even when the encoded amino acid sequence has one or more amino acids substituted, deleted, inserted, or added.

For example, the poly A tail or 5′- or 3′-end, nontranslated regions may be deleted, and bases may be deleted to the extent that amino acids are deleted. Bases may also be substituted, as long as no frame shift results. Bases also may be “added” to the extent that amino acids are added.

Unless otherwise indicated, all nucleotide sequences determined by sequencing a DNA molecule herein were determined using an automated DNA sequencer, such as the Model 3730xl from Applied Biosystems, Inc. Therefore, as is known in the art for any DNA sequence determined by this automated approach, any nucleotide sequence determined herein may contain some errors. Nucleotide sequences determined by automation are typically at least about 95% identical, more typically at least about 96% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence may be completely different from the amino acid sequence actually encoded by the sequenced DNA molecule, beginning at the point of such an insertion or deletion.

For purposes of the present technology, two sequences hybridize under stringent conditions when they form a double-stranded complex in a hybridization solution of 6×SSE, 0.5% SDS, 5×Denhardt's solution and 100 μg of non-specific carrier DNA. See Ausubel, et al., supra, at section 2.9, supplement 27 (1994). Sequences may hybridize at “moderate stringency,” which is defined as a temperature of 60° C. in a hybridization solution of 6×SSE, 0.5% SDS, 5×Denhardt's solution and 100 μg of non-specific carrier DNA. For “high stringency” hybridization, the temperature is increased to 68° C. Following the moderate stringency hybridization reaction, the nucleotides are washed in a solution of 2×SSE plus 0.05% SDS for five times at room temperature, with subsequent washes with 0.1×SSC plus 0.1% SOS at 60° C. for 1 h. For high stringency, the wash temperature is increased to 68° C. For the purpose of the technology, hybridized nucleotides are those that are detected using 1 ng of a radiolabeled probe having a specific radioactivity of 10,000 cpm/ng, where the hybridized nucleotides are clearly visible following exposure to X-ray film at −70° C. for no more than 72 hours.

The present technology encompasses nucleic acid molecules which are at least about 90%, about 91%, about 92%, about 93%, about 94%, about 95%, about 96%, about 97%, about 98%, about 99% or 100% identical to a nucleic acid sequence described in SEQ ID NO: 1, 2, 4, 5, 7, 8, 10, 11, 13, 14, and 15. Differences between two nucleic acid sequences may occur at the 5′ or 3′ terminal positions of the reference nucleotide sequence or anywhere between those terminal positions, interspersed either individually among nucleotides in the reference sequence or in one or more contiguous groups within the reference sequence.

The present technology also encompasses amino acid molecules which are at least about 90%, about 91%, about 92%, about 93%, about 94%, about 95%, about 96%, about 97%, about 98%, about 99% or 100% identical to a nucleic acid sequence described in SEQ ID NO: 3, 6, 9, and 12. Differences between two amino acid sequences may occur at the N- or C-terminal positions of the reference amino acid sequence or anywhere between those terminal positions, interspersed either individually among amino acids in the reference sequence or in one or more contiguous groups within the reference sequence.

### B. Nucleic Acid Constructs

Recombinant nucleic acid constructs may be made using standard techniques. For example, the DNA sequence for transcription may be obtained by treating a vector containing the sequence with restriction enzymes to cut out the appropriate segment. The DNA sequence for transcription may also be generated by annealing and ligating synthetic oligonucleotides or by using synthetic oligonucleotides in a polymerase chain reaction (PCR) to give suitable restriction sites at each end. The DNA sequence then is cloned into a vector containing suitable regulatory elements, such as upstream promoter and downstream terminator sequences.

Promoters useful for expression of a nucleic acid sequence introduced into a cell to either decrease or increase expression of a gene that regulates alkaloid biosynthesis may be constitutive promoters, such as the carnation etched ring virus (CERV), cauliflower mosaic virus (CaMV) 35S promoter, or more particularly the double enhanced cauliflower mosaic virus promoter, comprising two CaMV 35S promoters in tandem (referred to as a “Double 35S” promoter). In some embodiments, the promoter is a Glycine Max Ubiquitin 3 (GmUBI3) gene promoter. Tissue-specific, tissue-preferred, cell type-specific, and inducible promoters may be desirable under certain circumstances. For example, a tissue-specific promoter allows for overexpression in certain tissues without affecting expression in other tissues.

Additional exemplary promoters include promoters which are active in root tissues, such as the tobacco RB7 promoter (see, e.g., Hsu et al., Pestic. Sci. 44:9-19 (1995); U.S. Pat. No. 5,459,252), maize promoter CRWAQ81 (see, e.g., U.S. Patent Publication No. 2005/0097633); the Arabidopsis ARSK1 promoter (see, e.g., Hwang & Goodman, Plant J. 8:37-43 (1995)), the maize MR7 promoter (see, e.g., U.S. Pat. No. 5,837,848), the maize ZRP2 promoter (see, e.g., U.S. Pat. No. 5,633,363), the maize MTL promoter (see, e.g., U.S. Pat. Nos. 5,466,785 and 6,018,099) the maize MRS1, MRS2, MRS3, and MRS4 promoters (see, e.g., U.S. Patent Publication No. 2005/0010974), and an Arabidopsis cryptic promoter (see, e.g., U.S. Patent Publication No. 2003/0106105).

The vectors of the present technology may also contain termination sequences, which are positioned downstream of the nucleic acid molecules of the present technology, such that transcription of mRNA is terminated, and polyA sequences added. Exemplary terminators include Agrobacterium tumefaciens nopaline synthase terminator (Tnos), Agrobacterium tumefaciens mannopine synthase terminator (Tmas), and the CaMV 35S terminator (T35S). Termination regions include the pea ribulose bisphosphate carboxylase small subunit termination region (TrbcS) or the Tnos termination region. The expression vector also may contain enhancers, start codons, splicing signal sequences, and targeting sequences.

Expression vectors of the present technology may also contain a selection marker by which transformed cells can be identified in culture. The marker may be associated with the heterologous nucleic acid molecule, i.e., the gene operably linked to a promoter. As used herein, the term “marker” refers to a gene encoding a trait or a phenotype that permits the selection of, or the screening for, a plant or cell containing the marker. In plants, for example, the marker gene will encode antibiotic or herbicide resistance. This allows for selection of transformed cells from among cells that are not transformed or transfected.

Examples of suitable selectable markers include but are not limited to adenosine deaminase, dihydrofolate reductase, hygromycin-B-phosphotransferase, thymidine kinase, xanthine-guanine phospho-ribosyltransferase, glyphosate and glufosinate resistance, and amino-glycoside 3-O-phosphotransferase (kanamycin, neomycin and G418 resistance). These markers may include resistance to G418, hygromycin, bleomycin, kanamycin, and gentamicin. The construct may also contain the selectable marker gene bar that confers resistance to herbicidal phosphinothricin analogs like ammonium gluphosinate. See, e.g., Thompson et al., EMBO J. 9:2519-23 (1987)). Other suitable selection markers known in the art may also be used.

Visible markers such as green florescent protein (GFP) may be used. Methods for identifying or selecting transformed plants based on the control of cell division have also been described. See, e.g., WO 2000/052168 and WO 2001/059086.

Replication sequences, of bacterial or viral origin, may also be included to allow the vector to be cloned in a bacterial or phage host. Preferably, a broad host range prokaryotic origin of replication is used. A selectable marker for bacteria may be included to allow selection of bacterial cells bearing the desired construct. Suitable prokaryotic selectable markers also include resistance to antibiotics such as kanamycin or tetracycline.

Other nucleic acid sequences encoding additional functions may also be present in the vector, as is known in the art. For example, when Agrobacterium is the host, T-DNA sequences may be included to facilitate the subsequent transfer to and incorporation into plant chromosomes.

Such gene constructs may suitably be screened for activity by transformation into a host plant via Agrobacterium and screening for modified alkaloid levels.

Suitably, the nucleotide sequences for the genes may be extracted from the GenBank™ nucleotide database and searched for restriction enzymes that do not cut. These restriction sites may be added to the genes by conventional methods such as incorporating these sites in PCR primers or by sub-cloning.

Constructs may be comprised within a vector, such as an expression vector adapted for expression in an appropriate host (plant) cell. It will be appreciated that any vector which is capable of producing a plant comprising the introduced DNA sequence will be sufficient.

Suitable vectors are well known to those skilled in the art and are described in general technical references such as Pouwels et al., Cloning Vectors, A Laboratory Manual, Elsevier, Amsterdam (1986). Examples of suitable vectors include the Ti plasmid vectors.

In some embodiments, the present technology provides vectors that enable the suppression of MPO1.1, MPO1.2, MPO2.1, and/or MPO2.2, and/or BBLa, BBLb, and/or BBLc, and for modulating the production levels of nicotine and other alkaloids. These vectors can be transiently introduced into host plant cells or stably integrated into the genomes of host plant cells to generate transgenic plants by various methods known to persons skilled in the art.

C. Methodology for Suppressing Genes that Regulate Alkaloid Production

In some embodiments of the present technology, methods and constructs are provided for suppressing MPO gene isoforms and BBLabc genes that regulate alkaloid production, altering alkaloid levels, and producing plants with altered alkaloid levels. Examples of methods that may be used for suppressing an MPO or BBLabc gene that regulates alkaloid production (e.g., MPO1.1, MPO1.2, MPO2.1, MPO2.2, BBLa, BBLb, BBLc) include antisense, sense co-suppression, RNAi, artificial microRNA, virus-induced gene silencing (VIGS), antisense, sense co-suppression, and targeted mutagenesis.

RNAi and Artificial microRNA

RNAi techniques involve stable transformation using RNAi plasmid constructs (Helliwell & Waterhouse, Methods Enzymol. 392:24-35 (2005)). Such plasmids are composed of a fragment of the target gene to be silenced in an inverted repeat structure. The inverted repeats are separated by a spacer, often an intron. The RNAi construct driven by a suitable promoter, for example, the Cauliflower mosaic virus (CaMV) 35S promoter, is integrated into the plant genome and subsequent transcription of the transgene leads to an RNA molecule that folds back on itself to form a double-stranded hairpin RNA. This double-stranded RNA structure is recognized by the plant endonucleases known as Dicer that cut the double-stranded RNAs into small RNAs (about 21 nucleotides long) called small interfering RNAs (siRNAs). siRNAs associate with the RNA Induced Silencing Complex (RISC) which goes on to direct degradation of the mRNA for the target gene.

Artificial microRNA (amiRNA) techniques exploit the microRNA (miRNA) pathway that functions to silence endogenous genes in plants and other eukaryotes (Schwab et al., Plant Cell 18:1121-33 (2006); Alvarez et al., Plant Cell 18:1134-51 (2006)). In this method, 21-nucleotide-long fragments of the gene to be silenced are introduced into a pre-miRNA gene to form a pre-amiRNA construct. The pre-miRNA construct is transferred into the plant genome using transformation methods apparent to one skilled in the art. After transcription of the pre-amiRNA, processing yields amiRNAs that target genes, which share nucleotide identity with the 21 nucleotide amiRNA sequence.

### Virus-Induced Gene Silencing (VIGS)

Virus-induced gene silencing (VIGS) techniques are a variation of RNAi techniques that exploits the endogenous-antiviral defenses of plants. Infection of plants with recombinant VIGS viruses containing fragments of host DNA leads to post-transcriptional gene silencing for the target gene. In one embodiment, a tobacco rattle virus (TRV) based VIGS system can be used. Tobacco rattle virus based VIGS systems are described for example, in Baulcombe, Curr. Opin. Plant Biol. 2:109-113 (1999); Lu et al., Methods 30:296-303 (2003); Ratcliff et al., The Plant Journal 25:237-245 (2001); and U.S. Pat. No. 7,229,829.

### Antisense and Sense Techniques

Antisense techniques involve introducing into a plant an antisense oligonucleotide that will bind to the messenger RNA (mRNA) produced by the gene of interest. The “antisense” oligonucleotide has a base sequence complementary to the gene's messenger RNA (mRNA), which is called the “sense” sequence. Activity of the sense segment of the mRNA is blocked by the anti-sense mRNA segment, thereby effectively inactivating gene expression. Application of antisense to gene silencing in plants is described in more detail in Stam et al., Plant J. 21 27-42 (2000).

Sense co-suppression techniques involve introducing a highly expressed sense transgene into a plant resulting in reduced expression of both the transgene and the endogenous gene (Depicker and van Montagu, Curr. Opin. Cell Biol. 9: 373-82 (1997)). The effect depends on sequence identity between transgene and endogenous gene.

### Targeted Mutagenesis

Targeted mutagenesis techniques, for example TILLING (Targeting Induced Local Lesions IN Genomes) and “delete-a-gene” using fast-neutron bombardment, may be used to knockout gene function in a plant (Henikoff et al., Plant Physiol. 135: 630-6 (2004); Li et al., Plant J. 27: 235-242 (2001)). TILLING involves treating seeds or individual cells with a mutagen to cause point mutations that are then discovered in genes of interest using a sensitive method for single-nucleotide mutation detection. Detection of desired mutations (e.g., mutations resulting in the inactivation of the gene product of interest) may be accomplished, for example, by PCR methods. For example, oligonucleotide primers derived from the gene of interest may be prepared and PCR may be used to amplify regions of the gene of interest from plants in the mutagenized population. Amplified mutant genes may be annealed to wild-type genes to find mismatches between the mutant genes and wild-type genes. Detected differences may be traced back to the plants which had the mutant gene thereby revealing which mutagenized plants will have the desired expression (e.g. silencing of the gene of interest). These plants may then be selectively bred to produce a population having the desired expression. TILLING can provide an allelic series that includes missense and knockout mutations, which exhibit reduced expression of the targeted gene. TILLING is touted as a possible approach to gene knockout that does not involve introduction of transgenes, and therefore may be more acceptable to consumers. Fast-neutron bombardment induces mutations, i.e., deletions, in plant genomes that can also be detected using PCR in a manner similar to TILLING.

### D. Targeted Genome Engineering of Plants and Cells to Reduce Expression of Endogenous Nicotine Biosynthesis Genes

### Meganucleases

In some embodiments, the compositions and methods described herein employ a meganuclease DNA binding domain for binding to a region of interest in the genome of a plant cell. Meganucleases are engineered versions of naturally occurring restriction enzymes that typically have extended DNA recognition sequences (e.g., about 14 to about 40 base pairs in length). Meganucleases (also known as homing endonucleases) are commonly grouped into five families based on sequence and structure motifs: the LAGLIDADG family, the GIY-YIG family, the His-Cyst box family, the PD-(D/E)XK family, and the HNH family. In some embodiments, the meganuclease comprises an engineered homing endonuclease. The recognition sequences of homing endonucleases and meganucleases such as I-Sce, I-CeuI, PI-PspI, PI-Sce, I-SceIV, I-CsmI, I-PanI, I-SceII, I-PpoI, I-SceIII, I-CreI, I-TevI, I-TevII, and I-TevIII are known. I-CreI is a member of the LAGLIDADG family of homing endonucleases which recognizes and cuts a 22 basepair recognition sequence in the chloroplast chromosome of the algae Chlamydomonas reinhardtii. Genetic selection techniques have been used to modify the wild-type I-CreI cleavage site preference (Sussman et al. (2004), J. Mol. Biol. 342: 31-41; Chames et al. (2005), Nucleic Acids Res. 33: e178; Seligman et al. (2002), Nucleic Acids Res. 30: 3870-9, Arnould et al. (2006), J. Mol. Biol. 355: 443-58). Methods of rationally-designing mono-LAGLIDADG homing endonucleases have been described, which are capable of comprehensively redesigning I-CreI and other homing endonucleases to target widely-divergent DNA sites, including sites in mammalian, yeast, plant, bacterial, and viral genomes (WO 2007/047859).

In some embodiments, the meganuclease is tailored or designed to recognize a target site in a nicotine biosynthesis gene. In some embodiments, the meganuclease is tailored or designed to recognize a target in one or more of an MPO1.1, MPO1.2, MPO2.1, MPO2.2, BBLa, BBLb, and BBLc gene. The meganucleases as described herein may bind to and/or cleave the region of interest in a region upstream of the coding region of the gene. Gene insertion or correction can be achieved by the introduction of a DNA repair matrix containing sequences homologous to the endogenous sequence surrounding the DNA break. Mutations can be created either at or distal to the break. In some embodiments, the meganuclease generates a specific sequence change in the 5′-UTR of a nicotine biosynthesis gene, such as generating a single nucleotide mutation to form an out-of-frame start codon upstream of the gene's ORF.

### CRISPR Cas System

In some embodiments, the methods of the present technology relate to the use of a CRISPR/Cas system that binds to a target site in a region of interest in a genome, wherein the CRISPR/Cas system comprises a CRISPR/Cas nuclease and an engineered crRNA/tracrRNA (or single guide RNA (sgRNA) or guide RNA (gRNA)). In some embodiments, the CRISPR system generally comprises (i) a polynucleotide encoding a Cas protein, and (ii) at least one sgRNA for RNA-guided genome engineering in plant cells.

Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Cys3, Cse1, Cse2, Csc1, Csc2, Csa5, Csn2, Csm2, Csm3, Csm4, Csm5, Csm6, Smr1, Cmr3, Cmr4, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. In some embodiments, the Cas protein is a Streptococcus pyogenes Cas9 protein. These enzymes are known. For example, the amino acid sequence of S. pyogenes Cas9 protein may be found in the SwissProt database under accession number Q99ZW2.

The sgRNA molecules comprise a crRNA-tracrRNA scaffold polynucleotide and a targeting sequence corresponding to a genomic target of interest.

In some embodiments, the CRISPR/Cas system recognizes a target site in a nicotine biosynthesis gene. In some embodiments, the CRISPR/Cas system recognizes a target in one or more of an MPO1.1, MPO1.2, MPO2.1, MPO2.2, BBLa, BBLb, and BBLc gene. The CRISPR/Cas system as described herein may bind to and/or cleave the region of interest in a region upstream of the coding region of a nicotine biosynthesis gene. In some embodiments, the CRISPR/Cas system generates a specific sequence change in the 5′-UTR of a nicotine biosynthesis gene, such as generating a single nucleotide mutation to form an out-of-frame start codon upstream of the gene's ORF.

The CRISPR/Cas system is based on the Cas9 nuclease and an engineered single guide RNA (sgRNA) that specifies the targeted nucleic acid sequence. Cas9 is a large monomeric DNA nuclease guided to a DNA target sequence adjacent to the PAM (protospacer adjacent motif) sequence motif by a complex of two non-coding RNAs: CRISPR RNA (crRNA) and trans-activating crRNA (tacrRNA).

The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can induce site-specific double strand breaks (DSBs) into genomic DNA of live cells. See, e.g., Mussolino, Nat. Biotechnol., 31:208-209 (2013). In some embodiments, the Cas9 protein is expressed in a plant cell as a fusion to a nuclear localization signal (NLS) to ensure delivery into nuclei. In some embodiments, the Cas9 protein is tagged (e.g., FLAG- or GFP-tagged). In some embodiments, promoters (e.g., Cauliflower mosaic virus 35S) may be used to drive Cas9 expression in a plant cell. In some embodiments, the Cas9 enzyme is S. pneumoniae, S. pyogenes, or S. thermophiles Cas9, and may include mutated Cas9 derived from these organisms. The enzyme may be a Cas9 homolog or ortholog. In some embodiments, the CRISPR enzyme is codon-optimized for expression in a plant cell, such as a Nicotiana tabacum cell.

The single guide RNA (sgRNA) is the second component of the CRISPR/Cas system that forms a complex with the Cas9 nuclease. The sgRNA is created by fusing crRNA with tacrRNA. The sgRNA guide sequence located at the 5′ end confers DNA target specificity. By modifying the guide sequence, sgRNAs with different target specificities can be designed to target any desired endogenous gene. In some embodiments, the target sequence is about 1 to about 10 base pairs upstream of the transcription start site (e.g., positions −10, −9, −8, −7, −6, −5, −4, −3, −2, or −1). It is not intended that the present technology be limited to any particular distance restraint with regard to the location of the guide RNA target sequence from the gene transcription start site. In some embodiments, the target sequence lies “in proximity to” a gene of interest, where “in proximity to” refers to any distance from the gene of interest, wherein the Cas9-regulatory domain fusion is able to exert an effect on gene expression. In some embodiments, the target sequence lies upstream of the ORF of the gene of interest.

The canonical length of the guide sequence is about 20 bp and the DNA target sequence is about 20 bp followed by a PAM sequence having the consensus NGG sequence. In some embodiments, sgRNAs are expressed in a plant cell using plant RNA polymerase III promoters, such as U6 and U3.

When the DSBs are repaired by either NHEJ or HDR, the sequence at the repair site can be modified or new genetic information can be inserted (e.g., donor DNA comprising a desired mutation can be inserted into the target gene at the break site). Although HDR typically occurs at lower and more variable frequencies than NHEJ, it can be leveraged to generate precise, defined modifications at a target locus in the presence of an exogenously introduced repair template. Accordingly, exogenous repair templates, designed by methods known in the art, can also be delivered into a cell, most often in the form of a synthetic, single-stranded DNA donor oligo or DNA donor plasmid, to generate a precise change in the genome. Single-stranded DNA donor oligos are delivered into a cell to insert or change short sequences (SNPs, amino acid substitutions, epitope tags, etc.) of DNA in the endogenous genomic target region. The benefits of using a synthetic DNA donor oligo is that no cloning is required to generate the donor template and DNA modifications can be added during synthesis for different applications, such as increased resistance to nucleases. Traditionally, the maximum insert length recommended for use with a DNA donor oligo is about 50 nucleotides.

In some embodiments, the present technology provides an engineered, programmable, non-naturally occurring CRISPR/Cas system comprising a Cas9 protein and one or more single guide RNAs (sgRNAs) that target the genomic loci of DNA molecules encoding one or more gene products in the nicotine biosynthesis pathway and the Cas9 protein cleaves the genomic loci of the DNA molecules encoding the one or more gene products, whereby expression of the one or more gene products is altered. In some embodiments, Cas9 introduces multiple DSBs in the same cell (i.e., multiplexes) via expression of one or more distinct guide RNAs.

In some embodiments, the present technology provides a method for targeted genomic modification of plant cells to alter the expression of at least one of MPO1.1, MPO1.2, MPO2.1, MPO2.2, BBLa, BBLb, and BBLc, the method comprising introducing into a plant cell, comprising and expressing a DNA molecule having a target sequence and encoding the nicotine biosynthesis gene, an engineered CRISPR/Cas system comprising (a) an expression construct comprising a first polynucleotide encoding a bacterial Cas9 protein, or a variant thereof or a fusion protein therewith, and a second polynucleotide encoding a guide RNA comprising: (i) a crRNA-tracrRNA scaffold polynucleotide, and (ii) a targeting sequence operably linked to the crRNA-tracrRNA scaffold polynucleotide, where the targeting sequence corresponds to a genomic locus of interest, and (b) delivering the expression construct into the plant cell, where the first and second polynucleotides are expressed (transcribed) within the plant cell. This method can optionally further include visualizing, identifying, or selecting for plant cells having a genomic modification at the genomic locus of interest that is induced by the delivering the expression construct into the plant cell.

In some embodiments of the methods of the present technology, the Cas9 polypeptide and guide RNA are encoded on two separate vectors. In these methods, the steps generally follow the sequence of introducing into a plant cell containing and expressing a DNA molecule having a target sequence and encoding the nicotine biosynthesis gene an engineered CRISPR/Cas system comprising (a) a Cas9 polynucleotide or a conservative variant thereof, and a guide RNA comprising (i) a crRNA-tracrRNA scaffold polynucleotide, and (ii) a targeting sequence operably linked to the crRNA-tracrRNA scaffold polynucleotide, with the targeting sequence corresponding to a genomic locus of interest, and (b) delivering the two polynucleotides into the plant cell. In variations of this method, a donor polynucleotide having homology to the genomic target of interest is included in a cotransfection. In some variations of these methods, the transfected material can be either plasmid DNA or RNA generated by in vitro transcription. In still other variations, the methods for targeted genomic modification are multiplexed, meaning that more than one genomic locus is targeted for modification. In still other variations of these methods, the transformation of the plant cells can be followed by visualizing, identifying, or selecting for plant cells having a genomic modification at the genomic locus of interest.

### TALENs

In some embodiments, the compositions and methods described herein employ transcription activator-like effector nucleases (TALENs) to edit plant genomes by inducing double-strand breaks (DSBs). TALENs are restriction enzymes that can be engineered to cleave specific sequences of DNA. TALENs are constructed by fusing a TAL effector DNA-binding domain to a DNA cleavage domain (e.g., a nuclease domain such as that derived from the FokI endonuclease). Transcription activator-like effectors (TALEs) can be engineered according to methods known in the art to bind to a desired DNA sequence, and when combined with a nuclease, provide a technique for cutting DNA at specific locations. For example, after a target sequence in a nicotine biosynthesis gene is identified, a corresponding TALEN sequence is engineered and inserted into a plasmid. The plasmid is inserted into a target cell where it is translated to produce a functional TALEN, which then enters the nucleus where it binds to and cleaves its target sequence. Such an approach can be employed to introduce an exogenous DNA sequence into the target gene as the DSB is being repaired through either homology-directed repair or non-homologous end-joining. For example, in some embodiments, the use of TALEN technology generates a specific sequence change (e.g., insertion, deletion, or substitution) in the 5′-UTR of a nicotine biosynthesis gene, resulting in the production of an out-of-frame start codon upstream of the gene's ORF.

### ZFNs

In some embodiments, the compositions and methods described herein employ zinc finger nucleases (ZFNs) to edit plant genomes by inducing double-strand breaks (DSBs). ZFNs are artificial restriction enzymes generated by fusing a zinc finder DNA-binding domain to a DNA cleavage domain (e.g., a nuclease domain such as that derived from the FokI endonuclease). ZFNs can be engineered to bind and cleave DNA at specific locations. ZFNs contain two protein domains. The first domain is the DNA-binding domain, which contains eukaryotic transcription factors and the zinc finger. The second domain is a nuclease domain that contains the FokI restriction enzyme responsible for cleaving DNA. ZFNs can be engineered according to methods known in the art to bind to a desired DNA sequence and cleave DNA at specific locations. For example, after a target sequence in a nicotine biosynthesis gene is identified, a corresponding ZFN sequence is engineered and inserted into a plasmid. The plasmid is inserted into a target cell where it is translated to produce a functional ZFN, which then enters the nucleus where it binds to and cleaves its target sequence introducing a double strand break (DSB). Such an approach can be employed to introduce an exogenous DNA sequence into the target gene as the DSB is being repaired through either homology-directed repair or non-homologous end-joining. For example, in some embodiments, the use of ZFN technology generates a specific sequence change in the 5′-UTR of a nicotine biosynthesis gene, such as the insertion of an out-of-frame start codon upstream of the gene's ORF.

### E. Host Plants and Cells

In some embodiments, the present technology relates to the genetic manipulation of a plant or cell. Accordingly, the present technology provides methodology and constructs for reducing alkaloid synthesis in a plant.

The plants utilized in the present technology may include the class of alkaloid-producing higher plants amenable to genetic engineering techniques, including both monocotyledonous and dicotyledonous plants, as well as gymnosperms. In some embodiments, the alkaloid-producing plant includes a nicotinic alkaloid-producing plant of the Nicotiana, Duboisia, Solanum, Anthocercis, and Salpiglossis genera in the Solanaceae or the Eclipta and Zinnia genera in the Compositae.

As known in the art, there are a number of ways by which genes and gene constructs can be introduced into plants, and a combination of plant transformation and tissue culture techniques have been successfully integrated into effective strategies for creating transgenic crop plants.

These methods, which can be used in the present technology, have been described elsewhere (Potrykus, Annu. Rev. PlantPhysiol. Plant Mol. Biol. 42:205-225 (1991); Vasil, Plant Mol. Biol. 5:925-937 (1994); Walden and Wingender, Trends Biotechnol. 13:324-331 (1995); Songstad et al., Plant Cell, Tissue and Organ Culture 40:1-15 (1995)), and are well known to persons skilled in the art. For example, one skilled in the art will certainly be aware that, in addition to Agrobacterium-mediated transformation of Arabidopsis by vacuum infiltration (Bechtold et al., C. R. Acad. Sci. Ser. III Sci. Vie, 316:1194-1199 (1993)) or wound inoculation (Katavic et al., Mol. Gen. Genet. 245:363-370 (1994)), it is equally possible to transform other plant and crop species, using Agrobacterium Ti-plasmid-mediated transformation (e.g., hypocotyl (DeBlock et al., Plant Physiol. 91:694-701 (1989)) or cotyledonary petiole (Moloney et al., Plant Cell Rep. 8:238-242 (1989) wound infection), particle bombardment/biolistic methods (Sanford et al., J. Part. Sci. Technol. 5:27-37 (1987); Nehra et al., Plant J. 5: 285-297 (1994); Becker et al., Plant J. 5: 299-307 (1994)), or polyethylene glycol-assisted protoplast transformation (Rhodes et al., Science 240: 204-207 (1988); Shimamoto et al., Nature 335: 274-276 (1989)) methods.

Agrobacterium rhizogenes may be used to produce transgenic hairy roots cultures of plants, including tobacco, as described, for example, by Guillon et al., Curr. Opin. Plant Biol. 9:341-6 (2006). “Tobacco hairy roots” refers to tobacco roots that have T-DNA from an Ri plasmid of Agrobacterium rhizogenes integrated in the genome and grow in culture without supplementation of auxin and other phytohormones. Tobacco hairy roots produce nicotinic alkaloids as roots of a whole tobacco plant do.

Additionally, plants may be transformed by Rhizobium, Sinorhizobium, or Mesorhizobium transformation. (Broothaerts et al., Nature 433:629-633 (2005)).

After transformation of the plant cells or plant, those plant cells or plants into which the desired DNA has been incorporated may be assessed for zygosity and selected by such methods as antibiotic resistance, herbicide resistance, tolerance to amino-acid analogues or using phenotypic markers (See, e.g., Passricha et al., J. Biol. Methods 3(3):e45 (2016)).

Various assays may be used to determine whether the plant cell shows a change in gene expression, for example, Northern blotting or quantitative reverse transcriptase PCR (RT-PCR). Whole transgenic plants may be regenerated from the transformed cell by conventional methods. Such transgenic plants may be propagated and self-pollinated to produce homozygous lines. Such plants produce seeds containing the genes for the introduced trait and can be grown to produce plants that will produce the selected phenotype.

Modified alkaloid content, effected in accordance with the present technology, can be combined with other traits of interest, such as disease resistance, pest resistance, high yield or other traits. For example, a stable genetically engineered transformant that contains a suitable transgene that modifies alkaloid content may be employed to introgress a modified alkaloid content trait into a desirable commercially acceptable genetic background, thereby obtaining a cultivar or variety that combines a modified alkaloid level with said desirable background. For example, a genetically engineered tobacco plant with reduced nicotine may be employed to introgress the reduced nicotine trait into a tobacco cultivar with disease resistance trait, such as resistance to TMV, blank shank, or blue mold. Alternatively, cells of a modified alkaloid content plant of the present technology may be transformed with nucleic acid constructs conferring other traits of interest.

Constructs according to the present technology may be introduced into any plant cell, using a suitable technique, such as Agrobacterium-mediated transformation, particle bombardment, electroporation, and polyethylene glycol fusion, or cationic lipid-mediated transfection.

Such cells may be genetically engineered with a nucleic acid construct of the present technology without the use of a selectable or visible marker and transgenic organisms may be identified by detecting the presence of the introduced construct. The presence of a protein, polypeptide, or nucleic acid molecule in a particular cell can be measured to determine if, for example, a cell has been successfully transformed or transfected. For example, and as routine in the art, the presence of the introduced construct can be detected by PCR or other suitable methods for detecting a specific nucleic acid or polypeptide sequence. Additionally, genetically engineered cells may be identified by recognizing differences in the growth rate or a morphological feature of a transformed cell compared to the growth rate or a morphological feature of a non-transformed cell that is cultured under similar conditions. See WO 2004/076625.

The present technology also contemplates cell culture systems comprising genetically engineered cells transformed with the nucleic acid molecules described herein.

### F. Quantifying Alkaloid Content

In some embodiments of the present technology, genetically engineered plants and cells are characterized by reduced alkaloid content.

A quantitative reduction in alkaloid levels can be assayed by several methods, as for example by quantification based on gas-liquid chromatography, high performance liquid chromatography, radio-immunoassays, and enzyme-linked immunosorbent assays.

In describing a plant of the present technology, the phrase “decreased alkaloid plant” or “reduced alkaloid plant” encompasses a plant that has a decrease in alkaloid content to a level less than about 50%, about 40%, about 30%, about 25%, about 20%, about 15%, about 10%, about 9%, about 8%, about 7%, about 6%, about 5%, about 4%, about 3%, about 2% or about 1% of the alkaloid content of a control plant of the same species or variety.

### VII. Products

The methods described herein may be used for production of plants with altered alkaloid levels.

Plants of the present technology may be useful in the production of products derived from harvested portions of the plants. For example, decreased-alkaloid tobacco plants may be useful in the production of reduced-nicotine cigarettes for smoking cessation.

### VIII. Definitions

All technical terms employed in this specification are commonly used in biochemistry, molecular biology and agriculture; hence, they are understood by those skilled in the field to which the present technology belongs. Those technical terms can be found, for example in: Molecular Cloning: A Laboratory Manual 3rd ed., vol. 1-3, ed. Sambrook and Russel (Cold Spring Harbor Laboratory Press, Cold Spring Harbor, N.Y., 2001); Current Protocols In Molecular Biology, ed. Ausubel et al., (Greene Publishing Associates and Wiley-Interscience, New York, 1988) (including periodic updates); Short Protocols In Molecular Biology: A Compendium Of Methods From Current Protocols In Molecular Biology 5th ed., vol. 1-2, ed. Ausubel et al., (John Wiley & Sons, Inc., 2002); Genome Analysis: A Laboratory Manual, vol. 1-2, ed. Green et al., (Cold Spring Harbor Laboratory Press, Cold Spring Harbor, N.Y., 1997). Methodology involving plant biology techniques are described here and also are described in detail in treatises such as Methods In Plant Molecular Biology: A Laboratory Course Manual, ed. Maliga et al., (Cold Spring Harbor Laboratory Press, Cold Spring Harbor, N.Y., 1995).

An “alkaloid” is a nitrogen-containing basic compound found in plants and produced by secondary metabolism. A “pyrrolidine alkaloid” is an alkaloid containing a pyrrolidine ring as part of its molecular structure, for example, nicotine. Nicotine and related alkaloids are also referred to as pyridine alkaloids in the published literature. A “pyridine alkaloid” is an alkaloid containing a pyridine ring as part of its molecular structure, for example, nicotine. A “nicotinic alkaloid” is nicotine or an alkaloid that is structurally related to nicotine and that is synthesized from a compound produced in the nicotine biosynthesis pathway. Illustrative nicotinic alkaloids include but are not limited to nicotine, nornicotine, anatabine, anabasine, anatalline, N-methylanatabine, N-methylanabasine, myosmine, anabaseine, formylnornicotine, nicotyrine, and cotinine. Other very minor nicotinic alkaloids in tobacco leaf are reported, for example, in Hecht et al., Accounts of Chemical Research 12: 92-98 (1979); Tso, T. G., Production, Physiology and Biochemistry of Tobacco Plant. Ideals Inc., Beltsville, MO (1990).

As used herein “alkaloid content” means the total amount of alkaloids found in a plant, for example, in terms of pg/g dry weight (DW) or ng/mg fresh weight (FW). “Nicotine content” means the total amount of nicotine found in a plant, for example, in terms of mg/g DW or FW.

A “chimeric nucleic acid” comprises a coding sequence or fragment thereof linked to a nucleotide sequence that is different from the nucleotide sequence with which it is associated in cells in which the coding sequence occurs naturally.

The terms “encoding” and “coding” refer to the process by which a gene, through the mechanisms of transcription and translation, provides information to a cell from which a series of amino acids can be assembled into a specific amino acid sequence to produce an active enzyme. Because of the degeneracy of the genetic code, certain base changes in DNA sequence do not change the amino acid sequence of a protein.

“Endogenous nucleic acid” or “endogenous sequence” is “native” to, i.e., indigenous to, the plant or organism that is to be genetically engineered. It refers to a nucleic acid, gene, polynucleotide, DNA, RNA, or mRNA, molecule that is present in the genome of a plant or organism that is to be genetically engineered.

“Exogenous nucleic acid” refers to a nucleic acid, DNA or RNA, which has been introduced into a cell (or the cell's ancestor) through the efforts of humans. Such exogenous nucleic acid may be a copy of a sequence which is naturally found in the cell into which it was introduced, or fragments thereof.

As used herein, “expression” denotes the production of an RNA product through transcription of a gene or the production of the polypeptide product encoded by a nucleotide sequence. “Overexpression” or “up-regulation” is used to indicate that expression of a particular gene sequence or variant thereof, in a cell or plant, including all progeny plants derived thereof, has been increased by genetic engineering, relative to a control cell or plant.

“Genetic engineering” encompasses any methodology for introducing a nucleic acid or specific mutation into a host organism. For example, a plant is genetically engineered when it is transformed with a polynucleotide sequence that suppresses expression of a gene, such that expression of a target gene is reduced compared to a control plant. A plant is genetically engineered when a polynucleotide sequence is introduced that results in the expression of a novel gene in the plant, or an increase in the level of a gene product that is naturally found in the plants. In the present context, “genetically engineered” includes transgenic plants and plant cells, as well as plants and plant cells produced by means of targeted mutagenesis effected, for example, through the use of chimeric RNA/DNA oligonucleotides, as described by Beetham et al., Proc. Natl. Acad. Sci. U.S.A. 96: 8774-8778 (1999) and Zhu et al., Proc. Natl. Acad. Sci. U.S.A. 96: 8768-8773 (1999), or so-called “recombinagenic olionucleobases,” as described in International patent publication WO 2003/013226. Likewise, a genetically engineered plant or plant cell may be produced by the introduction of a modified virus, which, in turn, causes a genetic modification in the host, with results similar to those produced in a transgenic plant. See, e.g., U.S. Pat. No. 4,407,956. Additionally, a genetically engineered plant or plant cell may be the product of any native approach (i.e., involving no foreign nucleotide sequences), implemented by introducing only nucleic acid sequences derived from the host plant species or from a sexually compatible plant species. See, e.g., U.S. Patent Application No. 2004/0107455.

“Heterologous nucleic acid” refers to a nucleic acid, DNA, or RNA, which has been introduced into a cell (or the cell's ancestor), and which is not a copy of a sequence naturally found in the cell into which it is introduced. Such heterologous nucleic acid may comprise segments that are a copy of a sequence that is naturally found in the cell into which it has been introduced, or fragments thereof.

“Homozygous” and “homozygosity” may be used interchangeably herein. A plant is homozygous when the alleles of a gene residing on a homologous chromosome pair are identical. All gametes arising from this plant are identical at that gene locus and such plants do not segregate on selfing. Thus, non-segregating genotypes constitute homozygous populations.

By “isolated nucleic acid molecule” is intended a nucleic acid molecule, DNA, or RNA, which has been removed from its native environment. For example, recombinant DNA molecules contained in a DNA construct are considered isolated for the purposes of the present technology. Further examples of isolated DNA molecules include recombinant DNA molecules maintained in heterologous host cells or DNA molecules that are purified, partially or substantially, in solution. Isolated RNA molecules include in vitro RNA transcripts of the DNA molecules of the present technology. Isolated nucleic acid molecules, according to the present technology, further include such molecules produced synthetically.

“Plant” is a term that encompasses whole plants, plant organs (e.g., leaves, stems, roots, etc.), seeds, differentiated or undifferentiated plant cells, and progeny of the same. Plant material includes without limitation seeds, suspension cultures, embryos, meristematic regions, callus tissues, leaves, roots, shoots, stems, fruit, gametophytes, sporophytes, pollen, and microspores.

“Plant cell culture” means cultures of plant units such as, for example, protoplasts, cell culture cells, cells in plant tissues, pollen, pollen tubes, ovules, embryo sacs, zygotes, and embryos at various stages of development. In some embodiments of the present technology, a transgenic tissue culture or transgenic plant cell culture is provided, wherein the transgenic tissue or cell culture comprises a nucleic acid molecule of the present technology.

“Decreased alkaloid plant” or “reduced alkaloid plant” encompasses a genetically engineered plant that has a decrease in alkaloid content to a level less than 50%, and preferably less than 10%, 5%, or 1% of the alkaloid content of a control plant of the same species or variety.

“Increased alkaloid plant” encompasses a genetically engineered plant that has an increase in alkaloid content greater than 10%, and preferably greater than 50%, 100%, or 200% of the alkaloid content of a control plant of the same species or variety.

“Promoter” connotes a region of DNA upstream from the start of transcription that is involved in recognition and binding of RNA polymerase and other proteins to initiate transcription. A “constitutive promoter” is one that is active throughout the life of the plant and under most environmental conditions. Tissue-specific, tissue-preferred, cell type-specific, and inducible promoters constitute the class of “non-constitutive promoters.” “Operably linked” refers to a functional linkage between a promoter and a second sequence, where the promoter sequence initiates and mediates transcription of the DNA sequence corresponding to the second sequence. In general, “operably linked” means that the nucleic acid sequences being linked are contiguous.

As used herein, the term “recognition sequence” refers to a DNA sequence that is bound and cleaved by an endonuclease. In the case of a meganuclease, a recognition sequence may comprise, for example, a pair of inverted, 9 basepair “half sites” which are separated by four basepairs. In some embodiments, in the case of a single-chain meganuclease, the N-terminal domain of the protein contacts a first half-site and the C-terminal domain of the protein contacts a second half-site. Cleavage by a meganuclease produces four basepair 3′ “overhangs.” “Overhangs,” or “sticky ends” are short, single-stranded DNA segments that can be produced by endonuclease cleavage of a double-stranded DNA sequence. In the case of meganucleases and single-chain meganucleases derived from I-CreI, the overhang comprises bases 10-13 of the 22 basepair recognition sequence. [0035]

As used herein, the term “target site” or “target sequence” refers to a region of the chromosomal DNA of a cell comprising a recognition sequence for a meganuclease.

“Sequence identity” or “identity” in the context of two polynucleotide (nucleic acid) or polypeptide sequences includes reference to the residues in the two sequences that are the same when aligned for maximum correspondence over a specified region. When percentage of sequence identity is used in reference to proteins it is recognized that residue positions which are not identical often differ by conservative amino acid substitutions, where amino acid residues are substituted for other amino acid residues with similar chemical properties, such as charge and hydrophobicity, and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, for example, according to the algorithm of Meyers & Miller, Computer Applic. Biol. Sci. 4: 11-17 (1988), as implemented in the program PC/GENE (Intelligenetics, Mountain View, California, USA).

Use in this description of a percentage of sequence identity denotes a value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The terms “suppression” or “down-regulation” are used synonymously to indicate that expression of a particular gene sequence variant thereof, in a cell or plant, including all progeny plants derived thereof, has been reduced by genetic engineering, relative to a control cell or plant (e.g., “MPO down-regulation”).

As used herein, a “synergistic effect” refers to a greater-than-additive effect (e.g., effect on alkaloid content) that is produced by combined modulation of at least two nicotine biosynthesis genes or families of nicotine biosynthesis genes (e.g., the effect produced by a combined suppression of at least two enzymes of the nicotine biosynthesis pathway, such as at least one MPO (e.g., MPO1.1, MPO1.2, MPO2.1, and/or MPO2.2), and at least one BBL (e.g., BBLa, BBLb, and/or BBLc), and which exceeds that which would otherwise result from modulation of the individual nicotine biosynthesis gene (e.g., the effect produced by the suppression of one or more of MPO1.1, MPO1.2, MPO2.1, and/or MPO2.2 alone, or one or more of BBLa, BBLb, and/or BBLc alone).

“Tobacco” or “tobacco plant” refers to any species in the Nicotiana genus that produces nicotinic alkaloids, including but not limited to the following: Nicotiana acaulis, Nicotiana acuminata, Nicotiana acuminata var. multzjlora, Nicotiana africana, Nicotiana alata, Nicotiana amplexicaulis, Nicotiana arentsii, Nicotiana attenuata, Nicotiana benavidesii, Nicotiana benthamiana, Nicotiana bigelovii, Nicotiana bonariensis, Nicotiana cavicola, Nicotiana clevelandii, Nicotiana cordifolia, Nicotiana corymbosa, Nicotiana debneyi, Nicotiana excelsior, Nicotiana forgetiana, Nicotiana fragrans, Nicotiana glauca, Nicotiana glutinosa, Nicotiana goodspeedii, Nicotiana gossei, Nicotiana hybrid, Nicotiana ingulba, Nicotiana kawakamii, Nicotiana knightiana, Nicotiana langsdorfi, Nicotiana linearis, Nicotiana longiflora, Nicotiana maritima, Nicotiana megalosiphon, Nicotiana miersii, Nicotiana noctiflora, Nicotiana nudicaulis, Nicotiana obtusifolia, Nicotiana occidentalis, Nicotiana occidentalis subsp. hesperis, Nicotiana otophora, Nicotiana paniculata, Nicotiana pauczjlora, Nicotiana petunioides, Nicotiana plumbaginfolia, Nicotiana quadrivalvis, Nicotiana raimondii, Nicotiana repanda, Nicotiana rosulata, Nicotiana rosulata subsp. ingulba, Nicotiana rotundifolia, Nicotiana rustica, Nicotiana setchellii, Nicotiana simulans, Nicotiana solanifolia, Nicotiana spegauinii, Nicotiana stocktonii, Nicotiana suaveolens, Nicotiana sylvestris, Nicotiana tabacum, Nicotiana thyrsiflora, Nicotiana tomentosa, Nicotiana tomentosifomis, Nicotiana trigonophylla, Nicotiana umbratica, Nicotiana undulata, Nicotiana velutina, Nicotiana wigandioides, and interspecific hybrids of the above.

“Tobacco product” refers to a product comprising material produced by a Nicotiana plant, including for example, cut tobacco, shredded tobacco, nicotine gum and patches for smoking cessation, cigarette tobacco including expanded (puffed) and reconstituted tobacco, cigar tobacco, pipe tobacco, cigarettes, cigars, and all forms of smokeless tobacco such as chewing tobacco, snuff, snus, and lozenges.

A “variant” is a nucleotide or amino acid sequence that deviates from the standard, or given, nucleotide or amino acid sequence of a particular gene or polypeptide. The terms “isoform,” “isotype,” and “analog” also refer to “variant” forms of a nucleotide or an amino acid sequence. An amino acid sequence that is altered by the addition, removal, or substitution of one or more amino acids, or a change in nucleotide sequence, may be considered a variant sequence. A polypeptide variant may have “conservative” changes, wherein a substituted amino acid has similar structural or chemical properties, e.g., replacement of leucine with isoleucine. A polypeptide variant may have “nonconservative” changes, e.g., replacement of a glycine with a tryptophan. Analogous minor variations may also include amino acid deletions or insertions, or both. Guidance in determining which amino acid residues may be substituted, inserted, or deleted may be found using computer programs well known in the art such as Vector NTI Suite (InforMax, MD) software. Variant may also refer to a “shuffled gene” such as those described in Maxygen-assigned patents (see, e.g., U.S. Pat. No. 6,602,986).

A “wild type” or “native” nucleic acid, nucleotide sequence, polypeptide or amino acid sequence refers to a naturally occurring or endogenous nucleic acid, nucleotide sequence, polypeptide or amino acid sequence. Thus, for example, a “wild type nucleic acid” is a nucleic acid that is naturally occurring in or endogenous to the organism. A “homologous” nucleic acid sequence is a nucleotide sequence naturally associated with a host cell into which it is introduced. A “wild type” strain or line of tobacco, as used herein, refers to a tobacco strain or line that does not comprise the MPO or BBLabc mutations.

In some embodiments, as described herein, the plants of the present technology comprise BBLabc mutations that are homozygous for the three major bbl mutations (e.g., bbla bbla, bblb bblb, bblc bblc). As used herein, “BBLa” refers to a nucleic acid sequence or fragment thereof having at least 85% identity to a nucleic acid that encodes the polypeptide set forth in GenBank Accession No. AB604219. The polypeptide sequence of the exemplary BBLa is set forth in SEQ ID NO: 16. As used herein, “BBLb” refers to a nucleic acid sequence or fragment thereof having at least 85% identity to a nucleic acid that encodes the polypeptide set forth in GenBank Accession No. AM851017. The polypeptide sequence of the exemplary BBLb is set forth in SEQ ID NO: 17. As used herein, “BBLc” refers to a nucleic acid sequence or fragment thereof having at least 85% identity to a nucleic acid that encodes the polypeptide set forth in GenBank Accession No. AB604220. The polypeptide sequence of the exemplary BBLc is set forth in SEQ ID NO: 18.

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

### EXAMPLES

The following examples are provided by way of illustration only and not by way of limitation. Those of skill in the art will readily recognize a variety of non-critical parameters that could be changed or modified to yield essentially the same or similar results. The examples should in no way be construed as limiting the scope of the present technology, as defined by the appended claims.

### Example 1: Generation of Low Nicotine Tobacco Plants Through the RNAi-Directed Suppression of MPO Gene Expression in a Triple Homozygous Mutant BBLa, BBLb, BBLc Background

This example demonstrates the efficacy of combining the suppression of MPO gene function with mutations in the three major BBL genes (BBLa, BBLb, and BBLc) to reduce nicotine content and prevent anatabine accumulation in tobacco plants.

### Materials and Methods

Two RNAi constructs (MPO6 and MPO10) were generated based upon conserved 155 bp and 242 bp sequences found in exons 6 (SEQ ID NO: 13) and 10 (SEQ ID NO: 14) of MPO1.1, respectively. The sense and antisense cassettes were cloned on either side of a soybean FAD3 intron using vector pKYLX80i, then the entire ‘sense-FAD3 intron-antisense’ fragment was inserted into plant binary expression vector pKLX71 as previously described (Siminszky et al., 2005). The resulting plant expression vectors were subsequently introduced into Agrobacterium and tobacco leaf discs were transformed according to the protocol of Horsch et al. (1985) using nptI as the selectable marker. Tobacco variety K326, and a near isogenic derivative of K326 that possesses EMS-induced knockout mutations in the three major BBL genes (BBLa, BBLb, BBLc; “triple mutant bbl line K326 222”), designated “K326 222” (Lewis et al., 2020), were used in the transformation experiments. All T₀ plants were grown in a greenhouse until flowering, at which point two leaves from the middle of each plant were collected, dried, ground, and analyzed for alkaloid composition as previously described (Lewis et al., 2015). Analyses of variance were performed for measured characteristics using PROC MIXED of SAS 9.2 (SAS Institute, Cary, NC).

### Results

Twenty-seven primary transformants (T₀ generation) were regenerated in a wild type K326 background and 28 T₀ transformants were obtained using the triple mutant bbl line K326 222. Primary transformants exhibited a continuum for anatabine accumulation as a percent of the total alkaloid pool. Previous evaluations of non-genetically altered tobacco lines indicated that anatabine does not accumulate to levels greater than 8% of the total alkaloid pool (Lewis et al., 2020). For the sake of efficient presentation and discussion of alkaloid data for primary transformants, plants with measured anatabine being greater than 10% of the total alkaloid pool were considered to carry a functioning RNAi transgene insertion, while plants accumulating anatabine at levels less than 10% of the total alkaloid pool were considered to carry a non-functioning RNAi transgene insertion.

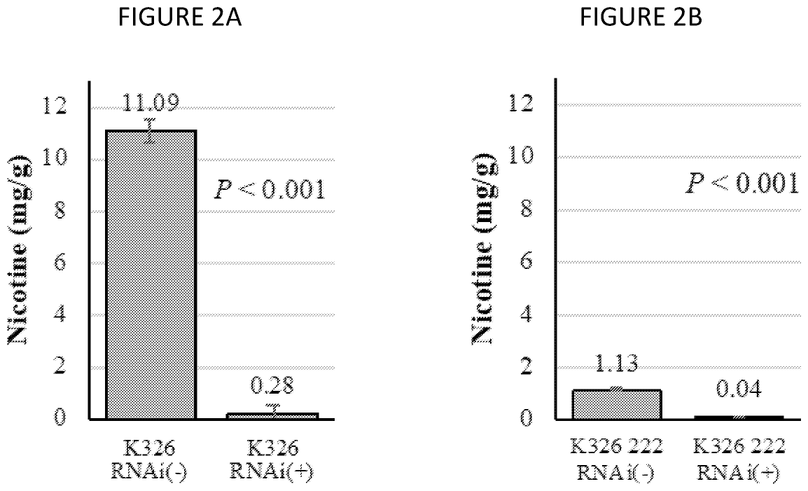

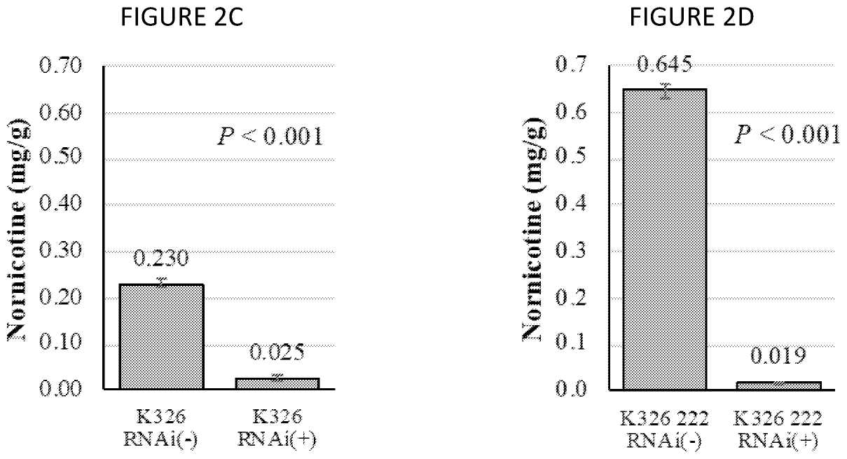

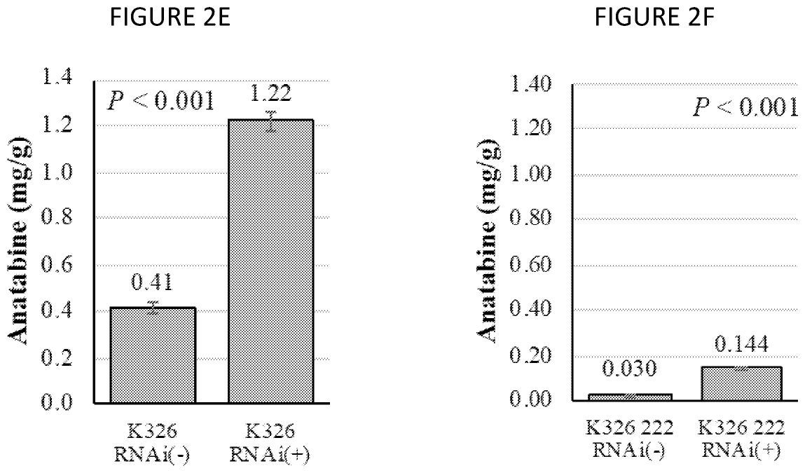

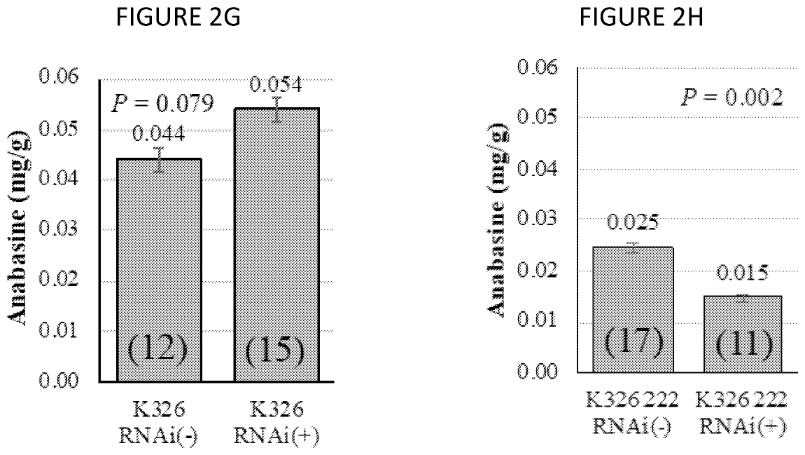

As shown in FIGS. 2A and 2C, plants categorized as carrying a functioning RNAi transgene insertion showed a 97.5% reduction in nicotine, and an 89% reduction in nornicotine in the wild type (WT) K326 background. Further reductions in nicotine (99.6%) were realized when a functional anti-MPO RNAi construct was combined with the triple mutant bbl technology (FIG. 2B). In contrast to the nicotine and nornicotine observations, in WT K326, anatabine levels were 3-fold higher in the plants presumed to be expressing an active RNAi construct (FIG. 2E). Although the ratio between anatabine and nicotine was similar in the K326 222 RNAi(−) versus RNAi(+) plants, the gross accumulation of anatabine in both cases was lower in the K326 222 plants than their WT K326 counterparts (FIGS. 2E and 2F).

Accordingly, these results demonstrate that combining mpo mutations with bbl mutations in tobacco plants is useful in methods for producing tobacco plants characterized by significantly reduced nicotine levels and lacking the accumulation of anatabine that typically results in tobacco plants having mpo mutations, as compared to wild-type control tobacco plants and/or plants in which the mpo mutations are present but which lack the bbl mutations.

### Example 2: Generation of Low Nicotine Tobacco Plants Through Meganuclease-Mediated Knockout of MPO Genes in a Triple Homozygous Mutant BBLa, BBLb, BBLc Background

This example demonstrates the efficacy of combining the suppression of MPO gene function with mutations in the three major BBL genes (BBLa, BBLb, and BBLc) to reduce nicotine content and prevent anatabine accumulation in tobacco plants.

### Materials and Methods

A proprietary ARCUS® meganuclease (or homing endonuclease) genome editing construct (Precision Biosciences, Durham, NC) was strategically designed to recognize the 22mer sequence 5′-ATCTATGGGTTACACAATATGC-3′ (SEQ ID NO: 15) that is found in exon 10 of MPO1.1, MPO1.2 and MPO2.2. Due to a single polymorphism located at position 15 of the 22mer sequence in MPO2.1, this MPO isoform only matches 21 of the 22 sequences of the MPO-specific ARCUS® recognition site. The ARCUS® construct was introduced into tobacco varieties K326, TN90, and their triple mutant bbl counterparts K326 222 and TN90 222 (Lewis et al., 2020) using standard Agrobacterium-based transformation protocols (Horsch et al., 1985).

T₀ generation transgenic plants that were identified as possessing meganuclease-induced mutations in MPO genes (as determined by DNA sequence analysis) were self-pollinated and genotyped to identify T₁ individuals that contained knockout mutations in both alleles in each MPO gene where a mutation was observed in at least one allele in the T₀ parent. Selected T₁ generation plants were grown in a greenhouse until the first sign of floral bud development, at which time the plants were topped. Suckers were removed by hand for a 10-day period, after which a similar sized upper stalk position leaf was harvested, dried, and ground for alkaloid analysis as previously described (Lewis et al. 2015). Statistical analysis was conducted using SAS 9.2 software (SAS Institute, Cary, NC).

### Results

The ARCUS® meganuclease genome editing technology of Precision Biosciences was used to introduce mutations into MPO genes of tobacco lines K326, K326 222, TN90, and TN90 222. Genomic DNA isolated from young leaf tissue of each T₀ plant was amplified using PCR using primers specific for each of the four MPO gene isoforms. DNA sequence analyses of the PCR products were conducted to determine whether an ARCUS-induced mutation was introduced at that locus. In most cases, examination of the resulting sequence chromatograms enabled us to categorize each MPO gene as falling into one of the following categories: (1) homozygous WT at both alleles; (2) heterozygous for a meganuclease-induced mutation at one allele and WT at the other; (3) homozygous for the same meganuclease-induced mutation at both alleles; or (4) heterozygous for one specific meganuclease-induced mutation at one allele and possessing a different meganuclease-induced mutation at the other allele. Because previous studies suggested that MPO1.1 and MPO1.2 are the predominant, if not sole, sources of MPO enzyme activity in tobacco plants (Naconsie et al., 2014), priority was given to T₀ individuals where at least one knockout mutant allele in both MPO1.1 and MPO1.2 could be identified.

T₀ individuals with promising MPO mutations were self-pollinated and the resulting T₁ progeny were genotyped by DNA sequence analysis to identify T₁ plants containing knockout mutations in each allele of a given MPO gene. The lines selected for further analysis are listed in Table 1, and the specific endonuclease-induced mutations found in each line are shown in Table 2.

TABLE 1
Meganuclease-induced mutations in tobacco MPO genes.

| Line | MPO1.1 | MPO2.1 | MPO1.2 | MPO2.2 |
|---|---|---|---|---|
| K11 (K326 WT | 7 bp del | WT | 1 bp ins/8 bp | 222 bp del |
| background) | | | del | |
| K23 (K326 WT | 1 bp ins | WT | 49 bp del + 3 | 22 bp del + 5 bp ins |
| background) | | | bp ins | |
| KB19 (K326 222 | 4 bp del/1 | 1 bp del | 7 bp del | 25 bp ins + 4 bp del/ |
| background) | bp ins | | | 48 bp del + 1 bp ins |
| KB20 (K326 222 | 25 bp del | WT | 11 bp del/87 | WT |
| background) | | | bp del | |
| T23 (TN90 WT | 1 bp ins | WT | 190 bp del + 4 | 1 bp ins/27 bp del + |
| background) | | | bp ins | 16 bp ins |
| TB7 (TN90 222 | 1 bp del/14 | WT | 19 bp del + 3 | 1 bp ins |
| background) | bp del | | bp ins | |

Some lines are segregating for mutations at select MPO loci.
The alternative allele mutations at these loci are separated by a forward slash (/).
Del = deletion; Ins = insertion.
Lines labeled 222 possess EMS-induced knockout mutations in the genes BBLa, BBLb, and BBLc as described in Lewis et al., 2020.

TABLE 2
Meganuclease-induced mutations found in the MPO genes of the lines developed (Table 1). Sequence shown are in exon 10 of the respective MPO genes. The underlined 22 bp region represents the ARCUS®
meganuclease recognition site. Short deletions are representedby dashes; long deletions are shown as dotted lines. Insertion events are shown in bold, italicized type.

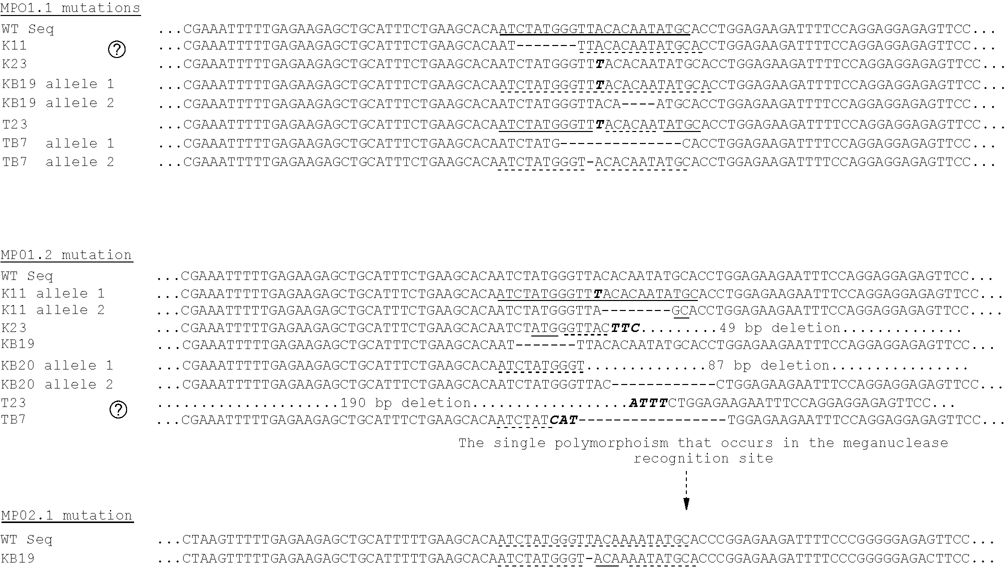

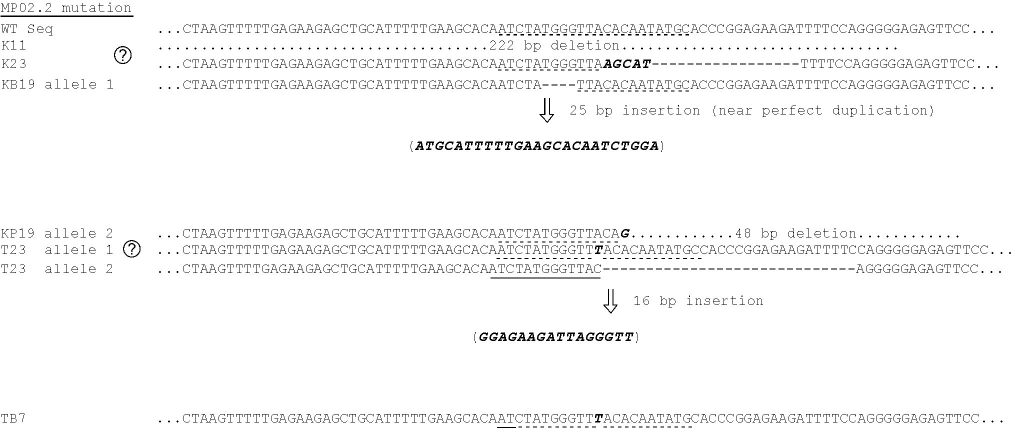

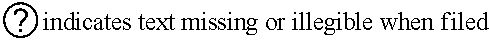

In the K326 WT background, two lines (K11 and K23) were produced that carried mutant mpo1.1, mpo1.2 and mpo2.2 alleles. Two independent lines were also obtained in K326 222. In KB19, all four MPO genes possess knockout mutations. The fact that a mutation in MPO2.1 in this plant was recovered was somewhat surprising given that there is a polymorphism in the 22mer ARCUS® meganuclease recognition site in this gene (SEQ ID NO: 7; Table 2). The other mutant line in K326 222, designated KB20, is mutant at both MPO1 loci, and wild type at the two MPO2 genes. The ARCUS® meganuclease-mediated targeted mutagenesis effort in the burley market type TN90 yielded two lines, one in TN90 WT (T23) and the other in TN90 222 (TB7). Both lines carry knockout mpo1.1, mpo1.2 and mpo2.2 alleles.

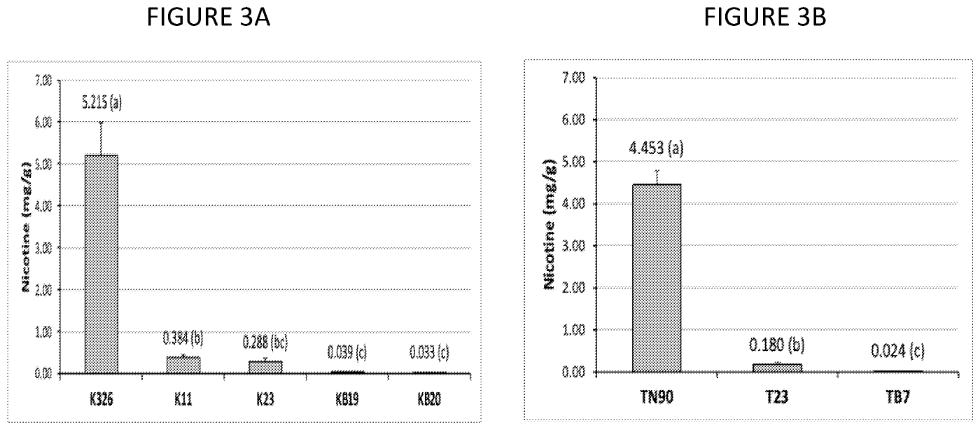

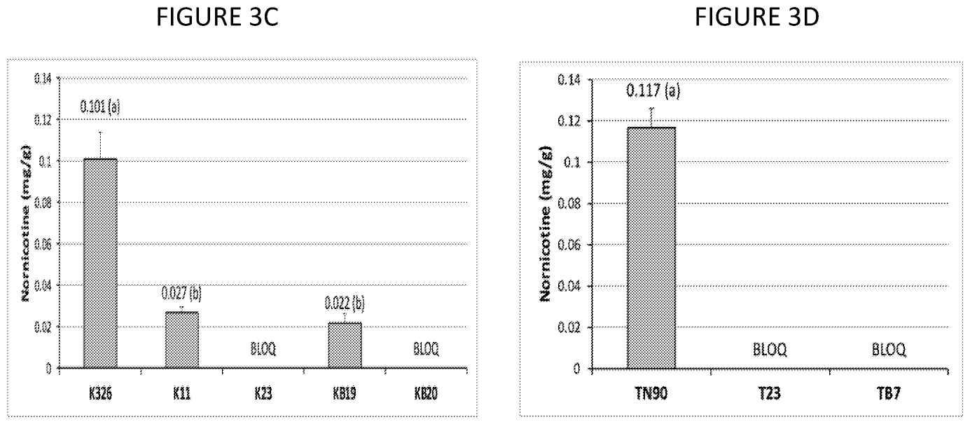

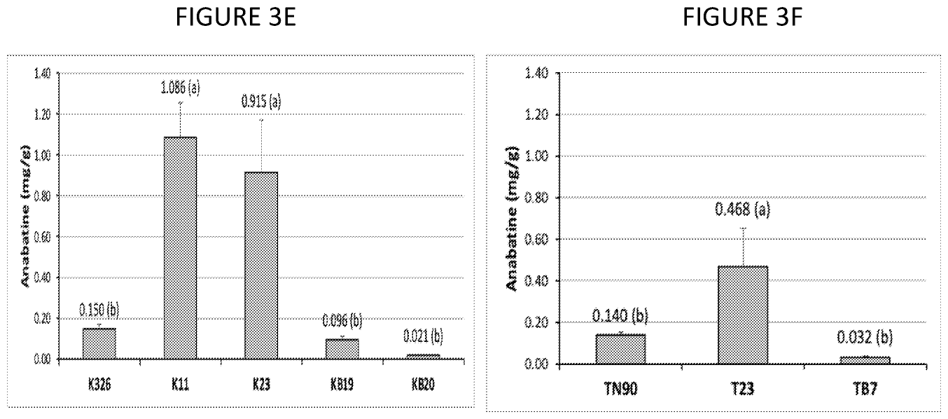

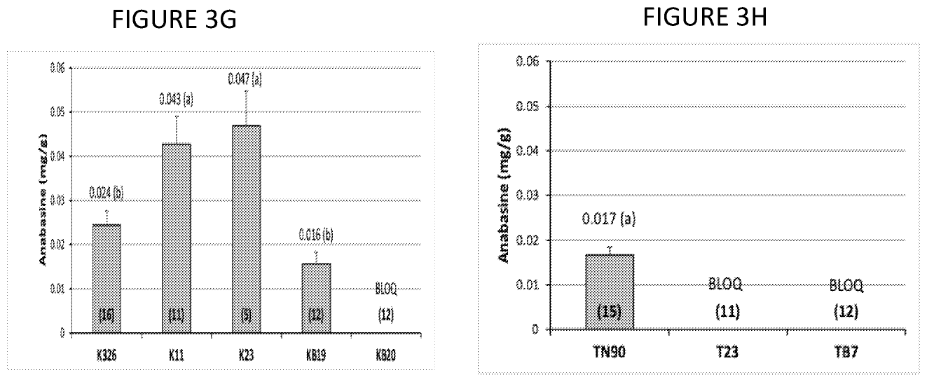

To determine the effect of the MPO mutations on alkaloid accumulation, T₁ generation plants whose allelic complement of mutations had been verified by DNA sequence analysis were grown to maturity in a greenhouse. Upper position leaves of similar size from plants that had been topped for ten days were harvested and analyzed for the four major alkaloids nicotine, nornicotine, anatabine, and anabasine. Every line was represented by more than 10 plants, with the exception of K23 which had 5 individuals. The results are shown in FIGS. 3A-3H. Mutations in MPO genes resulted in nicotine reductions between 93-96% in the WT K326 and TN90 backgrounds, and dropped to greater than 99% when the MPO mutations were combined with the three BBL mutations (FIGS. 3A-3B). Comparisons between KB19 and KB20 were of particular interest since the former possesses mutations in all four MPO genes, whereas the latter was only mutated at the two MPO1 loci. None of the four alkaloids measured were lower in KB19 compared with KB20, suggesting that MPO2.1 and MPO2.2 may not play a significant role in alkaloid biosynthesis.

The effects of MPO mutagenesis on the anatabine content was particularly noteworthy. Similar to the RNAi results presented in Example 1 (FIGS. 2E and 2F) and predictions based on the literature (Shoji and Hashimoto, 2008), knocking out MPO gene function in the K326 and TN90 WT backgrounds resulted in a 3- to 7-fold higher accumulation of anatabine than their respective controls (FIGS. 3E-3F). However, when MPO mutations were introduced into the triple bbl mutant K326 222 and TN90 222 backgrounds, the anatabine content dropped to a level that was numerically lower than their WT counterparts (though not to an extent deemed statistically significant) (FIGS. 3E-3F).

Nornicotine levels were significantly lower in all of the lines possessing MPO mutations compared to their WT controls (FIGS. 3C-3D). Interestingly, the anabasine content in the K11 and K23 lines with only mpo mutations was nearly double that observed in K326 WT, but this phenomenon was not seen in the TN90 background (FIGS. 3G-3H). Regardless, in each of the K326 222 and TN90 222 backgrounds, the amount of anabasine observed was either equivalent to the WT control (KB19), or below the level of quantification (KB20 and TB7) (FIGS. 3G-3H).

Accordingly, these results demonstrate that combining mpo mutations with bbl mutations in tobacco plants is useful in methods for producing tobacco plants characterized by significantly reduced nicotine levels and lacking the accumulation of anatabine that typically results in tobacco plants having mpo mutations, as compared to wild-type control tobacco plants and/or plants in which the mpo mutations are present but which lack the bbl mutations.

### Summary of Results

Using both RNAi-mediated knockdown of MPO gene expression and genome editing-based knockout of select MPO genes, the experimental examples provided herein demonstrate that dramatic reductions in the nicotine content of the leaf can be achieved in whole tobacco plants. One characteristic of the low nicotine tobaccos developed by MPO disruption is that these plants display an accompanying increase in anatabine to levels far above that which is found in normal tobacco plants. Because the pharmacological effects of anatabine and/or its derivatives have been minimally investigated in comparison to nicotine, the high anatabine trait could prove detrimental to consumer acceptance and/or regulatory approval by the FDA. Here, the inventors demonstrate that combining mpo mutations with bbl mutations confers two marked advantages: (1) the nicotine levels in mpo+bbl mutant plants is significantly lower than that which is observed by the mutant mpo and bbl technologies individually; and (2) the potentially problematic elevated anatabine trait associated with MPO inhibition becomes ameliorated when combined with three bbl mutations.

### Example 3: Generation of Low Nicotine Tobacco Plants Through Meganuclease-Mediated Knockout of MPO Genes in a Triple Homozygous Mutant BBLa, BBLb, BBLc Background—Field Studies

This example demonstrates the efficacy of combining the suppression of MPO gene function with mutations in the three major BBL genes (BBLa, BBLb, and BBLc) to reduce nicotine content and prevent anatabine accumulation in tobacco plants grown in a field.

### Materials and Methods

Lines selected for field evaluation were initially seeded onto float trays prior to transplanting to the field. The K326-based genotypes were grown in a separate plot from the TN90-based genotypes to accommodate the differences in fertilization regimes optimal for flue-cured versus burley tobacco market types. In both experiments, the plants were grown using a randomized complete block design comprised of 30 replications with each individual plant defined as an experimental unit. Flue-cured lines K326 WT, K326 bbla/b/c (also referred to herein as “K326 222”), K11, KB19 and KB20, were fertilized with 95.3 kg N/ha, and burley lines TN90 LC, TN90 bbla/b/c (also referred to herein as “TN90 222”), T23 and TB7, were provided 320 kg N/ha. Plants were topped prior to flowering and treated with FluPro to prevent sucker formation, according to the manufacturer's instruction (Chemtura Agro Solutions, USA).

At maturity, plants were cut at the base and weighed to determine above-ground (“aerial”) biomass. The fourth leaf from the top of each plant was excised, and after removal of the mid-rib, the lamina was placed in paper bags and dried to completeness in a drying over at 65° C. After grinding the dried lamina to a fine powder, each sample was analyzed for alkaloid content by the NCSU Tobacco Analytical Lab as previously described (Lewis et al., 2015). Statistical analysis was performed using SAS 9.2 software (SAS Institute, Cary, NC).

### Results

To determine the effect of knockout mutations in MPO genes in a field environment, both alone and in the presence of mutations in the three major BBL genes, all of the lines described in Table 1, with the exception of K23 due to a lack of sufficient seed, were grown at the Central Crops Research Station in Clayton, North Carolina. As an additional control, lines K326 bbla/b/c (also referred to herein as “K326 222”) and TN90 bbla/b/c (also referred to herein as “TN90 222”), which only contain mutations in the three major BBL genes were included in this study. Because burley tobaccos require a much high level of N fertilization in comparison to flue-cured varieties, the K326-based and TN90-based genotypes were grown as separate experiments at different locations in the field. Although 30 plants per genotype were transplanted to the field (in a randomized complete block design), a small number of plants in the K326 background died during the course of the experiment. Therefore, the total number of plants per genotype that were evaluated ranged from 25-30. Due to the combination of infection by the pathogen brown spot, and a high incidence of “fire up” (a premature senescence phenomenon common to burley tobaccos), a greater number of plants were lost in the TN90-based experiment; the total number of plants per genotype ranged from 17-22.

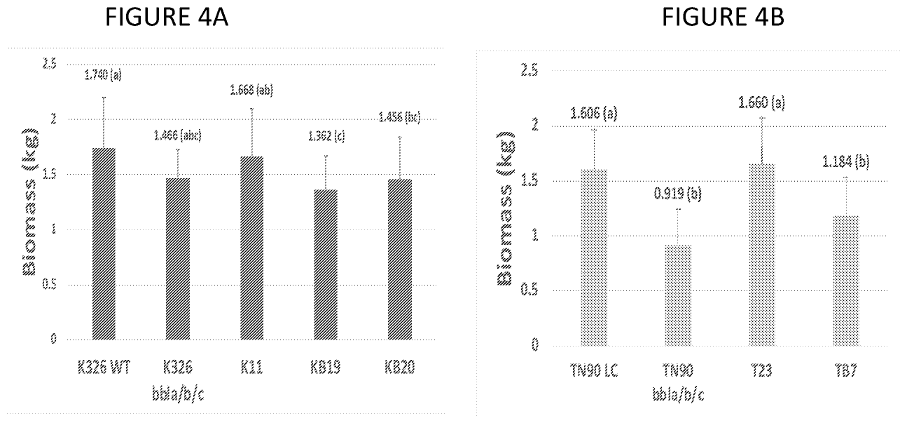

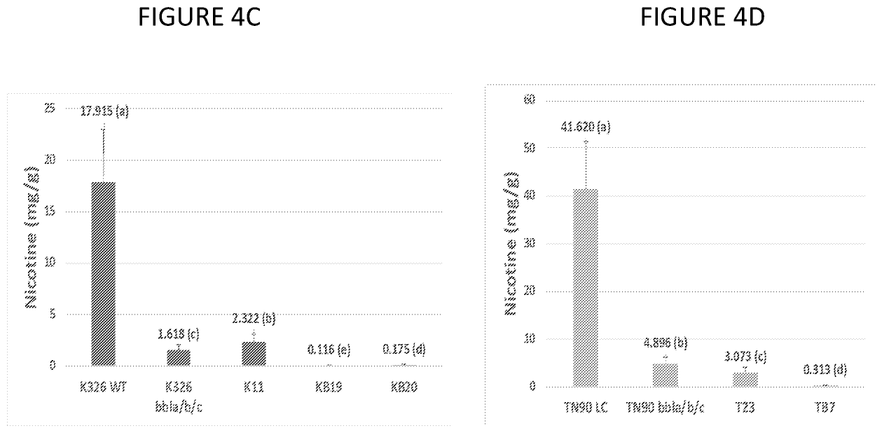

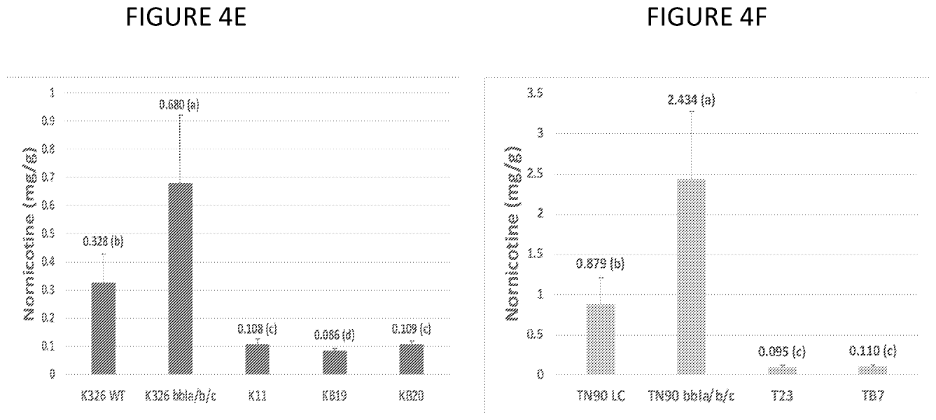

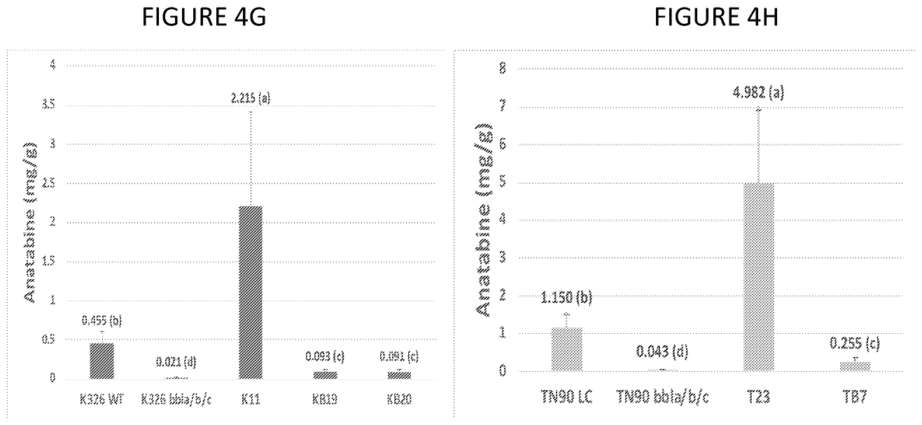

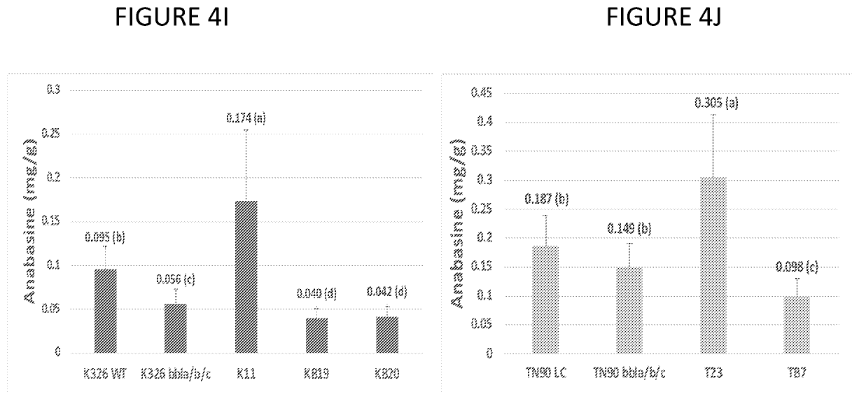

At maturity, all plants were cut at the base and weighed to determine aerial biomass. These results are shown in FIGS. 4A and 4B. No significant differences in average plant biomass were observed in plants possessing only mpo mutations (K11 and T23) in comparison to their parental controls (K326 WT and TN90 LC, respectively). In contrast, a decrease in biomass was observed in all lines possessing bbl mutations. In the K326 experiment, the decrease was modest, with a 16% reduction observed in lines K326 bbla/b/c and KB20, and marginally greater decrease of 22% observed in KB19. In the TN90 experiment, the effect on biomass attributable to the bbl mutations was more severe: a 43% reduction in TN90 bbla/b/c and a 27% reduction in TB7.

An alkaloid analysis was conducted on the fourth leaf from that top of each plant at maturity. The nicotine results are shown in FIGS. 4C and 4D. Nicotine levels were reduced between 88% and 93% in lines possessing only bbl (K326 bbla/b/c and TN90 bbla/b/c) or mpo (K11 and T23) mutations. More dramatic reductions were observed when mpo and bbl mutations were combined within the same plant. Lines KB19, KB20, and TB7 all demonstrated reductions in nicotine of 99% or greater in comparison to their respective controls.

FIGS. 4E and 4F show the nornicotine content of each line. A more than 2-fold increase in nornicotine was observed in lines possessing only bbl mutations (K326 bbla/b/c and TN90 bbla/b/c). In contrast, 3- to 8-fold decreases in nornicotine were observed in all lines possessing mpo mutations.

Average anatabine content is shown in FIGS. 4G and 4H. Consistent with the greenhouse-based experiment, lines containing only mpo mutations (K11 and T23) displayed much higher levels of anatabine (over 4-fold greater) than their respective parental controls. However, lines in which mpo mutations have been combined with bbl mutations, an approximate 5-fold decrease is observed in anatabine accumulation.

The anabasine content of each line is shown in FIGS. 4I and 4J. Lines possessing only mpo mutations (K11 and T23) showed 1.6- to 1.8-fold higher levels of anabasine, while lines with both mpo and bbl mutations (KB19, KB20, and TB7) accumulated approximately 2-fold less anabasine than the controls.

### Summary

Collectively, the results from the field study provide support for the advantages of combining the triple mutant bbl (bbla/b/c) trait with the mutant mpo technology as a strategy for producing tobaccos that accumulate ultra-low levels of not only nicotine, but also of all four of the major tobacco alkaloids (nicotine, nornicotine, anatabine and anabasine). Although knocking out BBL and MPO gene function alone mediated nicotine reductions in the range of 88-93%, each of the individual technologies alone suffered the drawback of causing other alkaloids to become elevated beyond that which was observed in the parental cultivar: mutations in BBL genes alone led to a greater than 2-fold increase in nornicotine, and mutations in MPO genes alone were associated with a greater than 4-fold increase in anatabine and a 1.6- to 1.8-fold increase in anabasine. Lines with mutations in both BBL and MPO genes, however, not only displayed a dramatically reduced nicotine content phenotype (99% or greater), but they were also significantly lower in all of the alkaloid species measured.

Accordingly, these results demonstrate that combining MPO mutations with BBL mutations in tobacco plants is useful in methods for producing tobacco plants characterized by significantly reduced nicotine levels and lacking the accumulation of nornicotine, anatabine and anabasine, as compared to wild-type control tobacco plants and/or plants in which the MPO or BBL mutations alone are present.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All publicly available documents referenced or cited to herein, such as patents, patent applications, provisional applications, and publications, including GenBank Accession Numbers, are incorporated by reference in their entirety, including all figures and tables, to the extent they are not inconsistent with the explicit teachings of this specification.

Other embodiments are set forth within the following claims.

### REFERENCES

Benowitz, N. L., Hall, S. M., Stewart, S., Wilson, W., Dempsey, D., and Jacob, P. 2007. Nicotine and carcinogen exposure with smoking of progressively reduced nicotine content cigarette. Cancer Epidemiol. Biomarkers Prev. 16: 2479-2485.
Benowitz, N. L. and Henningfield, J. E. 1994. Establishing a nicotine threshold for addiction—the implications for tobacco regulation. New Engl. J. Med. 331: 123-125.
Chintapakorn, Y. and Hamill, J. D. 2003. Antisense-mediated down-regulation of putrescine N-methyltransferase activity in transgenic Nicotiana tabacum L. can lead to elevated levels of anatabine at the expense of nicotine. Plant Mol. Biol. 53: 87-105.
Curtin, S. J., Voytas, D. F. and Stupar, R. M. 2012. Genome engineering of crops with designer nucleases. Plant Genome 5: 42-50.
Dalton, H., Blomstedt, C. and Neale, A. 2016. Effects of down-regulating ornithine decarboxylase upon putrescine-associated metabolism and growth in Nicotiana tabacum L. J. Exp. Bot. 67: 3367-3381.
DeBoer, K. D., Dalton, H. L., Edward, F. J. and Hamill, J. D. 2011. RNAi-mediated down-regulation of ornithine decarboxylase (ODC) leads to reduced nicotine and increased anatabine levels in transgenic Nicotiana tabacum L. Phytochem. 72: 344-355.
Dewey, R. E. and Xie, J. 2013. Molecular genetics of alkaloid biosynthesis in Nicotiana tabacum. Phytochem. 94: 10-27.
Donny, E. C., Hatsukami, D. K., Benowitz, N. L., Sved, A. F., Tidey, J. W., and Cassidy, R. N. 2014. Reduced nicotine product standards for combustible tobacco: building an empirical basis for effective regulation. Prevent. Med. 68: 17-22.
Gao, H., Smith, J., Yang, M., Jones, S., Djukanovic, V., Nicholson, M. G., West, A., Bidney, D., Falco, C. S., Jantz, D. and Lyznik, L. A. 2010. Heritable targeted mutagenesis in maize using a designed endonuclease. Plant J. 61: 176-187.
Hatsukami, D. K., Kotlyar, M., Hertsgaard, L. A., Zhang, Y., Carmella, S. G., Jensen, J. A., Allen, S. S., Shields, P. G., Murphy, S. E., Stepanov, I., and Hecht, S. S. 2010. Reduced nicotine content cigarettes: effects on toxicant exposure, dependence and cessation. Addiction 105: 343-355.
Hecht, S. S. 1998. Biochemistry, biology, and carcinogenicity of tobacco-specific N-nitrosamines. Chem. Res. Toxicol. 11: 559-603.
Hecht, S. S. 2003. Tobacco carcinogens, their biomarkers and tobacco induced cancers. Nat. Rev. Cancer 3: 733-744.
Hecht, S. S., and Hoffmann, D. 1989. The relevance of tobacco-specific nitrosamines to human cancer. Cancer Surv. 8: 273-294.
Heim, W. G., Sykes, K. A., Hildreth, S. B., Sun, J., Lu, R.-H., & Jelesko, J. G. 2007. Cloning and characterization of a Nicotiana tabacum methylputrescine oxidase transcript. Phytochem. 68: 454-463.
Horsch, R. B., Fry, J. E., Hoffmann, N. L., Eichholtz, D., Rogers, S. G. and Fraley, R. T. 1985. A simple and general method for transferring genes into plants. Science 227: 1229-1231.
Kajikawa, M., Shoji, T., Kato, A. and Hashimoto, T. 2011. Vacuole-localized berberine bridge enzyme-like proteins are required for a late step of nicotine biosynthesis in tobacco. Plant Physiol. 155: 2010-2022.
Kajikawa, M., Sierro, N., Kawaguchi, H., Bakaher, N., Ivanov, N. V., Hashimoto, T. and Shoji, T. 2017. Genomic insights into the evolution of the nicotine biosynthesis pathway in tobacco. Plant Physiol. 174: 999-1011.
Katoh, A., Shoji, T. and Hashimoto, T. 2007. Molecular Cloning of N-methylputrescine oxidase from tobacco. Plant Cell Physiol. 48: 550-554.
Lewis, R. S., Bowen, S. W., Keogh, M. R. and Dewey, R. E. 2010. Three nicotine demethylase genes mediate nornicotine biosynthesis in Nicotiana tabacum L.: functional characterization of the CYP82E10 gene. Phytochem. 71: 1988-1998.
Lewis, R. S., Lopez, H. O., Bowen, S. W., Andres, K. R., Steede, W. T. and Dewey, R. E. 2015. Transgenic and mutation-based suppression of a Berberine Bridge Enzyme-Like (BBL) gene family reduces alkaloid content in field-grown tobacco. PLoS ONE 10: e0117273.
Lewis, R. S., Drake-Stowe, K. E., Heim, C. B., Steede, T., Smith, W. and Dewey, R. E. 2020. A thorough analysis of tobacco genotypes exhibiting reduced nicotine accumulation due to induced mutations in Berberine Bridge Like (BBL) genes. Frontiers Plant Sci. 11:368.
Naconsie, M., Kato, K., Shoji, T. and Hashimoto, T. 2014. Molecular evolution of N-methylputrescine oxidase in tobacco. Plant Cell Physiol. 55: 436-444.
Puchta, H. and Fauser, F. 2014. Synthetic nucleases for genome engineering in plants: prospects for a bright future. Plant J. 78: 727-741.
Shoji, T. and Hashimoto, T. 2008. Why does anatabine, but not nicotine, accumulate in jasmonate-elicited cultured tobacco BY-2 cells? Plant Cell Physiol. 49: 1209-1216.
Siminszky B., Gavilano, L, Bowen, S. W. and Dewey, R. E. 2005. Conversion of nicotine to nornicotine in Nicotiana tabacum is mediated by CYP82E4, a cytochrome P450 monooxygenase. Proc. Natl. Acad. Sci. 102: 14919-14924.
Wang, P., Zeng, J., Liang, Z., Miao, Z., Sun, X. and Tang, K. 2009. Silencing of PMT expression caused a surge of anatabine accumulation in tobacco. Mol. Biol. Rep. 36: 2285.
World Health Organization. 2015. Advisory Note: Global Nicotine Reduction Strategy. WHO Study Group on Tobacco Product Regulation. Geneva: WHO Press.
Xie, J. H., Song, W., Maksymowicz, W., Jin, W., Cheah, K., Chen, W. X., Carnes, C., Ke, J., and Conkling, M. A. 2004. Biotechnology: a tool for reduced risk tobacco products—the nicotine experience from test tube to cigarette pack. Rev. Adv. Tob. Sci. 30: 17-37.

### SEQUENCE LISTING

(5471 bp) MPO1.1 genomic sequence:
SEQ ID NO: 1
ATGgccactactaaacagaaagtgacggcaccttctccttctccttcttcttcgactgcttcttgctgtccttccac
ttctatcctccgtcgtgaggcaacagcggccattgcagtcgtgggtgacggcctgcagaattggaccaacatcccct
ccgtcgacgagaagcagaaaaagacggcctcatcagctctagcgtcattgccaaccactgaacctctttccaccaat
acctctaccaaaggtaattaataccatgctgctacataattattccatccgttctaatttatgtgtcttcatttgag
tgggcacgaactttaacaaagcagagaatgacttttaaattttgtgctcttaaattaagaatatgtgtataatatac
caaaatgtccttgaatcttgtggcttaaatatgtaatgtgaaaagttggaattaaaaagcaattaattaacatatag
aaagagatattctttttaaacggactaaaaaggagaataatacacttaaattaaaatggaggaaataatatgtacca
accgaaaggcctatatatatctatgcttgtaaagaatttttacattatccttataagtttaattacttgtagcaagt
aatttgtctcatgaaactgtcttattttcatgtagttattgttgatgtaaaaattcttttacacattgaagttaaaa
ttatgaactaatactagtttcttaaatttaactcttttccttcaggcttcagagaataattttctcggttcatgcat
gttttttgtacgcatgtgtatatatgtagtatatagttatccttaaagaattcggtgcctctatcttttatttatta
tgtttatgataatcatagttgttacgaatgcatacaaaaaaaatattcagcttaattagaaccgcagctaaggcaat
agtacctgctttaagggtccgtgagcgattaactaaatattataagatgaaggtggatgatttatttaatttaatta
gatcttgaatcttgagatcttgatatatgtgccgtttgaatgtagtgcttaattagtttcatgtttttctttaattt
ctgatcctttgagttgtggaatgattggttgcacaagaaacctatgagttgatgtgcatagcatttctaccgtttta
aaactacggatctaggtaggatacctgaaataaataatttaagagtttaaagtgatcccaacttaaatttcgagaat
caaacttcttaatattgatcgtaaattcggacataaaattttgtatgttttcttttaaataaaatttacatatttga
atcttcatagtacaacaacaacaacaacccagtataatcccacttagtggggtctgagagggtagtgtgtacgcaga
ccttacccctaccctgggtagagagactgtttccaaatagaccctcggcatccttacctccaagaactttccacctt
gctcttggggagactcgaactcacaacctcttggttgaaagtgaagaatgcttaccatcagagcaacccctcatagt
aagtcacaataattaacaattgaaaatatttaaaaggcatatgaaaaaaacggtggtcagaaaaactcgttttactc
tcaaaatgcattaaataaattgggacagagagagtaagtaattacaagtaaattttttgacaaacattaattggtaa
tgtgataaaatggttaaaatttgcgtaaaacacttttaacattgtcattgcacacaacttaaataaattagtacttt
aatatgcttttgagctttccttaagtttaagataccctgaattgatgcgaatacatatagaagtgtatgtatgattt
cctatataccactggtcctacacttaccattgcgaagtttgtttgaagattcatattaatgtttatatatggtgcag
gtatccaaatcatgacaagggctcaaacctgccatcctttggaccctttatctgctgctgagatctcagtggctgtg
gcaactgttagagctgccggtgaaacacctgaggttcttattcccttttgtacatcacttgtgtgcggcaacaagac
cactctgtcatttacaatatatatttattttaaaaaaatttcaggtcagagatgggatgcgatttattgaggtggtt
ctggtagaaccagataaaagtgtagttgcattggcagatgcatatttcttcccaccttttcagtcatcattgatgcc
gagaaccaaaggaggatctcagattcctactaagcttcctccaaggagagctaggcttattgtttacaataagaaaa
caaatgagacaagcatttggattgttgagctaaacgaagtacatgctgctgctcgaggtggacatcacaggggaaaa
gtcatcgcatccaatgttgtccctgatgttcagccacccatagtatgcacgcacttattactcacgtttagctgctg
tgtgtttgttatgatccttatattaattttttttttaatttttattgtaggatgctcaagagtatgctgaatgtgaa
gctgtggtgaaaagttatcctccctttcgagacgcaatgaggagaaggggtattgatgacttggatcttgtgatggt
tgacccttggttcgttagaatcttcttcaccagtcttcccatttcttctctattgagaaatgccgttctgttttgac
ctgaatgcactttaatttcctacaggtgtgttggttatcatagtgaggctgatgctcctagccgcaggctcgcgaaa
ccacttgtattctgcaggacagagagtgactgcccaatggaaaatggatatgcaagaccagttgaaggaatatatgt
gcttgttgatgtacaaaacatgaagattatagaatttgaagaccgaaaacttgtaccattacctccagttgacccac
tgaggaactacactgctggtgagacaagaggaggggttgatcgaagtgatgtgaaacccctacatattattcagcct
gagggtccaagctttcgtatcagtggaaactacgtagagtggcagaaggtattctcattttctcagtcttgaattgc
tttctttgtctttatataacacattgacattgttcacttcctgcgtgacagtggaactttcggattggtttcacccc
tagagagggtttagttatacactctgtggcgtatcttgatggtagcagaggtcgtagaccaatagcacataggttga
gttttgtagagatggttgtcccctatggagatccaaatgatccacattataggaagaatgcatttgatgcaggagaa
gatggccttggaaagaatgctcattcactgaagagggtcagttactatgctatgatctaaacattgcacataggtct
gaatgctctttaactttaatcgctaacgtagaacaaaatcgttactgcagggatgtgattgtttagggtacataaag
tactttgatgcccatttcacaaactttaccggaggagttgaaacgactgaaaattgtgtatgcttgcatgaagaaga
tcacggaatgctttggaagcatcaagattggagaactggccttgctgaagttagacggtctaggcgactaacagtgt
cttttgtttgtacagtggccaattatgaatatgcattctactggcatttctaccaggtaagttaatgtgttttacat
ggaatatataaactaggtattagctctccctactagtgactggtgataatgaaatatagttacaagtttgcatttgg
aacgaagagtttgctgatcataactgtctttgattcagtcaccattttatatgcaattgcaggatggaaaaattgaa
gcggaagtcaaactcactggaattcttagtttgggagcattgcaacctggagaatatcgcaaatatggtaccacaat
tttaccaggtttgtatgcaccagttcatcaacacttctttgttgcacgaatgaatatggcagttgattgtaagccag
gagaagcacacaatcaggtaactactatgactatttatagtaaacttcagttttcataatagtaactggtatgatga
atttttggaattacacaattcctttttttccaggttgttgaagtaaatgtcaaagttgaagaacctggcaaggaaaa
tgttcataataatgcattctatgctgaagaaacattgcttaggtctgaattgcaagcaatgcgtgattgtgatccat
tctctgctcgtcattggattgtaaggatccacacccgagtaactttatttattttatctcactatgaccagtgcatg
ctacataaatttgactatgaactatctgaatttgtaggttaggaacacaagaacagtaaatagaacaggacagctaa
cagggtacaagctggtacctggtccaaactgtttgccactggctggtcctgaggcgaaatttttgagaagagctgca
tttctgaagcacaatctatgggttacacaatatgcacctggagaagattttccaggaggagagttccctaatcaaaa
tccccgtgttggcgagggattagcttcttgggtcaagcaagaccggcctctggaagaaagtgatattgttctctggt
tagttctctagacaacaatcaaaactatcttactgcaatggtagtgtttgggatattgaaaaaagttctgaccagca
ccgtttattgtaggtatatttttggaatcacacatgttcctcggttggaagactggcctgttatgccagtagaacac
attggttttgtgctacaggtacttggcaatgctatgttagcttttactgtttatttcccatcttaaacatctcagtg
atttagttgattagaactcaatactggaatagcacaaaagattaaaggagaaagaattatgcaagatattcctgtaa
gagctcttaaaagaaaaagcaatttgaaagaatgctcattcactgaatcaacaaatagtaaatattgcatgaaatga
tcttagaattacatcacttactttagccggtctatcgaaaacagcttctctgccttcccaagatagaggcgtataca
ctaacctctccaaaccccacttgtgggaattcacatgctttttttgttgttgttgttgttgttgttgttgttgttgt
tgttgttgttgtactacttgaattgcatcacttgtgccaacctgcataagttgatggtcccaatctagggaagacct
tatccaactcaaatacaatctatgcagtgatactctcattctgaatttgttcttgtctttccagtttggttaatgtt
ttttctgtttgttgttgcagccacatggatactttaactgctctccggctgttgatgtccctccgccctttgcatgc
gactcagaaagcagagacagtgatgttactgaaactagtgtagcaaagtccactgccactagcttgctggccaagct
tTGA

BOLD=Start and stop codons
BOLD ITALICS=Intron sequences
UNDERLINED=The MPO1.1 sequences used to generate the RNAi constructs MPO6 and MPO10, located in exons 6 and 10, respectively.
BOLD ITALICS UNDERLINED=The location of the 22 bp target site of the custom-designed ARCUS enzyme.

(2373 bp) MPO1.1 cDNA sequence (GenBank accession #AB289456):
SEQ ID NO: 2
ATGGCCACTACTAAACAGAAAGTGACGGCACCTTCTCCTTCTCCTTCTTCTTCGACTGCTTCTTGCTGTCCTTCCAC
TTCTATCCTCCGTCGTGAGGCAACAGCGGCCATTGCAGTCGTGGGTGACGGCCTGCAGAATTGGACCAACATCCCCT
CCGTCGACGAGAAGCAGAAAAAGACGGCCTCATCAGCTCTAGCGTCATTGCCAACCACTGAACCTCTTTCCACCAAT
ACCTCTACCAAAGGTATCCAAATCATGACAAGGGCTCAAACCTGCCATCCTTTGGACCCTTTATCTGCTGCTGAGAT
CTCAGTGGCTGTGGCAACTGTTAGAGCTGCCGGTGAAACACCTGAGGTCAGAGATGGGATGCGATTTATTGAGGTGG
TTCTGGTAGAACCAGATAAAAGTGTAGTTGCATTGGCAGATGCATATTTCTTCCCACCTTTTCAGTCATCATTGATG
CCGAGAACCAAAGGAGGATCTCAGATTCCTACTAAGCTTCCTCCAAGGAGAGCTAGGCTTATTGTTTACAATAAGAA
AACAAATGAGACAAGCATTTGGATTGTTGAGCTAAACGAAGTACATGCTGCTGCTCGAGGTGGACATCACAGGGGAA
AAGTCATCGCATCCAATGTTGTCCCTGATGTTCAGCCACCCATAGATGCTCAAGAGTATGCTGAATGTGAAGCTGTG
GTGAAAAGTTATCCTCCCTTTCGAGACGCAATGAGGAGAAGGGGTATTGATGACTTGGATCTTGTGATGGTTGACCC
TTGGTGTGTTGGTTATCATAGTGAGGCTGATGCTCCTAGCCGCAGGCTCGCGAAACCACTTGTATTCTGCAGGACAG
AGAGTGACTGCCCAATGGAAAATGGATATGCAAGACCAGTTGAAGGAATATATGTGCTTGTTGATGTACAAAACATG
AAGATTATAGAATTTGAAGACCGAAAACTTGTACCATTACCTCCAGTTGACCCACTGAGGAACTACACTGCTGGTGA
GACAAGAGGAGGGGTTGATCGAAGTGATGTGAAACCCCTACATATTATTCAGCCTGAGGGTCCAAGCTTTCGTATCA
GTGGAAACTACGTAGAGTGGCAGAAGTGGAACTTTCGGATTGGTTTCACCCCTAGAGAGGGTTTAGTTATACACTCT
GTGGCGTATCTTGATGGTAGCAGAGGTCGTAGACCAATAGCACATAGGTTGAGTTTTGTAGAGATGGTTGTCCCCTA
TGGAGATCCAAATGATCCACATTATAGGAAGAATGCATTTGATGCAGGAGAAGATGGCCTTGGAAAGAATGCTCATT
CACTGAAGAGGGGATGTGATTGTTTAGGGTACATAAAGTACTTTGATGCCCATTTCACAAACTTTACCGGAGGAGTT
GAAACGACTGAAAATTGTGTATGCTTGCATGAAGAAGATCACGGAATGCTTTGGAAGCATCAAGATTGGAGAACTGG
CCTTGCTGAAGTTAGACGGTCTAGGCGACTAACAGTGTCTTTTGTTTGTACAGTGGCCAATTATGAATATGCATTCT
ACTGGCATTTCTACCAGGATGGAAAAATTGAAGCGGAAGTCAAACTCACTGGAATTCTTAGTTTGGGAGCATTGCAA
CCTGGAGAATATCGCAAATATGGTACCACAATTTTACCAGGTTTGTATGCACCAGTTCATCAACACTTCTTTGTTGC
ACGAATGAATATGGCAGTTGATTGTAAGCCAGGAGAAGCACACAATCAGGTTGTTGAAGTAAATGTCAAAGTTGAAG
AACCTGGCAAGGAAAATGTTCATAATAATGCATTCTATGCTGAAGAAACATTGCTTAGGTCTGAATTGCAAGCAATG
CGTGATTGTGATCCATTCTCTGCTCGTCATTGGATTGTTAGGAACACAAGAACAGTAAATAGAACAGGACAGCTAAC
AGGGTACAAGCTGGTACCTGGTCCAAACTGTTTGCCACTGGCTGGTCCTGAGGCGAAATTTTTGAGAAGAGCTGCAT
TTCTGAAGCACAATCTATGGGTTACACAATATGCACCTGGAGAAGATTTTCCAGGAGGAGAGTTCCCTAATCAAAAT
CCCCGTGTTGGCGAGGGATTAGCTTCTTGGGTCAAGCAAGACCGGCCTCTGGAAGAAAGTGATATTGTTCTCTGGTA
TATTTTTGGAATCACACATGTTCCTCGGTTGGAAGACTGGCCTGTTATGCCAGTAGAACACATTGGTTTTGTGCTAC
AGCCACATGGATACTTTAACTGCTCTCCGGCTGTTGATGTCCCTCCGCCCTTTGCATGCGACTCAGAAAGCAGAGAC
AGTGATGTTACTGAAACTAGTGTAGCAAAGTCCACTGCCACTAGCTTGCTGGCCAAGCTTTGA

BOLD=Start and stop codons
BOLD ITALICS UNDERLINED=The location of the 22 bp target site of the custom-designed ARCUS enzyme.

(790 aa) MPO1.1 predicted amino acid sequence:
SEQ ID NO: 3
MATTKQKVTAPSPSPSSSTASCCPSTSILRREATAAIAVVGDGLQNWTNIPSVDEKQKKTASSALASLPTTEPLSTN
TSTKGIQIMTRAQTCHPLDPLSAAEISVAVATVRAAGETPEVRDGMRFIEVVLVEPDKSVVALADAYFFPPFQSSLM
PRTKGGSQIPTKLPPRRARLIVYNKKTNETSIWIVELNEVHAAARGGHHRGKVIASNVVPDVQPPIDAQEYAECEAV
VKSYPPFRDAMRRRGIDDLDLVMVDPWCVGYHSEADAPSRRLAKPLVFCRTESDCPMENGYARPVEGIYVLVDVQNM
KIIEFEDRKLVPLPPVDPLRNYTAGETRGGVDRSDVKPLHIIQPEGPSFRISGNYVEWQKWNFRIGFTPREGLVIHS
VAYLDGSRGRRPIAHRLSFVEMVVPYGDPNDPHYRKNAFDAGEDGLGKNAHSLKRGCDCLGYIKYFDAHFTNFTGGV
ETTENCVCLHEEDHGMLWKHQDWRTGLAEVRRSRRLTVSFVCTVANYEYAFYWHFYQDGKIEAEVKLTGILSLGALQ
PGEYRKYGTTILPGLYAPVHQHFFVARMNMAVDCKPGEAHNQVVEVNVKVEEPGKENVHNNAFYAEETLLRSELQAM
RDCDPFSARHWIVRNTRTVNRTGQLTGYKLVPGPNCLPLAGPEAKFLRRAAFLKHNLWVTQYAPGEDFPGGEFPNQN
PRVGEGLASWVKQDRPLEESDIVLWYIFGITHVPRLEDWPVMPVEHIGFVLQPHGYENCSPAVDVPPPFACDSESRD
SDVTETSVAKSTATSLLAKL
(6541 bp) MPO1.2 genomic sequence:
SEQ ID NO: 4
ATGgccactactaaacagaaagtgacggcaccttcttcttcgactgctccttgctgtccttccacttccatcctccg
tcgtgaggcgacagctgccgttgcaggcgtgggcgacggcctgcaaaattggaacaacgtcccgtccgtggatgata
agcagaaaaagacggcctcatcagctctagcgtcattggcaagcactgaacctctttcctccaatacctctaccaaa
ggtaataccatactgcaacatatatctctcgctaattattccaacagtttcaatttatgtgtcttaacttgagtagg
catgaagtttaattaacaaagtcgagaatgacttttaaattaaatcttgtggtattaaactaaagatgtgcgtaatg
taccaaaatgcccggtgaaattcaaaaatagccaaatttatagtaccaaaatgcccggtgaaattcaaaaatagcca
aatttatagtggtaattaaaaaatagctacagttttaaaaataatcaaaatttagccatttttcatgtaaaaataaa
tttgaacgaaaacactgttcaaaatccgaaaaatattccagcataatatactggagttccagtataatatactggtc
cagcataatatgctggaagttcatacacatgtgctccaatctcccgtatattatgttgaaactttttgtgtgttgga
gtttcagcataatatgctgaagttcatacacaggtgtaccaatttccagtatattatgctggaactttccgtgttac
aaagttccagcaaaatattggctatttttcaatgactttataaacgctgactatttttcaattatcagtgcgaaaac
tggctagtccgtgatattttacaaaatgctcttgaattttgtggattaaatatgtcatgtgaaaagttggagttaga
aagttattaacatagaaagagatttttttttttttttttgaaattgcctaaaaaggagaataatatacataaattga
aacggacgaaaggcgtatatatatatatatatatatatgcttgtaaagaatttttacattattcttataattttaat
tacttgtagcaagtaatttgtctcatgaagctgtcattttttcatacggttacctttcatgtaaaaattcttttata
ctgtaaatgtttagaagttaaaattataaactaataggggtactagtttttaaaatttatctcttttccttcaggct
tcagagaataatatttctcggtgcatgtcttgtacgcatgtgtatatatgtagtatagttatccttaaagaattcgg
tgcctctatctttttatttattatattatgataatcatagttgttacgaatacatacaaacaaaatcttcagtttag
aaccacagctaaggcaatagtacctgctttaagggtccgtgagcgattaactaaatataagatgaaggtggatgatt
tatttaatttaattagatcttgaatcttgtgttcttgatatatgtgccgtttgaatgtagtgcttaattagtttcat
gtttttctttaatttctgatcctttgagttgtggagtgattggttgcccaagaaacctatgagttgttgttcgtagc
atttctaccgttttaaaactaaggatatgtcacttaataaattgtttaagagtttaacggtgtagaaaaggtttact
aaggtcacttaatactccctccatcgtgatttatatgatctagtttgaattttaagagtcaaatttcttaattttga
tagtaaattcgaacataaaatctttaagttttttgaaataaaatttacgtatttggaaacttcttaaaaagaactat
aagttacaataattaacaattcaaaatatttaaaaggcatatgaaaaaattacggtcaaagaaaaactcgttttgac
tctcgaagttcgaattggatcgtataagtaattataggtatatgataattaaaaatggttaaaattcgtgaaaacac
ttttaacattgtcattgtacataacttatagtacttaaacattctcttgaactttcctttagataccctaaatagat
gtgaatacataagtgcatgtatgatttcctatatatagctggttctacacttatgatttcctatatatagctggttc
tacacttaccattgccattgcttgaagattcatagagtaatattaatggtgcaggtatacaaatcatgacaagggct
cagacttgccatcctttggaccctttatctgctgctgagatctctgtggctgtggcaactgttagagctgccggtga
aacacctgaggttctgcttcttttgtacatcacttgtgtgtagaacttctttttttccgtatcggaccaacaaagga
ctaaatgtcactagaacgtggcgacaaaaccactctgtctgtcacttacaacatatttgtggaactagttttatttg
aacaaagtgacatttttttccactaattaactcttgttttatttttcctttcaaaaaaaaaaataataataatttca
ggttagagatgggatgcgatttattgaggtggttctgttagaaccagataaaagtgttgttgcattggcagatgcat
atttcttcccaccatttcagtcatcattgatgcccagaaccaaaggaggatctctaattcctactaagcttcctcca
aggagagctaggcttattgtttacaataagaaaacaaatgagacaagcatatggattgttgagctaaatgaagtaca
tgctgctgctcgaggtggacatcacaggggaaaagtcatctcatccaatgttgtccctgatgttcagccacccatag
tatgcacttaactcagctttctttctctagaacatcagataatttccggcatcgtgaaatgttaggctctatagctg
ctgtgtgtttgttatgatgcttatattaattttgttttttctgattgtaggatgctcaagagtatgctgaatgtgaa
gctgtggtgaaaagttatcctccctttcgagacgcaatgaggagaaggggtattgatgacttggatcttgtgatggt
tgacccttggttcgttagaatcttcttcaccaatcttcccatttcttctctattgagaaatgccactctgttttgac
ctgaatgcacttttatttcctacaggtgtgttggttatcatagtgaggctgatgctcctagccgcacgctcgcgaaa
ccacttgtattctccaggacagagagtgactgcccaatggaaaatggatatgcaagaccagttgaaggaatatatgt
gcttgttgatgtacaaaacatgcagattatagaatttgaagaccgaaaacttgtaccgttacctccagctgatccac
tgaggaactacactgctggtgagacaagaggaggggttgatcgaagtgatgtgaaacccctacatattattcagccc
gagggtccaagctttcgtatcagtggaaactacatagagtggcagaaggtattctcattttttcagtcttgttttgg
tatgaagcattctttgtctttatataatacattgacattgttcacttcctgcgtgacagtggaactttcggattggt
ttcacccctagagagggtttagttatacactctgtggcgtatcttgatggtagcagaggtcgcagaccaatagcaca
taggttgagttttgtagagatggttgtcccttatggggatccaaatgatccacattataggaagaatgcatttgatg
caggagaagatggccttggaaagaatgctcattcactgaagagggtcagttactattctatgttctaaacattgcag
atctgaattcttttcactttaattgctaatgtcgaacaaaataattgctgcagggatgtgattgtttagggtacata
aagtactttgatgcccatttcacaaactttacgggaggagttgaaacgactgaaaattgtgtatgcttgcatgaaga
agatcacggaatgctttggaagcatcaagattggagaactggccttgctgaagttagacggtctaggcgactaacgg
tgtcttttgtttgtacagtggccaattatgaatatgcattctactggcacttctaccaagtaagttaatgtgtttta
catggattatataaactaggtattagctctcccaacacgactggtgataatgaaacataattacaagtttgcatttg
gaatttcttcagggaaacgaagagtttgctgatcataattgtctttgattcagttaccattttacaactgtttgtac
gctatatgcaattgcaggatggaaaaattgaagcggaagtcaaactcactggaatacttagtttgggagcattgcaa
cctggagaatatcgcaaatatggtaccacaattttaccagggttgtatgcaccagttcatcaacacttctttgttgc
gcgaatgaatatggcagttgattgtaagccaggagaagcacacaatcaggtaactactatgactatttatagtaaac
ttcagttttcataatagtaactggtatagatcaggttccttctaatctggtatgattaattttttgcgttatacaaa
tatttttttccaggttgttgaagtaaatgtcaaagttgaagaacctggcaaggaaaatgttcacaataatgcattct
atgctgaagaaacattgcttaggtctgaattgcaagcaatgcgtgactgtgatccattctctgctcgtcattggatt
gtaagaatccacacctgactgtatttacctttaattgcttttatctcactatgataactagaaaggaggatagacct
cttgtttctctgtaaagcattttatataacaagtttttttactgaactgtatgttctggaactgaaactgggcacct
ttcttatgtcgtccaatttttgactaatttgatcctaaagacccttttgatgcttggttgtggttttgatcttaaga
ccagtgcatgctacataaatttgacaatgaactatctgactttgtaggttaggaacacaagaactgtaaatagaaca
ggacagctaacagggtacaagctggtacctggtccaaactgtttgccattggctggtcctgaggcgaaatttttgag
aagagctgcatttctgaagcacaatctatgggttacacaatatgcacctggagaagaatttccaggaggagagttcc
ctaatcaaaatccccgtgttggcgagggattagcttcatgggtcaagcaagaccggcctctagaagaaagtgatatt
gttctctggttagttctctagacaacaatcaaaactatcttactgcagtggatatttgagatttaaaatagtctgca
aaatgtcaaatggtcgaaactttctagacgtggaaccttctaaagtacttgaacaaatttatatcaaattcacgcat
gactagttaaatattctgtaaacttttctttttctttctttggtggatattgaaaagtatgctttgaagttttgaca
acaccgtttattgtaggtatatttttggaatcacacatgttcctcggttggaagactggcctgttatgccagtggaa
cacattggttttgtgctacaggtagttgagaatgctatgttagcttttactgtttatttcccatcttaaacatctca
gtgatttagctgagtagaactcaatactggaaaagcatattagattaaaggtaaagaattatgcaagatattccggt
acgagatcttaaaagaaaaagcaattcgaaagaatgctcattcactgaagaggctctggggaagggattcttaatca
acaaatagtaagtattgcatgaaatgatcttagaattctccttatatatcatactctctaaaccaggttatgcgtag
cctcttagtgataaatacctccatttattcccttcacttgaattacatctcccactttatccgagggtctattggaa
acaatctctatgcctttccaagacaggggtaaggcctgcgtacacactaccttctccagaccccacttgtgggaatt
cactggatttttgttgttgttgttgttgttgtacttgaattacatcacttgcgccaacctgcataagttgatggtcc
caatctagggaagacaatccaactcaaatacaatctatgcagtgatactttaaatctgaaatttacatccttaacaa
gataagatgtaccacttagtaagaccaacttcagcatcagtagttttgctcgaaataatgaacatggtatattatac
taaatgcagcttgtgaatttgttcttgtctttccagtttgggtaatgtttttttttttttttttttttttttttttt
ctgttggtgcagccacatggattctttaactgctctccggctgttgatgtccctccgccctctgcatgcgactcgga
aagcagagacagtgatgttactgaaaccagtgtagcaaagtccactgccactagcttgctggccaagcttTGA

BOLD=Start and stop codons
BOLD ITALICS Intron sequences
BOLD ITALICS UNDERLINED=The location of the 22 bp target site of the custom-designed ARCUS enzyme.

(2361 bp) MPO1.2 cDNA sequence:
SEQ ID NO: 5
ATGgccactactaaacagaaagtgacggcaccttcttcttcgactgctccttgctgtccttccacttccatcctccg
tcgtgaggcgacagctgccgttgcaggcgtgggcgacggcctgcaaaattggaacaacgtcccgtccgtggatgata
agcagaaaaagacggcctcatcagctctagcgtcattggcaagcactgaacctctttcctccaatacctctaccaaa
ggtatacaaatcatgacaagggctcagacttgccatcctttggaccctttatctgctgctgagatctctgtggctgt
ggcaactgttagagctgccggtgaaacacctgaggttagagatgggatgcgatttattgaggtggttctgttagaac
cagataaaagtgttgttgcattggcagatgcatatttcttcccaccatttcagtcatcattgatgcccagaaccaaa
ggaggatctctaattcctactaagcttcctccaaggagagctaggcttattgtttacaataagaaaacaaatgagac
aagcatatggattgttgagctaaatgaagtacatgctgctgctcgaggtggacatcacaggggaaaagtcatctcat
ccaatgttgtccctgatgttcagccacccatagatgctcaagagtatgctgaatgtgaagctgtggtgaaaagttat
cctccctttcgagacgcaatgaggagaaggggtattgatgacttggatcttgtgatggttgacccttggtgtgttgg
ttatcatagtgaggctgatgctcctagccgcacgctcgcgaaaccacttgtattctccaggacagagagtgactgcc
caatggaaaatggatatgcaagaccagttgaaggaatatatgtgcttgttgatgtacaaaacatgcagattatagaa
tttgaagaccgaaaacttgtaccgttacctccagctgatccactgaggaactacactgctggtgagacaagaggagg
ggttgatcgaagtgatgtgaaacccctacatattattcagcccgagggtccaagctttcgtatcagtggaaactaca
tagagtggcagaagtggaactttcggattggtttcacccctagagagggtttagttatacactctgtggcgtatctt
gatggtagcagaggtcgcagaccaatagcacataggttgagttttgtagagatggttgtcccttatggggatccaaa
tgatccacattataggaagaatgcatttgatgcaggagaagatggccttggaaagaatgctcattcactgaagaggg
gatgtgattgtttagggtacataaagtactttgatgcccatttcacaaactttacgggaggagttgaaacgactgaa
aattgtgtatgcttgcatgaagaagatcacggaatgctttggaagcatcaagattggagaactggccttgctgaagt
tagacggtctaggcgactaacggtgtcttttgtttgtacagtggccaattatgaatatgcattctactggcacttct
accaagatggaaaaattgaagcggaagtcaaactcactggaatacttagtttgggagcattgcaacctggagaatat
cgcaaatatggtaccacaattttaccagggttgtatgcaccagttcatcaacacttctttgttgcgcgaatgaatat
ggcagttgattgtaagccaggagaagcacacaatcaggttgttgaagtaaatgtcaaagttgaagaacctggcaagg
aaaatgttcacaataatgcattctatgctgaagaaacattgcttaggtctgaattgcaagcaatgcgtgactgtgat
ccattctctgctcgtcattggattgttaggaacacaagaactgtaaatagaacaggacagctaacagggtacaagct
ggtacctggtccaaactgtttgccattggctggtcctgaggcgaaatttttgagaagagctgcatttctgaagcaca
atctatgggttacacaatatgcacctggagaagaatttccaggaggagagttccctaatcaaaatccccgtgttggc
gagggattagcttcatgggtcaagcaagaccggcctctagaagaaagtgatattgttctctggtatatttttggaat
cacacatgttcctcggttggaagactggcctgttatgccagtggaacacattggttttgtgctacagccacatggat
tctttaactgctctccggctgttgatgtccctccgccctctgcatgcgactcggaaagcagagacagtgatgttact
gaaaccagtgtagcaaagtccactgccactagcttgctggccaagcttTGA

BOLD=Start and stop codons
BOLD ITALICS UNDERLINED=The location of the 22 bp target site of the custom-designed ARCUS enzyme.

(786 aa) MPO1.2 Predicted amino acid sequence:
SEQ ID NO: 6
MATTKQKVTAPSSSTAPCCPSTSILRREATAAVAGVGDGLQNWNNVPSVDDKQKKTASSALASLASTEPLSSNTSTK
GIQIMTRAQTCHPLDPLSAAEISVAVATVRAAGETPEVRDGMRFIEVVLLEPDKSVVALADAYFFPPFQSSLMPRTK
GGSLIPTKLPPRRARLIVYNKKTNETSIWIVELNEVHAAARGGHHRGKVISSNVVPDVQPPIDAQEYAECEAVVKSY
PPFRDAMRRRGIDDLDLVMVDPWCVGYHSEADAPSRTLAKPLVFSRTESDCPMENGYARPVEGIYVLVDVQNMQIIE
FEDRKLVPLPPADPLRNYTAGETRGGVDRSDVKPLHIIQPEGPSFRISGNYIEWQKWNFRIGFTPREGLVIHSVAYL
DGSRGRRPIAHRLSFVEMVVPYGDPNDPHYRKNAFDAGEDGLGKNAHSLKRGCDCLGYIKYFDAHFTNFTGGVETTE
NCVCLHEEDHGMLWKHQDWRTGLAEVRRSRRLTVSFVCTVANYEYAFYWHFYQDGKIEAEVKLTGILSLGALQPGEY
RKYGTTILPGLYAPVHQHFFVARMNMAVDCKPGEAHNQVVEVNVKVEEPGKENVHNNAFYAEETLLRSELQAMRDCD
PFSARHWIVRNTRTVNRTGQLTGYKLVPGPNCLPLAGPEAKFLRRAAFLKHNLWVTQYAPGEEFPGGEFPNQNPRVG
EGLASWVKQDRPLEESDIVLWYIFGITHVPRLEDWPVMPVEHIGFVLQPHGFFNCSPAVDVPPPSACDSESRDSDVT
ETSVAKSTATSLLAKL
(7565 bp) MPO2.1 (DAO1.1) genomic sequence:
SEQ ID NO: 7
ATGgccgcaactttgcacaaggtgactcctcctcctccgactactactgcttcttgctgcccttcggcttccgcttc
tgtcatccgtcgtgagtccgccgcagcctccgtcgtggaggacgatcagcagaaacaaacgccggcgctgacgtcat
tggttaactctcaacctccttcctccaatccctccggcaaaggtataaatgcctttcgactatataaaattaagttt
ctaccctctattcaaacaatgtgtgtgtgtgtgtgtgttttagccggccgtcggacttcggagtatatgtatgtgtg
caattgcacaagttgttgtctctttctgtaatttgggcatctgcatgtttgattttcgtgtgcttaccgaatataaa
tgacagcttcgatgattttgttcatctgcactgacaaattatttctatatcctcgtaatttatatacctgtagtctt
aattttggtatacatggagaaggaaattctattgaataagctacagtagaattacggtccttaaacatttgtttagt
ctcaacaatcagggtttacttttggatgttgccagtgtagttgcacaattcctgcacttatatttttagatcaaata
atatattatttagatcactacaatatattttacccctgcaattattaacgtagagtcttgtccttaatttgaattga
cgatcaatcaaatagttaactaattcctaatttcaaacttggtgatatcaaatcatttgtatctttgtatatccatt
tcactctatactcgtgtatttttctccagaggatttatactgtaatagttaaaactatttttaggttgtatgtcaac
ctaccatgtccttccttgttttgtgaaacttaagtaaatgaaattccttttgaaaagaataagaaaagatttttttt
cctttaaaaattaagaaaagataaaaacaaagtgatacattaatggagggagcttgttattggagtagttggtaaga
tgttcagtgcttgacgattccatgtttatgcagggaaacaaatcatgccaagagctcatacatgccatcctttggac
cctttatctgctgctgaaatctctgtggctgtggcgaccgtcagagctgccggtgaaacacccgaggttcttaatca
ctgtctttttgtcatccatttttgcgttgaacaattgaacaagtattagttgaacaacaggacatcatttgctctca
attcccctaattgttttcatttatttattttttgcaaaaacattttaggtcagagatggcatgcgctttattgaggt
ggttctactggaacctgataaaagtgtcattgcactggctgatgcctatttcttcccacctttccaatcttcgttga
tgtccagaaggaaaggagggcttcccattcctactaagcttcctccaaggcgagctagacttattgtatataataag
aaaacaaatgagacaagcatatggattgttgagctagctgaagtacatgctgctgctcgaggtggacatcacaaggg
aaaagtgatttcatcctatgttgttccagatgttcagccacctatagtaagcaatttctcgtctttagaactttaag
agtttagaagtacatgaacaacagatgcaattgggttatcaaagaactgaactgagaacaccaagtaagagcaaccc
aaagttcttattacgttttaaaccaaatgctttcacacttcactctaataattgctcatgaataatgaattttaact
atattttctgaaacattaccgatttcatttattggtatactttagaacgttcacttggctagatcaaccgattaatt
aagttatgaacatttttgaagttgtgaatacctccgtcctaatggaacatttaattaattttcctagcagtaagttg
gttgttcttttgtttctatcaactggtcttgtacaactggtgctctagagattctaaacactctatgaaagaaaaga
gtcggaagaatttgcatttttcttgtacgagcatacggtcttggggcaacaaggagaaaggacacaagccaaattgg
acatccattctgattcgacaagccgattaagtgtttttcgtgctatatatacttttgcttacttatattgctctctt
cttgatgaactgtgttttctttcccatcatatggaaagtagagcatacttatacaccatagtcagaagtaatttttg
catatatagtctgtctcaactctcaagtcaacccaaaaaatgttaaagtttgcaacttttaagttttttggcttaag
tgagttgtggtataggtcagctctttaaatttcaataattctcaaattttacatatcatcttaactttgtactgatt
gtaggatgcacaagagtatgcagactgtgaagctgtagttaaaaattatcctccttttagggaagcaatgaagagaa
ggggtattgatgacatggatcttgtgatggtggacccctggttagttaaatacccttgatttttctttcttctcctt
taagaaccgatttcttatttcattgcatttcgacctgaaaatgctttccgcacaggtgcgttggttttcacagtgag
gctgatgctcctagccgcaggcttgccaaaccgctagtattctgcagatcagagagtgactgcccaatggaaaatgg
atatgcaagaccggttgaaggaatttatgtacttgttgatgtgcaaaaaatgcaggtgatagagtttgaagaccgca
aacttgtacctttacctccagctgatccactgaggaattacactgctggtgagacaagaggaggggtcgatcgaagt
gatgtgaaacccctccagattattcagccagagggtccaagctttcgagtcaatgggaactatgtggagtggcaaaa
ggtaaattcttttttccagtctgatatgcaatgaaggattatttgcttatacatctaacatgtcattctttcacttt
gttttgacagtggaacttccgagtaggtttcacccctagggagggtttggttatacactctgtggcatatattgacg
gtagtaggggtcggagacccatagcccatagattgagttttgtggagatggttgtcccctatggggatccaaatgac
ccacattacagaaagaacgcttttgatgcaggagaagatgggctcggaaagaatgctcattcacttaagagggtaac
tgacatttctatggttcaattatggatgacttttgggaatgtaagtgctaatctaacaaaatcacccttgcagggat
gcgattgtttaggatacataaagtactttgatgccaattttgcaaattttaccggaggagtagaaaccactgaaaat
tgtgtatgtttgcatgaagaagatcacgggatgctctggaagcatcaagattggagaactggccttgcagaagttag
acggtctagacgacttacagtttcttttatttgcactgtggccaattatgaatatggattctactggcacttttacc
aggcaagaataagaaaatcattatcaggaaatcctgtgtttgcatgttctaaaaaaattaggtaacaatgaagtttt
attacaagttcacattttatttagaacacttgagggaaaagaaagcaagcagaagcaagataaagaatttcttgtaa
cttcggtcgtgtttatgaataatgttttctgagagacaaactcattcctggggagaaaaaaccctcattcttggcca
aatgcttgaaacaaaaccaatgttatcattctgttataattgagttgataccaataatagctctactacttgcttat
gccttttttttgtgggcgaggggggatttgctaagccaaagacaagtgaatatcagaagtttacatctatccccgag
aaaaaaattcagaatttatgctcatttattttgtataaatgtctttactgattgtaagtacgtttagctccacttac
cattttgaaatgatctgtaaattttgtggttgcaggatgggaaaattgaagcagaaatcaaactcacaggaattctc
agtttgggagcactgcctcccggagagtctcgtaaatatggcaccacaatagcaccgggattgtatgcacctgttca
tcaacacttctttgttgctcgtatgaatatggcagttgattgtaaaccaggagaagcacacaatcaggtatttagtg
gaacaagttaccatgtccacccggaagttaaagtaatttcataccttcagtttcccagttcttaactttctaatctg
agttgttagtcctttgtctaggttgttgaagttaatgttagagttgaagaacctgggaaagaaaatgttcacaacaa
tgcattctatgctaaggaaacagtgcttacgtctgaattgcaagcaatgcgtgactgtgatactttatctgctcgtc
attggattgtaagaagccttacttgactgtatcctcctttctttttatagaactgttcttcaggttacttcctggca
cagtatctgctgagatatgtactcatagtagactgtggtccttaaacatggccactgatttttgagggagctaaatg
caatgtgtagaacctccaccttaatgctagtcttgtcatatgagaattgtttctccaaaaaaatgattttaagaact
ttctgtgacttgtatggttttggcataagagtatattatgtttcattcttctgaaaatcacgaacttttgttgagtt
ttgctctctttggctagagatgtgtttggtaacgaaaacaacaaacccagtttagtcccacaagtggggtttaggga
gggtagagtgtatgcagatcttgccccctaccttgtgaaggtagagaagttgttcccgatggacccttcgctcaagg
aaagatagaaaaagaaacaatagcatcaagcagtaacaacaacacgatgatagattaaccgaagcgaaagaaacaag
aatagggataagaataagaaatagaaacaacaggtagtaaaagaaaactaagcataaggaaatacaagagcaatact
aatactactgctatggaaaagaaatacgctcaactacctgctaaccttccaccgtaagattgttgatgaaaatttca
attgcaaattcatttagatagggcggttgaactttattgtttcagcatgtggtattgtggagtcaatttttagattc
caaaatctgctaactcaaaaaccaaggagaagtggaagaaattgaatactcgcatccccaacctagtaacagagcca
tcgatgtgactgcgaacatctgattttgcaggttaggaacacaagaacatccaatagaacaggacagctaacagggt
acaagctggtacctggccctaactgtttgccattggctggtcctaaggctaagtttttgagaagagctgcatttttg
aagcacaatctatgggttacaAaatatgcacccggagaagattttcccgggggagagttccctaatcaaaatccacg
tgttggtgagggattagcttcttgggttaagcaagatcgttctctggaagaaagtgatgttgttctctggttcgttt
ccgacaactgtcaaattatgttataacattggagttgcaggtttcagattgttttacctcacacacaaacttaactt
tcataaagagctagatgtaaaacttctaattccttggaaacccttatatacatgtttgagaagttccctattccttg
aaattctgatctacacagattattgcaggtatgtttttggaatcacacatgttcctcggttggaggactggcctgtt
atgccagttgaacatatcggttttatgcttcaggtattctggaatatggatgttttgtgtttattttgtatgtactg
gtgtttttctccttatctttgtcatcatctaacttctcgttatctattgtgttaccatttacttggtactaaagagc
tgattatgtattgtatcaattttttgcaataactcaactcattcatacattcgctatgtgtatgttacaataactag
ccaaacataaacatagagtgtagcattggaagtattggcagtggagtagaaaggatcgtgaatatccacagagaact
tgtaaaatcaaagcaattgggagctggtagtagaaatgagaaggttctttttcaagttgttatcttgacattaacct
attcaatagtccgttggatttgcttgttgctttacttttactgttccttaagctgagggtctattggaaacaacctc
tctgcctttatagggtaggggtaaggctgcgtacacactaccctccccagactgcacgtatgggattaagctgggtt
ttttgttgttgtaatagtctgttggatgttttactctgttgaacaaggaagagggtttttggggagagggagagtac
ggaagttgttaataaactgcaatggctggaaaatgtacctcagcattttttttttgtatcagtcctaataggccaag
accttaagtctggatcaggttaaccacagtaaatttagtgataaaactctccatcctttttaattactgaaatcttg
taatcttttttcgaaattgctggctaaactagagaattatatcccttcctcagaacctgtttaagtttattgcccaa
gtttagcaatgactttatccaactccaaaaacctgctgtataatattcattgtgaaaactgttaagattcatggaaa
acttaactccacgtatgtaaaattttgcatagccggtcttcaatccggataaagaagagggttgtgatatcttgatg
gctaaaccaataagttccctaactaatttggtattaacatagttgattcattgatcgatataacatggaaattcggt
tgaaaagggattagttctatactcataagtagagttggcaaaatggttaaaagaaaacagttatccacccatattat
ccatcaaaaaatgagttgaataatgaactttttaaaaacgggtcaaatatggataagacccatattatccaattaga
aaatgaataaccaacgggtttaacttttacatttataaagcctcaaattggaggttcctcaagttagaaagactagg
aattctctcaaaagtaatcatattcaagaagccatggataatatggatatttatattattcgccggttaaccagtgt
tttatccgtattaaatatgtgtcgggtcggataatttatctattttttgcattacccgttttcagctcgcccatatc
cgacccgacctgcctgccacccctactcataagtcataatagaaggtggatggttctgtttgtccataacctgttca
gtttaaagaatgattatttctgcttgttggtgcagccacatggattctttaactgctctcccgctgtagatgtacct
cctcctcggggatgtgacatggaaatcaaagacagtgatggttcagaaaatggtgtagcaaagcccactcccagtaa
tttgatggccaagcttTGA

BOLD=Start and stop codons
BOLD ITALICS Intron sequences
BOLD ITALICS UNDERLINED=The location of the 22 bp target site of the custom-designed ARCUS enzyme.
BOLD ITALICS UNDERLINED CAPITALIZED=The single polymorphism that occurs in the ARCUS® meganuclease recognition sequence.

(2325 bp) MPO2.1 (DAO1.1) cDNA sequence (GenBank accession #XM_016617592):
SEQ ID NO: 8
ATGgccgcaactttgcacaaggtgactcctcctcctccgactactactgcttcttgctgcccttcggcttccgcttc
tgtcatccgtcgtgagtccgccgcagcctccgtcgtggaggacgatcagcagaaacaaacgccggcgctgacgtcat
tggttaactctcaacctccttcctccaatccctccggcaaagggaaacaaatcatgccaagagctcatacatgccat
cctttggaccctttatctgctgctgaaatctctgtggctgtggcgaccgtcagagctgccggtgaaacacccgaggt
cagagatggcatgcgctttattgaggtggttctactggaacctgataaaagtgtcattgcactggctgatgcctatt
tcttcccacctttccaatcttcgttgatgtccagaaggaaaggagggcttcccattcctactaagcttcctccaagg
cgagctagacttattgtatataataagaaaacaaatgagacaagcatatggattgttgagctagctgaagtacatgc
tgctgctcgaggtggacatcacaagggaaaagtgatttcatcctatgttgttccagatgttcagccacctatagatg
cacaagagtatgcagactgtgaagctgtagttaaaaattatcctccttttagggaagcaatgaagagaaggggtatt
gatgacatggatcttgtgatggtggacccctggtgcgttggttttcacagtgaggctgatgctcctagccgcaggct
tgccaaaccgctagtattctgcagatcagagagtgactgcccaatggaaaatggatatgcaagaccggttgaaggaa
tttatgtacttgttgatgtgcaaaaaatgcaggtgatagagtttgaagaccgcaaacttgtacctttacctccagct
gatccactgaggaattacactgctggtgagacaagaggaggggtcgatcgaagtgatgtgaaacccctccagattat
tcagccagagggtccaagctttcgagtcaatgggaactatgtggagtggcaaaagtggaacttccgagtaggtttca
cccctagggagggtttggttatacactctgtggcatatattgacggtagtaggggtcggagacccatagcccataga
ttgagttttgtggagatggttgtcccctatggggatccaaatgacccacattacagaaagaacgcttttgatgcagg
agaagatgggctcggaaagaatgctcattcacttaagaggggatgcgattgtttaggatacataaagtactttgatg
ccaattttgcaaattttaccggaggagtagaaaccactgaaaattgtgtatgtttgcatgaagaagatcacgggatg
ctctggaagcatcaagattggagaactggccttgcagaagttagacggtctagacgacttacagtttcttttatttg
cactgtggccaattatgaatatggattctactggcacttttaccaggatgggaaaattgaagcagaaatcaaactca
caggaattctcagtttgggagcactgcctcccggagagtctcgtaaatatggcaccacaatagcaccgggattgtat
gcacctgttcatcaacacttctttgttgctcgtatgaatatggcagttgattgtaaaccaggagaagcacacaatca
ggttgttgaagttaatgttagagttgaagaacctgggaaagaaaatgttcacaacaatgcattctatgctaaggaaa
cagtgcttacgtctgaattgcaagcaatgcgtgactgtgatactttatctgctcgtcattggattgttaggaacaca
agaacatccaatagaacaggacagctaacagggtacaagctggtacctggccctaactgtttgccattggctggtcc
taaggctaagtttttgagaagagctgcatttttgaagcacaatctatgggttacaAaatatgcacccggagaagatt
ttcccgggggagagttccctaatcaaaatccacgtgttggtgagggattagcttcttgggttaagcaagatcgttct
ctggaagaaagtgatgttgttctctggtatgtttttggaatcacacatgttcctcggttggaggactggcctgttat
gccagttgaacatatcggttttatgcttcagccacatggattctttaactgctctcccgctgtagatgtacctcctc
ctcggggatgtgacatggaaatcaaagacagtgatggttcagaaaatggtgtagcaaagcccactcccagtaatttg
atggccaagcttTGA

BOLD=Start and stop codons
BOLD ITALICS UNDERLINED=The location of the 22 bp target site of the custom-designed ARCUS enzyme.
BOLD ITALICS UNDERLINED CAPITALIZED=The single polymorphism that occurs in the ARCUS® meganuclease recognition sequence.

(774 aa) MPO2.1 (DAO1.1) predicted amino acid sequence:
SEQ ID NO: 9
MAATLHKVTPPPPTTTASCCPSASASVIRRESAAASVVEDDQQKQTPALTSLVNSQPPSSNPSGKGKQIMPRAHTCH
PLDPLSAAEISVAVATVRAAGETPEVRDGMRFIEVVLLEPDKSVIALADAYFFPPFQSSLMSRRKGGLPIPTKLPPR
RARLIVYNKKTNETSIWIVELAEVHAAARGGHHKGKVISSYVVPDVQPPIDAQEYADCEAVVKNYPPFREAMKRRGI
DDMDLVMVDPWCVGFHSEADAPSRRLAKPLVFCRSESDCPMENGYARPVEGIYVLVDVQKMQVIEFEDRKLVPLPPA
DPLRNYTAGETRGGVDRSDVKPLQIIQPEGPSFRVNGNYVEWQKWNFRVGFTPREGLVIHSVAYIDGSRGRRPIAHR
LSFVEMVVPYGDPNDPHYRKNAFDAGEDGLGKNAHSLKRGCDCLGYIKYFDANFANFTGGVETTENCVCLHEEDHGM
LWKHQDWRTGLAEVRRSRRLTVSFICTVANYEYGFYWHFYQDGKIEAEIKLTGILSLGALPPGESRKYGTTIAPGLY
APVHQHFFVARMNMAVDCKPGEAHNQVVEVNVRVEEPGKENVHNNAFYAKETVLTSELQAMRDCDTLSARHWIVRNT
RTSNRTGQLTGYKLVPGPNCLPLAGPKAKFLRRAAFLKHNLWVTKYAPGEDFPGGEFPNQNPRVGEGLASWVKQDRS
LEESDVVLWYVFGITHVPRLEDWPVMPVEHIGFMLQPHGFFNCSPAVDVPPPRGCDMEIKDSDGSENGVAKPTPSNL
MAKL
(7018 bp) MPO2.2 (DAO1.2) genomic sequence:
SEQ ID NO: 10
ATGgccgcaactttgcacaaggtgactccgactactgcttcggcctccgcttctatcgcccgtcgtgagtccgccgc
agcctccgtcctggtggacgatcagcagaaacaaacgccggctctgacgtcattgcttaactctcaacctccttcct
ccaatccctctagcaaaggtatatataaatgccttttgactctatacgtaaaattaagtttctacgctctattcaaa
caatgtgtgtgtgtgtgtgttttggccggccgtcggacttcggagtatatgtatgtgtgcaattgcacaagttgttg
tctctttctgtaatttgagcatctgcatgtttgattttcgtgtgcttaccgaatataaatgacggcttcgatgattt
tcttcatctgcattcgcaaattatttctatatgctcctaatttctatacctgtagtcttaattttggtatacattcg
agaaggaaattctattgaataagctacagtagaattgcggtccttaaacatttgtttagtctcaacaatcggggttt
acttttggactttgccagtgtagttgcacaattcctgcaattatttagatcaaataatatattatttcgatcacttt
gtcctgaatttgaattgacaatcaatcaactagttaactaattcctaatttcaaattaggtgagatcaaatcatttg
tatctttgtatatccatttcgcctatgttactcggactcttcgaaaatgttgttgggtgcgtgtcggatcctacaaa
tttagtgcattttcggaggatccgacacgagtgcggcagcatttttggagagtccgagcaacatagcatttcgcttt
atactcatgtatttttctccagaggatttatactgtaatagttaaactattcttaggttgtttgtcaacctaccatg
tccttccttgtttagtgaaacttaagtagatgaagttccttttgaaaagaataagaaacgatttttttcctttaaaa
attaagaaaacataaaaacaaagtgtacatgaatggagggagcttgttattggagtagttggtaagatgttcattgt
ttgacaattccatgtttatgcagggaaacaaatcatgccaagagctcatacatgccatcctttggaccctttatctg
ctgctgaaatctctgtggctgtggcgaccgtcagagctgccggtgaaacacccgaggttcttaatcactatcttttt
gtcatccatttttgcgttgaacaatggaacaagtattagttgaacaacaggacatcatttgctctcaatttccctaa
ttgttttcatttatttatttttttgcaaacacattttaggtcagagatggcatgcgctttattgaggtggttcttct
ggaacctgataaaagtgtcgttgcactggctgatgcctatttcttcccacctttccaatcttcattgatgtccagaa
ggaaaggagggcttcccattcctactaagcttcctccaaggcgagctagacttattgcatataataagaaaacaaat
gagacaagcatatggattgttgagctagctgaagtacatgctgctgctcgaggtggacatcacaagggaaaagtgat
ttcatccaatgttgttccagatgttcagccacctatagtaagcactttctcgtctttagaactttaagagattagaa
gtacatgaacaatagatgcaattgggttatcaaagaactgaagtgagaacaccaagtaagagcaacccaaagttctt
attacatttgaaaccaaatgcattcactcttcactctaataattgctcatgaaaaatgaattttaactacattttct
gaaacattaccggtttcatttattggtatcctttagaatgttcacttggctagatgcaccgattaattaagttatga
acatttttgaagttgtgaatacctccatcctaatggaacatttagtaaattttcctagcggtaagttggttgttctt
ttgtttctatcaactggtcttgtacaactggtgctctagagattctaaacactctatgaaacaaaagagtcagaaga
atttgtatttttgttgtactagcatacggtcttggggcaacgaggagaaaggacacaagccaaattggacatccatt
ctgattccagaagccgattaagtgttttccgtgctatatatacttttgtttacttatattgctctcttcttgatgaa
ctgtgttttctttctcatcatatggaaagtagagcatatttatacaccaaaatcagaagtaatttttgcatgtatag
tctgtctcaagtcaacccaaaaaatgttaaagtttgcaacttttaagttttttggcttaagtgagttgtggtatagg
tcagctctttagatttcaataattctcaaattttacatattatcttaactctactgattgtaggatgcacaagagta
tgctgactgtgaagctgtagttaaaaattatcctccttttagggaagcaatgaagagaaggggtattgatgacatgg
atgttgtgatggtggacccctggttagttaaatacccttgatttttctttcttctcccttaagaaccgatttcttat
ttcattgcatttcgacctgaaaatgctttccgcacaggtgcgttggttatcacagtgaggctgatgctcctagccgc
aggcttgccaaaccgctagtattctgcagaacagagagtgactgcccaatggaaaatggatatgcaagaccggttga
aggaatatatgcccttgttgatgtgcaaaacatgcaggtgatagagtttgaagaccgcaaacttgtacctttacctc
cagctgatccactgaggaattacactgctggtgagacaagaggaggggtcgatcgaagtgatgtaaaacccctccag
attattcagccagagggtccaagctttcgagtcaatgggaactatgtggaatggcaaaaggtaaattcttttttcca
gtctgatatgcattgaaggattatttgcttatacatctaacatgtcattctttcactttgttttgacagtggaactt
ccgagtaggtttcacccctagggagggtttggttatacactctgtggcatatcttgacggtagcaggggtcggagac
ccatagcccataggttgagttttgtggagatggttgtcccctatggggatccaaatgacccacattacagaaagaac
gcttttgatgcaggagaagatgggctcggaaagaatgctcattcacttaagagggtaactgacatttctccggttca
actatggatgacttttgagaatgtaagtgctaatctaacaaaatcacccttgcagggatgcgattgtttaggataca
taaagtactttgatgccaattttgcaaattttactggaggagtagaaaccactgaaaattgtgtatgtttgcatgaa
gaagatcacgggatgctctggaagcatcaagattggagaactggccttgcagaagttagacggtctagacgacttac
agtttcttttatttgcactgtggccaattatgaatatggattctactggcacttataccaggcaagaataagaaaat
cattatcaggaaatcctctgtttgcatgttctaaaaaaattaggtaacattgaagtatcatcacaagttcacagttt
atttagaacacttgagggaaaagaaagcaagcagaagcaagataaagaatttcttgtaactttggtcgtgtttatga
ttaatgttttctgagagacaaactcattcctgggggagaaaaaaatccttattcttggccaaatgcttgaaacaaaa
ccaatgttatcattctgttataattgagttgataccaataatagctctactacttgcttatgccttttttttgtggg
cgaggggggatttgctaagccaaagacaagtgaatatcagaagtttacatctatccccgagaaaaaaattcagaatt
tatgctcatctattttgcataaatgtctttgctgattgtaagtacgtttagctccacttaccattttgaaatgatct
gtaaattttgtggttgcaggatgggaaaattgaagcagaagtcaaactcacaggaattctcagtttgggagcattgc
cccccggagagtctcgtaaatatggcaccacaatagcaccaggattgtatgcacctgttcatcaacacttctttgtt
gctcgtatgaatatggcagttgattgtaaaccaggagaagcacacaatcaggtatttagtggaacaagttaccgtgt
ccatccggaagttaaagtaatttcataccttcagtttcccaatttttaactttctaatctgagttgttagtcctttg
tctaggttgttgaagttaatgtaagagttgaagaacctgggaaagaaaatgttcacaacaatgcgttctatgctaag
gaaacagtgcttacgtctgaattgcaagcaatgcgggactgtgatactttatctgctcgtcattggattgtaagaag
ccttacttgactgtatcctcctttctttttacaggactgttcttcaggttacttccctagcacagtatctgctgaga
tatgtactcaatagtagactgtggtccttaaacatggccactgatttttgagggagctaagtacaatgtgtagaacc
tccaccttaatgctagtattctcatatgagaaatccttacaagtgtcattcttagcatttgaacaaaaattgatcgc
cttatttctccaaaacaatgatgttaagaactttctgtgacttgtatgtgtttggcataagagtactatatgtttca
ttcttctgaaaatcacgaacttttgttgagttttgcgctctttggttagatgtgtttggtgatcttaagattacaac
aacaacgaaaacaacaaacccagtttagtcccacaagtgaggtttggggagggtagagtgtatgcagaccttacccc
ctaccttgtgaaggtagagaagttgttcccgatagacccttcgctcaaggaaagatagaaaaagaagcaatagcatc
aagcagtaacaacaacacgataatagattaacagaagcgaagaaacaagaatagggataagaataagaaatagaaac
aacaggtagtaaaagaaaactaaacataaggaaatgcaagagcaatactaatactactgctatggaaaagaaatacg
ctcaaccatacctactaaccttctaccgtaagattgcagatgaaaatttcaattgcaaattcatttagataggatgg
ttgaactttattttgtttcagcatgtggtattgtggagtcaatttttagattccaatatctgctagctcaaaacatc
aaggagaagtggaagaaattgaatactagcatccccaaccgtagtagcagagccatcgatttgactgcgaacatctg
attttgcaggttaggaacacaagaacatccaatagaacaggacagctaacagggtacaagctggtacctggccctag
ctgtttgccattagctggtcctgaggctaagtttttgagaagagctgcatttttgaagcacaatctatgggttacac
aatatgcacccggagaagattttccagggggagagttccctaatcaaaatccacgtgttggtgagggattagcttct
tgggttaagcaagatcgttctctggaagaaagtgatgttgttctctggttagtttctcgacagctgtcaattatgtt
attacattggagttgctggtttcagattgttttgccaaacacacacactttaactttcataaagagctagatgtgga
acttctaattccttggaaacctttatatacatgtttgagaagttacctattccttgaaattctgatctacacagatt
attacaggtatgtttttggaatcacacatgttcctcggttggaggactggcctgttatgccagttgaacatatcggt
tttatgcttcaggtattctggaatatggatgttatgtgtttattttgtatgaactggtgtttttctccttatctttg
ttatcatctaacttctcgttatctattgtgttaccttttacttggtactaaagagctgattaagtattgtatcaatt
ttttgtaataactcaactcattcatacattcgctgtgtgtatgttagaataactagccaaacataaccttagagtgt
agcattgaaagtattggcagtggagtagaaaggatcgtgaatatccacagagatcttgaaaatcaaagcaattggga
gctggtagtagaaatgagaaggttctttttcaagttgttatcttgacattaagtctggatcaggttaaccacagtaa
gtttagtgataaaactctccatcctttttaattactgaaatctcgtaatcttttttcgaaattgctggataaactag
agaattatatctcttcctcagaacctgtttaagtttattgtccaagtttagcaatgactttatccaactccaaaaac
ccgctggataatattcattgtgaaaactgttaagtttccggctgcctactggttaagaagattcatggaaaacttaa
ctccacctatgtaaaattttgcatagccgatcttcaatccggataaagaagagggttgtgatatcttgacggctaaa
acaataagttccctaactaatttggtattgaaacatagttgattcattgatcgatataacatggaaattcggttgaa
aagggtttagttctatactcataagtcataatagaaggtggatggttccgtttgtccataacctcttcagtttaaag
aacgattatttctgcttgttggtgcagccgcatggattctttaactgctctcctgctgtagatgtacctcctcctcg
gggatgtgacttggaaatcaaagacagtgatggttcagaaaatggtgtagcaaagcccactcccagtagtttgatgg
ccaagcttTGA

BOLD=Start and stop codons
BOLD ITALICS Intron sequences
BOLD ITALICS UNDERLINED=The location of the 22 bp target site of the custom-designed ARCUS enzyme.

(2301 bp) MPO2.2 (DAO1.2) cDNA sequence (GenBank accession #AB289457):
SEQ ID NO: 11
ATGgccgcaactttgcacaaggtgactccgactactgcttcggcctccgcttctatcgcccgtcgtgagtccgccgc
agcctccgtcctggtggacgatcagcagaaacaaacgccggctctgacgtcattgcttaactctcaacctccttcct
ccaatccctctagcaaagggaaacaaatcatgccaagagctcatacatgccatcctttggaccctttatctgctgct
gaaatctctgtggctgtggcgaccgtcagagctgccggtgaaacacccgaggtcagagatggcatgcgctttattga
ggtggttcttctggaacctgataaaagtgtcgttgcactggctgatgcctatttcttcccacctttccaatcttcat
tgatgtccagaaggaaaggagggcttcccattcctactaagcttcctccaaggcgagctagacttattgcatataat
aagaaaacaaatgagacaagcatatggattgttgagctagctgaagtacatgctgctgctcgaggtggacatcacaa
gggaaaagtgatttcatccaatgttgttccagatgttcagccacctatagatgcacaagagtatgctgactgtgaag
ctgtagttaaaaattatcctccttttagggaagcaatgaagagaaggggtattgatgacatggatgttgtgatggtg
gacccctggtgcgttggttatcacagtgaggctgatgctcctagccgcaggcttgccaaaccgctagtattctgcag
aacagagagtgactgcccaatggaaaatggatatgcaagaccggttgaaggaatatatgcccttgttgatgtgcaaa
acatgcaggtgatagagtttgaagaccgcaaacttgtacctttacctccagctgatccactgaggaattacactgct
ggtgagacaagaggaggggtcgatcgaagtgatgtaaaacccctccagattattcagccagagggtccaagctttcg
agtcaatgggaactatgtggaatggcaaaagtggaacttccgagtaggtttcacccctagggagggtttggttatac
actctgtggcatatcttgacggtagcaggggtcggagacccatagcccataggttgagttttgtggagatggttgtc
ccctatggggatccaaatgacccacattacagaaagaacgcttttgatgcaggagaagatgggctcggaaagaatgc
tcattcacttaagaggggatgcgattgtttaggatacataaagtactttgatgccaattttgcaaattttactggag
gagtagaaaccactgaaaattgtgtatgtttgcatgaagaagatcacgggatgctctggaagcatcaagattggaga
actggccttgcagaagttagacggtctagacgacttacagtttcttttatttgcactgtggccaattatgaatatgg
attctactggcacttataccaggatgggaaaattgaagcagaagtcaaactcacaggaattctcagtttgggagcat
tgccccccggagagtctcgtaaatatggcaccacaatagcaccaggattgtatgcacctgttcatcaacacttcttt
gttgctcgtatgaatatggcagttgattgtaaaccaggagaagcacacaatcaggttgttgaagttaatgtaagagt
tgaagaacctgggaaagaaaatgttcacaacaatgcgttctatgctaaggaaacagtgcttacgtctgaattgcaag
caatgcgggactgtgatactttatctgctcgtcattggattgttaggaacacaagaacatccaatagaacaggacag
ctaacagggtacaagctggtacctggccctagctgtttgccattagctggtcctgaggctaagtttttgagaagagc
tgcatttttgaagcacaatctatgggttacacaatatgcacccggagaagattttccagggggagagttccctaatc
aaaatccacgtgttggtgagggattagcttcttgggttaagcaagatcgttctctggaagaaagtgatgttgttctc
tggtatgtttttggaatcacacatgttcctcggttggaggactggcctgttatgccagttgaacatatcggttttat
gcttcagccgcatggattctttaactgctctcctgctgtagatgtacctcctcctcggggatgtgacttggaaatca
aagacagtgatggttcagaaaatggtgtagcaaagcccactcccagtagtttgatggccaagcttTGA

BOLD=Start and stop codons
BOLD ITALICS UNDERLINED=The location of the 22 bp target site of the custom-designed ARCUS enzyme.

(766 aa) MPO2.2 (DAO1.2) predicted amino acid sequence:
SEQ ID NO: 12
MAATLHKVTPTTASASASIARRESAAASVLVDDQQKQTPALTSLLNSQPPSSNPSSKGKQIMPRAHTCHPLDPLSAA
EISVAVATVRAAGETPEVRDGMRFIEVVLLEPDKSVVALADAYFFPPFQSSLMSRRKGGLPIPTKLPPRRARLIAYN
KKTNETSIWIVELAEVHAAARGGHHKGKVISSNVVPDVQPPIDAQEYADCEAVVKNYPPFREAMKRRGIDDMDVVMV
DPWCVGYHSEADAPSRRLAKPLVFCRTESDCPMENGYARPVEGIYALVDVQNMQVIEFEDRKLVPLPPADPLRNYTA
GETRGGVDRSDVKPLQIIQPEGPSFRVNGNYVEWQKWNFRVGFTPREGLVIHSVAYLDGSRGRRPIAHRLSFVEMVV
PYGDPNDPHYRKNAFDAGEDGLGKNAHSLKRGCDCLGYIKYFDANFANFTGGVETTENCVCLHEEDHGMLWKHQDWR
TGLAEVRRSRRLTVSFICTVANYEYGFYWHLYQDGKIEAEVKLTGILSLGALPPGESRKYGTTIAPGLYAPVHQHFF
VARMNMAVDCKPGEAHNQVVEVNVRVEEPGKENVHNNAFYAKETVLTSELQAMRDCDTLSARHWIVRNTRTSNRTGQ
LTGYKLVPGPSCLPLAGPEAKFLRRAAFLKHNLWVTQYAPGEDFPGGEFPNQNPRVGEGLASWVKQDRSLEESDVVL
WYVFGITHVPRLEDWPVMPVEHIGFMLQPHGFFNCSPAVDVPPPRGCDLEIKDSDGSENGVAKPTPSSLMAKL
(155 bp) MPO6 nucleic acid sequence:
SEQ ID NO: 13
gagggtttagttatacactctgtggcgtatcttgatggtagcagaggtcgtagaccaatagcacataggttgagttt
tgtagagatggttgtcccctatggagatccaaatgatccacattataggaagaatgcatttgatgcaggagaagatg
g
(242 bp) MPO10 nucleic acid sequence:
SEQ ID NO: 14
tagaacaggacagctaacagggtacaagctggtacctggtccaaactgtttgccactggctggtcctgaggcgaaat
ttttgagaagagctgcatttctgaagcacaatctatgggttacacaatatgcacctggagaagattttccaggagga
gagttccctaatcaaaatccccgtgttggcgagggattagcttcttgggtcaagcaagaccggcctctggaagaaag
tgatattgttc
(22 bp) Nucleotide sequence of MPO1.1, MPO1.2, and MPO2.2 exon 10 target site
recognized by the engineered ARCUS ® endonuclease:
SEQ ID NO: 15
ATCTATGGGTTACACAATATGC
(559 aa) BBLa polypeptide sequence
SEQ ID NO: 16
MFPLIILISFSLASLSETATGAVINLSACLINHNVHNFSIYPTSRNYFNLLHFSLQNLRF
AAPFMPKPTFIILPSSKEELVSTIFCCRKASYEIRVRCGGHSYEGTSYVSFDASPFVIVD
LMKLDDVSVDLDSETAWAQGGATIGQIYYAIAKVSDVHAFSAGSGPTVGSGGHISGGGFG
LLSRKFGLAADNVVDALLIDADGRLLDRKAMGEDVFWAIRGGGGGNWGIVYAWKIRLLKV
PKIVTTCMIYRPGSKQYVAQILEKWQIVTPNLVDDFTLGVLLRPADLPADMKYGNTTPIE
IFPQFNALYLGPKTEVLSISNETFPELGVKNDECKEMTWVESALFFSELADVNGNSTGDI
SRLKERYMDGKGFFKGKTDYVKKPVSMDGMLTFLVELEKNPKGYLVFDPYGGAMDKISDQ
AIAFPHRKGNLFAIQYLAQWNEEDDYMSDVYMEWIRGFYNTMTPFVSSSPRGAYINYLDM
DLGVNMVDDYLLRNASSSSPSSSVDAVERARAWGEMYFLHNYDRLVKAKTQIDPLNVERH
EQSIPPMLGSTQEHKYSSE
(568 aa) BBLb polypeptide sequence
SEQ ID NO: 17
MFPLIILISFSLTSLSATATSGAGGGVANLYTCLIDHNVHNFSIYPTKNDQSSSNYENLL
DESLQNLRFAASYMPKPTVIILPNSKEELVSTILCCRQASYEIRVRCGGHSYEGTSYVSF
DGSPFVIVDLMKLDEVSVDLDSETAWAQGGATIGQIYYAIAKVSDVHAFSAGSGPTVGSG
GHISGGGFGLLSRKFGLAADNVVDALLIDADGRLLDRKAMGEDVFWAIRGGGGGNWGIIY
AWKIRLLKVPKIVTTCMIYRPGSKQYVAQLLQKWQIVTPNLVDDFTLGVLLRPADLPADM
KYGNSTPIEIFPQFNALYLGPKTEVLSISNEEFPELGVKNDECKEMTWIESALFFSELAD
INGNSSNDISRLKERYMDGKGFFKGKTDYVKKPVSMDGMLTFLVELEKNPKGYLVEDPYG
GAMDKIDDQAIAFPHRKGNLFAIQYLAQWNEEDDYKSDVYMEWIRGFYNTMTPFVSSSPR
GAYINYLDMDLGVNMDDDYLLRNASSRNSSSSVDAVERARAWGEMYFLHNYDRLVKAKTQ
IDPLNVFRHEQSIPPMLGSTQEHKYSSE
(562 aa) BBLc polypeptide sequence
SEQ ID NO: 18
MFPLIILISFSFTELSASATSGAGEGVANLSTCLINHNVHNFSMYPTSRNYENLLDESLQ
NLRFAASNMPKPTVIILPNSKEELVSTILCCRQTSYEIRVRCGGHSYEGTSSVSFDGSPF
VIIDLMKLDDVSVDLDSETAWAQGGATIGQIYYAIAKASDVHAFSAGSGPTVGSGGHISG
GGFGLLSRKFGVAADSVVDALLIDADGRLLDRKAMGEDVFWAIRGGGGGNWGIIYAWKIR
LVKVPKIVTTFKISKPGSKQYVAPLLYKWQIVAPNLADDFTLGVQMIPIDLPADMKYGNP
TPIEICPQFNGLYLGPKTEAVSILNEAFPELNVKNDDAKEMTWIESALFFSDLDNIFGNS
SDDISHLKERYLGVKICFKGKSDYVKTPFSMDGIMTALVEHEKNPNAFLVFDPYGGAMDK
ISAQAIAFPHRKGNLFAIQYYAQWNEEDDAKSNEHIEWIRGFYNKMAPFVSSSPRGAYVN
YLDMDLGMNMDDDYLLRNASSRYSSSVDAVERARAWGEKYFLNNYDRLVKAKTKIDPLNV
FRHEQSIPPTLGSTQEHNYSSE

What is claimed is:

1. A tobacco product comprising tobacco from a Nicotiana plant, wherein the plant comprises:
(A) a modification that reduces:
(i) activity of BBLa, BBLb, and BBLc, or
(ii) expression of a nucleic acid encoding BBLa, a nucleic acid encoding BBLb, and a nucleic acid encoding BBLc; and
(B) a mutation in at least one endogenous polynucleotide selected from the group consisting of:
(i) an MPO1.1 polynucleotide having about 85% to about 100% sequence identity to SEQ ID NO:1, SEQ ID NO: 2, or a polynucleotide encoding the polypeptide set forth in SEQ ID NO: 3;
(ii) an MPO1.2 polynucleotide having about 85% to about 100% sequence identity to SEQ ID NO: 4, SEQ ID NO: 5, or a polynucleotide encoding the polypeptide set forth in SEQ ID NO: 6;
(iii) an MPO2.1 polynucleotide having about 85% to about 100% sequence identity to SEQ ID NO: 7, SEQ ID NO: 8, or a polynucleotide encoding the polypeptide set forth in SEQ ID NO: 9; and
(iv) an MPO2.2 polynucleotide having about 85% to about 100% sequence identity to SEQ ID NO: 10, SEQ ID NO: 11, or a polynucleotide encoding the polypeptide set forth in SEQ ID NO: 12,
wherein the Nicotiana plant has a nicotinic alkaloid content that is reduced as compared to a plant that is not modified per (A) and does not comprise the mutations of (B).

2. The tobacco product of claim 1, wherein the Nicotiana plant comprises a mutation in at least two of polynucleotides (i)-(iv).

3. The tobacco product of claim 1, wherein the Nicotiana plant comprises a mutation in at least three of polynucleotides (i)-(iv)

4. The tobacco product of claim 1, wherein the Nicotiana plant comprises a mutation in each of polynucleotides (i)-(iv).

5. The tobacco product of any one of claims 1-4, wherein the nicotinic alkaloid is nicotine.

6. The tobacco product of claim 5, wherein the Nicotiana plant comprises a nicotine content of about 0.4 mg/g or less.

7. The tobacco product of claim 6, wherein the Nicotiana plant comprises a nicotine content of about 0.1 mg/g or less.

8. The tobacco product of any one of claims 1-7, wherein the combination of modifications of (A) and the mutation of (B) has a synergistic effect in the reduction of nicotine content in the Nicotiana plant.

9. The tobacco product of claim 8, wherein the synergistic effect comprises a reduction of nicotine content in the Nicotiana plant that is greater than that resulting from either the modifications of (A) alone or the mutation of (B) alone.

10. The tobacco product of any one of claims 1-9, wherein the Nicotiana plant comprises an anatabine content that is reduced as compared to a plant that is not modified per (A) but does comprise the mutation of (B).

11. The tobacco product of any one of claims 1-10, wherein the tobacco is selected from the group consisting of leaf tobacco, shredded tobacco, cut tobacco, ground tobacco, powder tobacco, tobacco extract, smokeless tobacco, moist or dry snuff, pipe tobacco, cigar tobacco, cigarillo tobacco, cigarette tobacco, and chewing tobacco.

12. The tobacco product of any one of claims 1-11, wherein the product is selected from the group consisting of a cigarillo, a kretek cigarette, a non-ventilated recess filter cigarette, a vented recess filter cigarette, a cigar, snuff, tobacco-containing gum, tobacco-containing lozenges, and chewing tobacco.

13. A method of producing a Nicotiana plant having reduced nicotinic alkaloid content, comprising combining in a Nicotiana plant:
(A) a modification that reduces:
(i) activity of BBLa, BBLb, and BBLc or
(ii) expression of a nucleic acid encoding BBLa, a nucleic acid encoding BBLb, and a nucleic acid encoding BBLc; and
(B) a mutation in at least one endogenous polynucleotide selected from the group consisting of:
(i) an MPO1.1 polynucleotide having about 85% to about 100% sequence identity to SEQ ID NO:1, SEQ ID NO: 2, or a polynucleotide encoding the polypeptide set forth in SEQ ID NO: 3;
(ii) an MPO1.2 polynucleotide having about 85% to about 100% sequence identity to SEQ ID NO: 4, SEQ ID NO: 5, or a polynucleotide encoding the polypeptide set forth in SEQ ID NO: 6;
(iii) an MPO2.1 polynucleotide having about 85% to about 100% sequence identity to SEQ ID NO: 7, SEQ ID NO: 8, or a polynucleotide encoding the polypeptide set forth in SEQ ID NO: 9; and
(iv) an MPO2.2 polynucleotide having about 85% to about 100% sequence identity to SEQ ID NO: 10, SEQ ID NO: 11, or a polynucleotide encoding the polypeptide set forth in SEQ ID NO: 12,
wherein the Nicotiana plant has a nicotinic alkaloid content that is reduced as compared to a plant that is not modified per (A) and does not comprise the mutations of (B).

14. The method of claim 13, wherein the Nicotiana plant comprises a mutation in at least two of polynucleotides (i)-(iv).

15. The method of claim 13, wherein the Nicotiana plant comprises a mutation in at least three of polynucleotides (i)-(iv).

16. The method of claim 13, wherein the Nicotiana plant comprises a mutation in each of polynucleotides (i)-(iv).

17. The method of any one of claims 13-16, wherein generation of the mutation comprises introducing into the plant at least one RNAi plasmid that suppresses expression of a gene product encoded by one or more of polynucleotides (i)-(iv).

18. The method of claim 17, wherein the at least one RNAi plasmid comprises at least 21 consecutive nucleotides of the nucleic acid sequence set forth in SEQ ID NO: 13 or SEQ ID NO: 14 in sense and/or antisense orientation.

19. The method of any one of claims 13-16, wherein generation of the mutation comprises introducing into the plant at least one recombinant nucleic acid encoding a nuclease targeting at least one of polynucleotides (i)-(iv).

20. The method of any one of claims 13-16, wherein generation of the mutation comprises introducing into the plant at least one recombinant nucleic acid encoding a nuclease targeting at least two of polynucleotides (i)-(iv).

21. The method of any one of claims 13-16, wherein generation of the mutation comprises introducing into the plant at least one recombinant nucleic acid encoding a nuclease targeting at least three of polynucleotides (i)-(iv).

22. The method of any one of claims 13-16, wherein generation of the mutation comprises introducing into the plant at least one recombinant nucleic acid encoding a nuclease targeting each of polynucleotides (i)-(iv).

23. The method of any one of claims 19-22, wherein the nuclease comprises a meganuclease, a zinc finger nuclease (ZFN), a transcription activator-like effector nuclease (TALEN), and/or a clustered regularly interspaced short palindromic repeat (CRISPR) associated (Cas) nuclease.

24. The method of claim 23, wherein the nuclease comprises a meganuclease.

25. The method of claim 24, wherein the meganuclease is designed to recognize a target sequence comprising a 15-40 base-pair cleavage site in at least one of polynucleotides (i)-(iv).

26. The method of claim 24, wherein the meganuclease is designed to recognize a target sequence comprising a 5′-ATCTATGGGTTACACAATATGC-3′ (SEQ ID NO: 15).

27. The method of any one of claims 13-26, wherein the mutation is a deletion or an insertion.

28. The method of any one of claims 13-27, wherein the nicotinic alkaloid is nicotine.

29. The method of claim 28, wherein the Nicotiana plant comprises a nicotine content of about 0.4 mg/g or less.

30. The method of claim 29, wherein the Nicotiana plant comprises a nicotine content of about 0.1 mg/g or less.

31. The method of any one of claims 13-30, wherein the combination of modifications per (A) and the mutation of (B) has a synergistic effect in the reduction of nicotine content in the Nicotiana plant.

32. The method of claim 31, wherein the synergistic effect comprises a reduction of nicotine content in the Nicotiana plant that is greater than that resulting from either the modifications of (A) alone or the mutation of (B) alone.

33. The method of any one of claims 13-32, wherein the Nicotiana plant comprises an anatabine content that is reduced as compared to a plant that is not modified per (A) but does comprise the mutations of (B).

34. A Nicotiana plant produced by any one of the methods of claims 13-33, wherein the plant comprises:
(A) a modification that reduces:
(i) activity of BBLa, BBLb, and BBLc or
(ii) expression of a nucleic acid encoding BBLa, a nucleic acid encoding BBLb, and a nucleic acid encoding BBLc; and
(B) a mutation in at least one endogenous polynucleotide selected from the group consisting of:
(i) an MPO1.1 polynucleotide having about 85% to about 100% sequence identity to SEQ ID NO:1, SEQ ID NO: 2, or a polynucleotide encoding the polypeptide set forth in SEQ ID NO: 3;
(ii) an MPO1.2 polynucleotide having about 85% to about 100% sequence identity to SEQ ID NO: 4, SEQ ID NO: 5, or a polynucleotide encoding the polypeptide set forth in SEQ ID NO: 6;
(iii) an MPO2.1 polynucleotide having about 85% to about 100% sequence identity to SEQ ID NO: 7, SEQ ID NO: 8, or a polynucleotide encoding the polypeptide set forth in SEQ ID NO: 9; and
(iv) an MPO2.2 polynucleotide having about 85% to about 100% sequence identity to SEQ ID NO: 10, SEQ ID NO: 11, or a polynucleotide encoding the polypeptide set forth in SEQ ID NO: 12.

35. The Nicotiana plant of claim 34, wherein the plant is characterized by decreased nicotine content and decreased anatabine content as compared to a plant that is not modified per (A) and does not comprise the mutations of (B).

36. A progeny plant or seed produced from the Nicotiana plant of claim 34 or claim 35, wherein the progeny plant or seed comprises:
(A) a modification that reduces
(i) activity of BBLa, BBLb, and BBLc or
(ii) expression of a nucleic acid encoding BBLa, a nucleic acid encoding BBLb, and a nucleic acid encoding BBLc; and
(B) a mutation in at least one endogenous polynucleotide selected from the group consisting of:
(i) an MPO1.1 polynucleotide having about 85% to about 100% sequence identity to SEQ ID NO:1, SEQ ID NO: 2, or a polynucleotide encoding the polypeptide set forth in SEQ ID NO: 3;
(ii) an MPO1.2 polynucleotide having about 85% to about 100% sequence identity to SEQ ID NO: 4, SEQ ID NO: 5, or a polynucleotide encoding the polypeptide set forth in SEQ ID NO: 6;
(iii) an MPO2.1 polynucleotide having about 85% to about 100% sequence identity to SEQ ID NO: 7, SEQ ID NO: 8, or a polynucleotide encoding the polypeptide set forth in SEQ ID NO: 9; and
(iv) an MPO2.2 polynucleotide having about 85% to about 100% sequence identity to SEQ ID NO: 10, SEQ ID NO: 11, or a polynucleotide encoding the polypeptide set forth in SEQ ID NO: 12.

37. A method of producing a Nicotiana plant cell having reduced nicotinic alkaloid content, comprising combining in a Nicotiana plant cell:
(A) a modification that reduces:
(i) activity of BBLa, BBLb, and BBLc or
(ii) expression of a nucleic acid encoding BBLa, a nucleic acid encoding BBLb, and a nucleic acid encoding BBLc; and
(B) a mutation in at least one endogenous polynucleotide selected from the group consisting of:
(i) an MPO1.1 polynucleotide having about 85% to about 100% sequence identity to SEQ ID NO:1, SEQ ID NO: 2, or a polynucleotide encoding the polypeptide set forth in SEQ ID NO: 3;
(ii) an MPO1.2 polynucleotide having about 85% to about 100% sequence identity to SEQ ID NO: 4, SEQ ID NO: 5, or a polynucleotide encoding the polypeptide set forth in SEQ ID NO: 6;
(iii) an MPO2.1 polynucleotide having about 85% to about 100% sequence identity to SEQ ID NO: 7, SEQ ID NO: 8, or a polynucleotide encoding the polypeptide set forth in SEQ ID NO: 9; and
(iv) an MPO2.2 polynucleotide having about 85% to about 100% sequence identity to SEQ ID NO: 10, SEQ ID NO: 11, or a polynucleotide encoding the polypeptide set forth in SEQ ID NO: 12,
wherein the Nicotiana plant cell has a nicotinic alkaloid content that is reduced as compared to a plant cell that is not modified per (A) and does not comprise the mutations of (B).

38. The method of claim 37, wherein the Nicotiana plant cell comprises a mutation in at least two of polynucleotides (i)-(iv).

39. The method of claim 37, wherein the Nicotiana plant cell comprises a mutation in at least three of polynucleotides (i)-(iv).

40. The method of claim 37, wherein the Nicotiana plant cell comprises a mutation in each of polynucleotides (i)-(iv).

41. The method of any one of claims 37-40, wherein generation of the mutation comprises introducing into the plant cell at least one RNAi plasmid that suppresses expression of a gene product encoded by one or more of polynucleotides (i)-(iv).

42. The method of claim 41, wherein the at least one RNAi plasmid comprises at least 21 consecutive nucleotides of the nucleic acid sequence set forth in SEQ ID NO: 13 or SEQ ID NO: 14 in sense and/or antisense orientation.

43. The method of any one of claims 37-40, wherein generation of the mutation comprises introducing into the plant cell at least one recombinant nucleic acid encoding a nuclease targeting at least one of polynucleotides (i)-(iv).

44. The method of any one of claims 37-40, wherein generation of the mutation comprises introducing into the plant cell at least one recombinant nucleic acid encoding a nuclease targeting at least two of polynucleotides (i)-(iv).

45. The method of any one of claims 37-40, wherein generation of the mutation comprises introducing into the plant cell at least one recombinant nucleic acid encoding a nuclease targeting at least three of polynucleotides (i)-(iv).

46. The method of any one of claims 37-40, wherein generation of the mutation comprises introducing into the plant cell at least one recombinant nucleic acid encoding a nuclease targeting each of polynucleotides (i)-(iv).

47. The method of any one of claims 43-46, wherein the nuclease comprises a meganuclease, a zinc finger nuclease (ZFN), a transcription activator-like effector nuclease (TALEN), and/or a clustered regularly interspaced short palindromic repeat (CRISPR) associated (Cas) nuclease.

48. The method of claim 47, wherein the nuclease comprises a meganuclease.

49. The method of claim 48, wherein the meganuclease is designed to recognize a target sequence comprising a 15-40 base-pair cleavage site in at least one of polynucleotides (i)-(iv).

50. The method of claim 48, wherein the meganuclease is designed to recognize a target sequence comprising a 5′-ATCTATGGGTTACACAATATGC-3′ (SEQ ID NO: 15).

51. The method of any one of claims 37-50, wherein the mutation is a deletion or an insertion.

52. The method of any one of claims 37-51, wherein the nicotinic alkaloid is nicotine.

53. The method of claim 52, wherein the Nicotiana plant cell comprises a nicotine content of about 0.4 mg/g or less.

54. The method of claim 53, wherein the Nicotiana plant cell comprises a nicotine content of about 0.1 mg/g or less.

55. The method of any one of claims 37-54, wherein the combination of modifications per (A) and the mutation of (B) has a synergistic effect in the reduction of nicotine content in the Nicotiana plant cell.

56. The method of claim 55, wherein the synergistic effect comprises a reduction of nicotine content in the Nicotiana plant cell that is greater than that resulting from either the modifications of (A) alone or the mutation of (B) alone.

57. The method of any one of claims 37-56, wherein the Nicotiana plant cell comprises an anatabine content that is reduced as compared to a plant cell that is not modified per (A) but does comprise the mutations of (B).

58. A Nicotiana plant comprising the Nicotiana plant cell produced by the method of any one of claims 37-57, wherein the plant comprises:
(A) a modification that reduces:
(i) activity of BBLa, BBLb, and BBLc or
(ii) expression of a nucleic acid encoding BBLa, a nucleic acid encoding BBLb, and a nucleic acid encoding BBLc; and
(B) a mutation in at least one endogenous polynucleotide selected from the group consisting of:
(i) an MPO1.1 polynucleotide having about 85% to about 100% sequence identity to SEQ ID NO:1, SEQ ID NO: 2, or a polynucleotide encoding the polypeptide set forth in SEQ ID NO: 3;
(ii) an MPO1.2 polynucleotide having about 85% to about 100% sequence identity to SEQ ID NO: 4, SEQ ID NO: 5, or a polynucleotide encoding the polypeptide set forth in SEQ ID NO: 6;
(iii) an MPO2.1 polynucleotide having about 85% to about 100% sequence identity to SEQ ID NO: 7, SEQ ID NO: 8, or a polynucleotide encoding the polypeptide set forth in SEQ ID NO: 9; and
(iv) an MPO2.2 polynucleotide having about 85% to about 100% sequence identity to SEQ ID NO: 10, SEQ ID NO: 11, or a polynucleotide encoding the polypeptide set forth in SEQ ID NO: 12.

59. The Nicotiana plant of claim 58, wherein the plant is characterized by decreased nicotine content and decreased anatabine content as compared to a plant that is not modified per (A) and does not comprise the mutations of (B).

60. A progeny plant or seed produced from the Nicotiana plant of claim 58 or claim 59, wherein the progeny plant or seed comprises:
(A) a modification that reduces
(i) activity of BBLa, BBLb, and BBLc or
(ii) expression of a nucleic acid encoding BBLa, a nucleic acid encoding BBLb, and a nucleic acid encoding BBLc; and
(B) a mutation in at least one endogenous polynucleotide selected from the group consisting of:
(i) an MPO1.1 polynucleotide having about 85% to about 100% sequence identity to SEQ ID NO:1, SEQ ID NO: 2, or a polynucleotide encoding the polypeptide set forth in SEQ ID NO: 3;
(ii) an MPO1.2 polynucleotide having about 85% to about 100% sequence identity to SEQ ID NO: 4, SEQ ID NO: 5, or a polynucleotide encoding the polypeptide set forth in SEQ ID NO: 6;
(iii) an MPO2.1 polynucleotide having about 85% to about 100% sequence identity to SEQ ID NO: 7, SEQ ID NO: 8, or a polynucleotide encoding the polypeptide set forth in SEQ ID NO: 9; and
(iv) an MPO2.2 polynucleotide having about 85% to about 100% sequence identity to SEQ ID NO: 10, SEQ ID NO: 11, or a polynucleotide encoding the polypeptide set forth in SEQ ID NO: 12.
